(19) United States
(12) Patent Application Publication
(10) Pub. No.: US 2023/0303635 A1
(43) Pub. Date: Sep. 28, 2023

(54) DIAGNOSTIC METHODS AND COMPOSITIONS

(71) Applicant: THE REGENTS OF THE UNIVERSITY OF CALIFORNIA, Oakland, CA (US)

(72) Inventor: Victor Emilio MUÑOZ VAN DEN EYNDE, Merced, CA (US)

(73) Assignee: THE REGENTS OF THE UNIVERSITY OF CALIFORNIA, Oakland, CA (US)

(21) Appl. No.: 18/020,875

(22) PCT Filed: Aug. 19, 2021
(86) PCT No.: PCT/US2021/046795

§ 371 Date: Feb. 10, 2023

Related U.S. Application Data
(60) Provisional application No. 63/120,704, filed on Dec. 2, 2020.

(51) Int. Cl. C07K 14/195; G01N 33/569; G01N 33/68

(52) U.S. Cl. CPC C07K 14/195; G01N 33/569; G01N 33/68; G01N 2333/165

(57) ABSTRACT

The present disclosure provides methods for the identification and quantitation of targets (e.g., biological targets such as cells and viruses) using molecules comprising a binding moiety that binds to the target and a fluorescent moiety whose fluorescence properties are altered when the binding moiety binds to the target. A plurality of said molecules are specifically arranged in structures that resemble the size and shape of a cell to maximize affinity and sensitivity.

### CROSS REFERENCE TO RELATED APPLICATIONS

This application claims priority to U.S. Patent Application No. 63/120,704, filed Dec. 2, 2020, titled DIAGNOSTIC METHODS AND COMPOSITIONS; and U.S. patent application Ser. No. 16/997,891, filed Aug. 19, 2020, titled DIAGNOSTIC METHODS AND COMPOSITIONS, the contents of all of which are incorporated herein by reference.

### STATEMENT REGARDING FEDERALLY SPONSORED RESEARCH OR DEVELOPMENT

This invention was made with government support under Grant No. 1547848 awarded by National Science Foundation. The government has certain rights in the invention.

### FIELD

The present invention is directed to methods and systems for the ultra-high sensitive detection and identification of targets, for example, biological targets such as viruses and cells.

### BACKGROUND

The following description includes information that may be useful in understanding the present invention. It is not an admission that any of the information provided herein is prior art or relevant to the presently claimed invention, or that any publication specifically or implicitly referenced is prior art.

Proteins are considered as ideal molecular building blocks for Synthetic Biology because they are cellular nanomachines driving most biological functions. Proteins achieve such feats by self-assembling into specific 3D structures from the chemical blueprints encoded into their amino-acid sequences (or their corresponding gene). Major advances in our understanding of how proteins fold and function have been made in the last two decades. However, transforming such knowledge into engineering strategies for designing synthetic biological components with novel functionalities is a great challenge.

The holy-grail in biosensor research is to achieve specific sensing at nanoscale (molecular) resolutions in living cells and in real time. Proteins could be ideal scaffolds because of their high specificity and tunable affinity for binding, and their built-in mechanism for transducing signals through conformational changes. However, reaching nanoscale resolution in real time requires single-molecule devices that produce analog outputs. This is potentially a crippling limitation because typical proteins behave as molecular switches with inherently binary outputs.

### SUMMARY

Natural proteins are by evolutionary design capable of binding to virtually any biological target of interest through a biomolecular recognition process that involves the structural/chemical complementarity between the protein and target. Such biomolecular recognition process is highly specific and tunable in affinity/sensitivity over a relatively broad range of target concentrations (sub-pM to mM). Some proteins are also capable of changing conformation upon binding to their target and hence operate as molecular responsive devices. These two properties make proteins outstanding elements for the specific capture and detection of biological agents. However, most natural proteins do not change conformation upon binding to their targets, one classical example are antibodies.

Therefore, previous approaches based on protein biomolecular recognition either employ indirect sandwich type of assays (e.g., enzyme linked immuno sorbent assays (ELISA)) or employ specific proteins that naturally change conformation upon binding, such as the use of the protein calmodulin for implementing the Cameleon fluorescence calcium biosensors. However, such approaches are not quick, and generate large background noise and false positives.

Engineered Responsive Proteins.

The inventors herein have identified the above-mentioned issues and have further recognized that a folding-unfolding property of proteins, wherein a protein folds and unfolds gradually (one-state downhill folding), can be utilized to develop analog single-molecule devices. In particular, a selected protein may be purposely engineered to fold downhill and configured to output corresponding fluorescent signals. These engineered proteins may be configured as ultra-high sensitive and specific sensors, and may display wide dynamic range, ultrafast response and analog readouts at the single-molecule level. Proteins that are engineered to fold downhill and generate a detectable and quantifiable output (e.g., fluorescence signal) in response to binding to a target biological agent or change in physiological conditions (e.g., pH change) are referred to as engineered responsive proteins.

An example engineered responsive protein may provide a specific recognition of a biological agent, which may be the transducer of the binding event, into a physical change (change in protein conformation) and generate a corresponding response (capture or change in signal—e.g. fluorescence). Therefore, the engineered responsive protein may provide all the essential elements for the recognition of the biological agent and the direct conversion of the recognition event into a physical response, such as the capture of the biological agent or its detection via a readout signal. For certain applications, the engineered responsive protein is self-sufficient (that is, the engineered responsive protein does not require its incorporation into an assembly), such as genetically encoded fluorescence biosensors for biomedical research or for real-time monitoring of physiological parameters in living organisms (e.g., engineered proteins for pH change detection).

In one example, a method for protein engineering comprises converting a protein that binds a biological target of interest onto a responsive system to be used for one-step capture/detection applications. The method for protein engineering may be based on engineering a stability and folding mechanism of the protein so that it becomes gradually unfolded/partially disordered in the absence of target, and folds up upon binding to the target using a fraction of the binding free energy provided by the complex. This approach may be applicable to any protein and implemented based on corresponding relationships between an amino acid sequence of the protein, and protein folding and stability.

In another example, the conformational change of responsive protein may be amplified by arranging them in tandem configurations connected by structurally rigid linkers. This tandem arrangement including linkers is key for detection applications because it permits to convert the conformational changes of responsive proteins into robust ratiometric fluorescence readouts based on Forster resonance energy transfer (FRET) between two fluorophores or two fluorescent proteins attached to the responsive element. In some examples, the output signal or the sensed read-out may be based on photoinduced electron transfer (PET) or excimer pairs. In some examples, the type of read-out used may be based on the one or more structural parameters of the responsive protein. Further, additionally, in some examples, the type of read-out maybe based on whether the output is monitored in vivo or in vitro.

In this way, any natural or designed protein may be engineered and converted into a responsive element including a ratiometric output signal (that is, the output signal intensity is proportional to the detected input such as concentration of analyte, biological agent, etc.). More information and examples of the application of this technology to de novo engineering responsive proteins for fluorescence biosensing applications are provided below.

Nanoscale Assemblies

Further, the inventors have identified that for the capture/detection of a biological agent through an engineered responsive protein, extremely high affinity between the engineered responsive protein and the biological agent is required to be effective at the loads/concentrations that these agents are typically found in biologically relevant conditions. For example, the diagnostics of COVID-19 involves the detection of SARS-CoV-2 virions in patient samples (e.g. saliva specimens) that only contain about 10³ to 10⁸ viral particles per mL, and hence require the binding event to take place with affinities in the sub-attomolar range (10⁻²¹-10⁻¹⁸ M). Such concentrations are many orders of magnitude smaller than the 10⁻¹ M affinity of the interaction between streptavidin and biotin, which is the tightest protein-ligand complex known to date. Monoclonal antibodies, such as those with widespread use in sandwich detection assays (e.g. ELISA), exhibit affinities around 10⁻¹² M, at best.

For an engineered responsive protein, the (un)folding upon ligand binding of these proteins involves paying the entropic penalty of folding the protein by the binding free energy, which reduces the overall affinity of the system by typically 2-3 orders of magnitude. An engineered responsive protein includes the three basic components required for capture/detection in one molecular element: 1) biomolecular recognition (binding to agent), 2) transducer (conformational change), and 3) response (e.g. fluorescence signal readout) but require high affinities for utilization in diagnostics or capture/detection of pathogens/biomarkers that are present at very low concentrations.

The inventors herein have further recognized the above-mentioned high affinity requirement for application of engineered responsive proteins in capture and/or detection of pathogens and/or biological agents at very low concentrations, and provide methods and systems to at least partially address some of the above-mentioned requirements.

In one example, a composition comprises a nanoscale assembly of an engineered responsive proteins configured to provide one or more of amplification of binding affinity and specificity via multivalent binding for capture and detection applications and amplification of signal to noise ratio and enhanced resolution of fluorescence signals used for detection applications. The introduction of multivalency effects into responsive proteins extends their types of applications for capture/detection of biological agents to concentrations/sensitivities that are well below the limits of what is feasible using a single responsive protein. This is so because engineered responsive proteins pay an entropic penalty for their conformational transducer mechanism that reduces the binding affinity by typically 2-3 orders of magnitude. Practically, this limits their sensitivity range to nM or higher concentrations, whereas antibodies can easily reach pM (oven sub-pM) affinities. However, the implementation of multivalent binding onto responsive proteins via engineered nanoscale assemblies enables the implementation of one-step capture/detection applications down to the sensitivity range that is currently only accessible to antibodies or nucleic acid hybridization. The greatest advantage of the responsive proteins relative to antibody and nucleic-acid based technologies is that it enables one-step, instant, direct capture/detection as opposed to the use of sandwich assays or cumbersome amplification reactions.

In one example, in order to introduce and optimize multivalency into the biomolecular recognition process of responsive proteins (that is, to engineer multivalency into a given biomolecular recognition event) a method comprises engineering the responsive protein to spontaneously form nanoscale assemblies (oligomers) of specific number of copies (valency) and symmetry in configurations that enable multi-binding to the target. For a given one-to-one binding affinity is in principle possible to tune up that of the assembly through the number of monomers and their spatial configuration in reference to that of the binding target.

While the engineered responsive proteins may be self-sufficient for many applications, the implementation of responsive proteins into nanoscale assemblies improves binding affinity, sensitivity and specificity for applications that rely on the capture/detection of biological agents present in concentrations well below the nano molar range. In one example, the nano assemblies may be generated based on a combination of a responsive element and an engineered oligomerization domain onto a fusion protein that can be easily produced by recombinant means. The oligomerization domain facilitates the spontaneous formation of nanoscale assemblies of defined stoichiometry and symmetry.

In some examples, different oligomerization domains may be used to easily change the multivalency order and hence fine tune it to the structural characteristics of each binding target (e.g. optimal binding to a coronavirus particle may be of order 3 or a multiple of 3).

In some examples, a flexible amino acid linker (having a desired sequence and a threshold length) connecting the oligomerization domain and the responsive element, as well as of the point of attachment (N-terminus, C-terminus, loop), permits further optimization/tuning of binding affinity and specificity. Further, for detection purposes, the formation of a nanoscale assembly of biosensor molecules (e.g. responsive protein coupled with a fluorescence readout) may result in a customizable signal to noise enhancements for single-particle detection applications as the total fluorescence signal of the nanosensor device is multiplied by the stoichiometry of the assembly. Examples of generating nanoscale assemblies, including both static and allosterically controllable assemblies, are described below.

Functionalization of the Responsive Protein Monomers and/or their Nanoscale Assemblies onto the Surface of Microscale Particles.

In some examples, in order to boost the affinity and specificity for biological agents of the system up to the sub-attoMolar (<10⁻¹⁸ M), even zeptoMolar (10⁻²¹ M), range that is required for the capture and detection of pathogens (viruses, bacteria, other microorganisms) at the loads that are significant for diagnostics and therapeutics of infectious diseases, the responsive protein monomers and/or their nanoscale assemblies may be attached onto a surface of microscale particles. There is currently no protein-based technology available that can reach those limits because of the inherent limitations in the affinity of protein-protein complexes. The only possible option is the use of nucleic acid hybridization (the affinity can be simply enhanced by increasing the length of the oligonucleotides used for hybridization) joined to a lengthy amplification reaction (i.e. PCR) to enhance the signal.

To overcome that barrier, multivalent adhesion may be used. Multivalent adhesion is a complex multiscale process that involves the interaction between two complementary surfaces (in shape and size) that are decorated with multiple adhesion points. Each adhesion point is formed by interactions between specific protein molecules presented on each surface (e.g. attachment protein and receptor), and typically consists of nanoscale assemblies (dimers, trimers) for multivalent binding. The presence of multiple adhesion points (e.g. the 74 spike trimers of SARS-CoV2) in complementary surfaces produces an effect equivalent to molecular Velcro that strengthens the adhesive force between the two particles to unparalleled levels even when the individual protein-protein interactions are weak. In one example, multivalent adhesion may be generated through the implementation of microscale particles of defined shape and size that are decorated with responsive protein molecules or their oligomeric assemblies at specific coating densities to mimic the molecular Velcro effect thus acting as host cell decoys for the pathogen (virion or bacterium). Multivalent adhesion may also increase the specificity of the process (another typical problem of conventional antibody-based sandwich assays) enormously because any non-specific protein-protein interaction is monovalent and hence is not cooperatively enhanced by specific adhesive forces: non-specific interactions do not experience the molecular Velcro effect.

Multivalent adhesion can be specifically engineered based on the density of protein/assembly functionalization of the particles as well as their size and geometry (e.g. 1 micron diameter beads), which results in customizable sensitivity and specificity to the concentration range of interest (or to multiple ranges on multiplex arrangements) and to the characteristics of the sample. In one example, proteins may be chemically linked to beads or fibrils of different materials.

In one example, in order to generate synthetic host cell decoys for specific pathogens, controlled decoration of microscale particles with responsive proteins and/or their nanoscale assemblies may be performed. In this way, the multivalent adhesion process may be utilized to enhance the affinity and specificity of capture/detection systems for biological agents (and particularly of pathogens). Further, the generation of synthetic host cell decoy using multivalent provides a customizable solution for each biological agent. Details on how to engineer the multivalent adhesion of host decoys are provided below.

The functionalization of microscale particles with responsive proteins and/or their nanoscale assemblies provides additional key advantages for the capture/detection technology. From the capture viewpoint, the use of microscale decoys permits to physically control the adhered pathogen particles for their elimination, neutralization, or manipulation. For example, the functionalization of beads as host decoys permits to eliminate the captured pathogen particles by simple physical procedures such as centrifugation or ultrafiltration, or the use of magnetic beads will permit to retrieve the captured particles using a magnetic field. In some examples, the functionalized microscale particles may be used combination with microfluidics. In some other examples, the functionalized microscale particles with the engineered responsive proteins may be arranged on specific surfaces (fibrils) to act as specific filters/barriers that could be implemented for PPE applications or for specific physical sterilization procedures.

The implementation of microscale responsive decoys also offers fundamental advantages for pathogen detection applications (diagnostics, food safety, environment monitoring) as instant one-step detection systems with up to one virion/bacterium limits of detection without amplification reaction. This can be achieved by decorating the microscale particles with a specific biosensor for the pathogen of interest: a responsive protein that interacts with the pathogen's attachment protein (or other characteristic protein found in its surface) and contains a fluorescence readout. The functionalization of the biosensor (as monomers or oligomers) on microscale devices permits to carry out single device fluorescence imaging schemes that reduce enormously the fluorescence background and hence proportionally increase the sensitivity in detection without the use of amplification reactions.

In such systems, detection is achieved by monitoring the reporter signal (e.g. fluorescence emission) from the free and agent-complexed biosensor proteins that coat the device (e.g. beads). The key to increase sensitivity is to perform the readout from individual microscale devices (e.g. single bead imaging) so that even a single pathogen particle captured in one device can be detected from the localized change in two-color fluorescence emission. The detection can be implemented on multiple formats customized for different applications and/or throughput levels, including: plate-reader fluorescence cell imager for microscale devices microarrayed on the well's surface, high-resolution fiber-optic bundle fluorescence imaging on custom microfluidics chips functionalized with the microscale devices or on individual microscale devices by flow cytometry (fluorescence activated cell sorting).

Additional features and advantages of the disclosure will be set forth in the description that follows, and in part, will be obvious from the description; or can be learned by practice of the principles disclosed herein. The features and advantages of the disclosure can be realized and obtained by means of the instruments and combinations particularly pointed out in the appended claims. These and other features of the disclosure will become fully apparent from the following description and appended claims, or can be learned by the practice of the principles set forth herein.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 shows a schematic illustration of an example molecular rheostat based on the coupling of a signal (e.g., proton binding) to the folding ensemble of a one-state downhill folding protein module;

FIG. 2A shows a three dimensional representation of protonation of two buried histidines during pH-induced unfolding observed in a BBL domain;

FIG. 2B shows a three dimensional representation of two BBLs domains connected in tandem by a 12 amino acid helical linker (B-hel-B) as conformational amplifier;

FIG. 2C shows a three dimensional representation of two BBLs connected in tandem by a poly-proline linker, 4 prolines: B-P4-B (left) and 10 prolines: B-P10-B;

FIG. 2D shows graphs depicting steady-state bulk response of the three BBL tandems as a function of pH. B-P4-B (left) B-P10-B (center) and B-hel-B (right);

FIG. 2E shows graphs depicting steady-state bulk fluorescence intensity measurements of B-hel-B as a function of pH (left) and ionic strength dependence (right); plot 202 is pH 4, and plot 204 is pH 7;

FIG. 2F shows SM-FRET histograms of B-hel-B showing unimodal shifting distributions at different pH values. pH 4.0-5.5 (top) and pH 6.0-8.0 (bottom) as we move left to right. The two vertical lines at 0.60 and 0.72 signal the dynamic range of the overall change in signal with pH;

FIG. 2G shows simulations from experimental data to demonstrate the number of molecules required to get a FRET readout of pH with a given signal to noise ratio. (Left) 5 molecules (0.5 ms)/1 molecule (10 ms); (middle) 10 molecules (0.5 ms)/1 molecule (20 ms); (right) 50 molecules (0.5 ms)/1 molecule (100 ms). pH 4 (top plot), pH 5 (middle plot) and pH 6 (bottom plot);

FIG. 2H shows a table including slowest non-zero eigenvalue (e), which represents the relaxation rate of B-Hel-B conformational dynamics in response to histidine (de)ionization, and the mean FRET at each pH calculated using a maximum likelihood analysis of photon trajectories and 1D diffusional free energy surface model;

FIG. 21 shows two-color real-time imaging of B-Hel-B single molecules encapsulated into immobilized liposomes at different pH values as a function of time. Green is the donor and red the acceptor emission. Each snapshot corresponds to a 50 ms time point. FRET is obtained from red divided by total. pH 4 (plot 212), pH 5 (plot 214) and pH 6 (plot 216);

FIG. 3A shows a schematic illustration of an example PET quenching signal output due to protein sensor folding in response to pH change;

FIG. 3B shows graphs depicting fluorescence intensity change of different FRET constructs as a function of pH. Normalized fluorescence emission spectra of L7H (312) and M18H-F35H (314) at acidic and basic conditions and 293 K with excitation at 480 nm. FRET efficiency of L7H and M18H-F35H at various pH (316);

FIG. 3C shows fluorescence intensity change of L7H labeled with ATTO 655 at the end of the helix. Normalized fluorescence emission spectra at various pH and 293 K with excitation at 620 nm (top). Total fluorescence intensity was calculated from the area under each curve or spectrum as a pH function (bottom);

FIG. 3D shows fluorescence intensity change of L7H labeled with ATTO 655 at the C-terminal. Normalized fluorescence emission spectra at various pH and 293 K with excitation at 620 nm (top). Total fluorescence intensity was calculated from the area under each curve or spectrum as a function of pH. The inset represents the conformational change of L7H as a function of pH using the Far-UV CD technique;

FIGS. 4A, 4B, and 4C show Ca2+ sensors based on the double EF-hand calnuc domain and its folding upon binding as transducer mechanism. (Top left) scheme of the EF-hand Ca2+ binding motif. (Top right) Ultra-sharp response in the 20-80 micromolar range of the PET sensor. (Bottom) design of the FRET-based and PET-based calnuc Ca²⁺ sensors;

FIG. 4D shows graphs depicting Calnuc-based Ca²⁺ sensor based on folding upon binding and FRET readout;

FIG. 4E shows an example GFP-RFP fusion system for in vivo biosensing;

FIG. 4F shows Fluorescence activated cell sorting analysis of mammalian cells transfected with our calnuc genetically encoded calcium sensor. The top panels show the FACS selection of fluorescent cells (with sensor expressed), and the bottom panels indicate the change in FRET signal produced before and after adding 10 mM Ca2+ to the medium (and a ionofore to allow Calcium to enter the cell);

FIG. 5 shows a three dimensional representation of ATP binding domains of DesK bound to ATP;

FIG. 6A shows three dimensional representations of different assemblies and symmetries formed by variants of the BMC domain from the bacterial microcompartment protein ccmK2;

FIG. 6B shows three dimensional representations of mutational design to eliminate the interfacial interactions that lead to higher order BMC micro-assemblies;

FIG. 6C shows Cryo-EM structure of the head to tail double hexameric assembly formed by engineered CI2. Right) thermodynamic model of assembly control;

FIG. 7 shows selective stabilization of the folding transition state of the two-state folder CI2 via protein engineering. (Bottom) wild-type CI2; (top) engineered CI2;

FIG. 8A shows a three dimensional representation of a portion of the SARS-CoV-2 spike protein and the ACE2 receptor involved in complex formation. In particular, an α-helix in the peptidase domain (PD) of the ACE2 receptor, a β-sheet in the PD of the ACE2 receptor, and the receptor binding domain (RBD) of the SARS-CoV-2 spike protein are shown;

FIG. 8B shows three dimensional representations of various receptor designs including design 1, design 2a, and design 2b;

FIG. 9 shows a schematic diagram of an example system for detection of SARS-CoV-2 using hierarchical assemblies of an engineered receptor for SARS-CoV-2 spike protein;

FIG. 10A shows an illustration of SARS-CoV-2 spike trimer inserted into nanodisks or solubilized in micelles and an outline of procedure to produce spike-trimer decorated liposomes;

FIG. 10B shows an example high-throughput fluorescent bead imaging using a multimode reader/cell imager; and

FIG. 11 shows the amino acid sequence of angiotensin converting enzyme 2 (ACE2). The al-helix portion of the peptidase domain is underlined. Boldface shows portions of the ACE2 amino acid sequence present in the binding moieties (biosensors) described herein.

### DETAILED DESCRIPTION

Practice of the present disclosure employs, unless otherwise indicated, standard methods and conventional techniques in the fields of molecular diagnostics, peptide synthesis, fluorescence, virology, pathology, cell biology, molecular biology, biochemistry, cell culture, recombinant DNA and related fields as are within the skill of the art. Such techniques are described in the literature and thereby available to those of skill in the art. See, for example, Alberts, B. et al., “Molecular Biology of the Cell,” 6^{th} edition, Garland Science, New York, NY, 2015; Watson et al., “Molecular Biology of the Gene,” 7^{th} edition, Pearson, London, 2014; Lodish et al. “Molecular Cell Biology,” 8^{th} edition, W.H. Freeman, New York, NY, 2016; Voet, D. et al. “Fundamentals of Biochemistry: Life at the Molecular Level,” 5^{th} edition, John Wiley & Sons, Hoboken, N J, 2016; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R. I., “Culture of Animal Cells: A Manual of Basic Technique,” 4^{th} edition, John Wiley & Sons, Somerset, N J, 2000; and the series “Methods in Enzymology,” Academic Press, San Diego, CA.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### Definitions

A “target” is a substance that can be identified using the methods and compositions disclosed herein. In certain embodiments, the target is a biological target such as, for example, a cell or a virus.

A “target molecule” is a molecule (e.g., a protein) on the surface of a target (e.g., a cell or virus) which is capable of being bound by a binding moiety. In certain instances, the target molecule is a viral surface protein that binds to a receptor (e.g., a protein) on the surface of a cell.

A “host cell” is a cell that can be infected by a target. Generally, a host cell is a cell that can be infected by a virus, but a host cell can also be a cell that can be infected by, for example, a mycoplasma.

A “binding moiety” or “binding domain” is a component of the compositions disclosed herein that is able to bind (e.g., covalently or noncovalently) to a molecule on the surface of a target. In certain embodiments, the binding moiety is a protein or a peptide.

A “fluorescent moiety” is a component of the compositions disclosed herein that is linked (e.g., covalently) to the binding moiety. The fluorescent moiety has the property that its emission wavelength and/or the intensity of its emission is altered by the binding of the binding moiety to the target molecule. For example, certain fluorescent moieties have the property that their relative emission intensity at two wavelengths is altered by the binding of the binding moiety to the target molecule.

A composition comprising a binding moiety covalently linked to a fluorescent moiety is denoted a “biosensor.”

In certain embodiments, a biosensor is bound to a solid support, e.g., a polystyrene bead. A composition comprising a biosensor bound to a solid support is denoted a “sensor bead” or a “cell decoy.”

“Coverage density” refers to the number of biosensors that are present on the surface of a solid support.

A “detection system” is a composition comprising a biosensor. In systems utilizing a biosensor bound to a solid support, a detection system can optionally include a surface on which the solid supports are dispersed or to which the solid supports are bound (e.g., a well of a microtiter dish).

A “derivative” or “functional equivalent” of an amino acid sequence is a sequence containing one or more alterations (e.g., deletion, insertion, substitution) in the amino acid sequence that retains the function (e.g., protein binding) of the amino acid sequence. Information relating to types of alterations that do not change the function of an amino acid sequence is presented elsewhere herein.

### Engineered Responsive Proteins

Natural domains explore a continuous range of folding barriers. In one example, size-scaling effects indicate that domains <60 residues are prone to fold downhill. Further, fast-folding kinetic experiments have identified a growing group of microsecond-folding domains that belong to the downhill scenario. High stabilization from local interactions is another main determinant of downhill folding. Moreover, recent theoretical analysis suggests that highly cooperative two-state folding is rare, being found in extracellular proteins that need very slow unfolding rates to survive degradation. All of these factors together define a continuum of folding behaviors that goes from strictly two-state down to one-state downhill folding. Intrinsically disordered proteins (IDPs) would correspond to one-state downhill folders that lack tertiary interactions to fold autonomously. Thus, a significant fraction of intracellular domains are candidates for downhill folding. Downhill folding is also likely robust towards sequence manipulation because mutations tend to decrease the folding barrier at the midpoint, and thus genetic drift and protein engineering may favor downhill over two-state.

### The Conformational Rheostat.

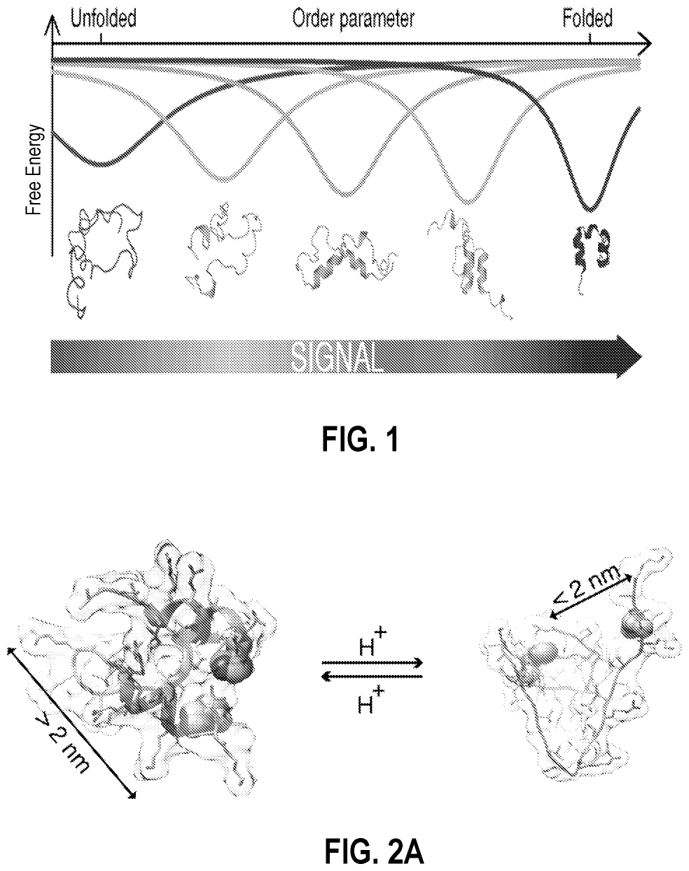

FIG. 1 shows a schematic illustration an example molecular rheostat based on the coupling of a signal (e.g., proton binding) to the folding ensemble of a one-state downhill folding protein module. Conformational rheostats are defined as devices that convert the stochastic conformational fluctuations of downhill folding protein modules onto gradual (analog) responses to specific signals. Rheostats are different from molecular motors because they only use thermal and binding energy rather than chemical energy from nucleoside triphosphate (NTP) hydrolysis. The rationale is that if the folding landscape of a protein near its denaturation midpoint (ΔG_{fold}˜0) is characterized by a free energy surface with a single global minimum (one-state downhill folding) the protein visits a continuum of partly folded conformations with micro-second Brownian dynamics. In this context rheostat effects are realized by coupling a given signal to specific conformational subsets so that binding energy gradually shifts the minimum on the folding landscape. Such gradual shift implies that the properties of individual molecules change continuously, and therefore, that their signal outputs become analog. The rheostat mechanism could also be scaled up (that is, presenting different conformational sub-ensembles for binding to multiple structurally diverse effectors) giving rise to crosstalk and other complex regulatory effects. Therefore, conformational rheostats may be used developing synthetic protein-based nanodevices. For example, protein-based nanodevices may convert the stochastic conformational fluctuations of downhill folding protein modules onto gradual (analogical) responses to specific signals.

In one example, a selected protein may be purposely engineered to fold downhill and implemented with gradual fluorescent signals. Such a protein, configured to output gradual fluorescent signal based on their folding states, may be utilized as novel sensors. The engineered responsive proteins display a wide dynamic range, provide ultrafast response, and output analog readouts at the single-molecule level.

In some examples, a downhill folding of a selected protein may be coupled with ligand binding and fluorescence readout. Further, the performance (e.g., binding affinity, dynamic range, readout signal range, and time response) of the engineered responsive protein may be adjusted as required for each particular application.

In one example, downhill folding prediction may be performed using statistical mechanical kinetic models of folding coupled to binding that are direct extensions of a free energy surface model that describes folding as a diffusive process on a 1D free energy surface (FES folding model). The FES model may be utilized for diagnosing downhill folding using standard protein folding experiments, and also for predicting downhill folding from native 3D structures. Binding is described with additional sets of coupled second-order rate equations in which each microstate on the discretized FES binds to the ligand with given on- and off-rates. The FES folding-binding models can be implemented in both kinetic (rate-matrix) and stochastic kinetic versions for the quantitative analysis of folding behavior and biosensor response in bulk and at the single-molecule level. For detailed characterization of the fluorescence readout response of target biosensors at the single-molecule level the stochastic FES folding-binding model may be combined with a recently developed maximum likelihood analysis of photon arrival times.

### Protein Engineering

In some examples, for the design of engineered mutations targeted at inducing or suppressing downhill folding behavior, a protein simulation code, such as SiMoNa/POEM++ may be utilized. This code includes ab-initio protein structure prediction and de novo protein design by stochastic optimization methods. In one example, an FES model may be parameterized empirically to predict folding and unfolding rates (and thus stability and folding barrier) based on 3D structure and sequence using a residue-residue mean-force potential refined against an experimental database of >65 single-domain proteins and >800 mutants on 24 proteins.

In one example, determination of stabilizing mutations may be performed via SiMoNa/POEM++ on the target structure restricting mutations to specific sites. Each attempted sequence may be scored according to the standard POEM++ energy function together with the FES model. Further, in some examples, cross-validation may be performed using one or more design algorithms, such as Rosetta.

In another example, the procedure to turn two-state folders onto downhill modules or vice-versa may include two steps: 1) sequence design runs on the target native 3D structure of the protein of interest; and 2) structure prediction runs using selected sequences from step 1 and POEM++(without FES) as scoring function to confirm the target structure corresponds to the global minimum. All mutational trials and designs may be first tested on molecular dynamics (MD) simulations.

### Biophysical Characterization of Downhill Folding.

One or more diagnostic tests for the identification of downhill folding may be performed using equilibrium and kinetic experiments. In various examples, diagnostic tests may be performed on new downhill folding candidates, designed mutations for increasing-decreasing downhill folding behavior, and/or proteins implemented with ratiometric fluorescence readouts. In one example, equilibrium thermodynamic tests may be performed by thermal and chemical denaturation monitored by far-UV circular dichroism (CD) (e.g. double perturbation analysis) or by a streamlined multiprobe scheme combining CD and intrinsic Tryptophan fluorescence.

Downhill folding modules are also characterized by their very fast folding rates, and thus it is possible to track the engineered conversion from two-state to downhill folder (or in reverse) by the acceleration (deceleration) of the folding rate, which needs to reach values above 20,000 s⁻¹ at room temperature to approach the downhill folding limit.

In some examples, chemical denaturation kinetic tests may be carried out with a combination of mixing methods that include conventional stopped-flow for slow and moderately fast folding times (rates below 1000 s⁻¹) and an example microfluidics continuous flow apparatus (dead time below 40 μs)

### Nuclear Magnetic Resonance (NMR).

In some examples, high-resolution multidimensional NMR experiments on uniformly isotopically (¹⁵N and ¹³C) labeled samples may be performed for the structural characterization of identified, designed and engineered downhill folding modules. The structural features of the native state for designed protein variants may be quickly tested by obtaining the full chemical shift assignment using standard procedures and then calculating a model of the structure from chemical shift data only using the program CS-Rosetta. Proteins for which there is no prior model of the native structure (downhill folding candidates with no structure available in the protein databank), a native structure may be determined using chemical shifts and Nuclear Overhauser Effects (NOEs) as structural restraints and standard computational procedures for structure calculation.

### Single-Molecule Fluorescence Spectroscopy and Microscopy.

In some examples, to characterize the fluorescence readout signals and determine their effective coupling to the relevant levels of analyte (e.g. pH, ionic strength) bulk steady-state fluorescence may be employed. For example, for characterizing the downhill-based nanosensors at the single-molecule level confocal fluorescence microscopy (SM-CFM) and total internal reflection fluorescence microscopy (SM-TIRFM) may be employed. SM-CFM may be used for all the in vitro experiments that require fast time-resolution (microseconds) and single-photon counting capabilities (photon arrival times), as well as for the fluorescence imaging of the nanosensors in living cells (in vivo imaging). For characterization of the distributions of behaviors from individual nanosensor molecules the wide-field SM-TIRFM approach may be used. These methods may be used to resolving the fast changes in fluorescence signals associated to the microsecond conformational changes of downhill folding molecules at the single-molecule level (which is essential to demonstrate their analog responses). In one example, a photoprotection method that affords photon detection fluxes of ˜1 photon per Δs combined with sophisticated maximum likelihood analysis of photon arrival times, which together afford time resolutions of 10 μs on single-molecule fluorescence experiments on proteins may be used. Further, perturbation-free labeling sites on downhill folding modules may be determined. For attaining surface immobilization, in one example, a His-tag bound to Cu² conjugated to iminodiacetic acid-polyethylenglycol derivatized glass surfaces may be used. In another example, an N-terminal insertion of an AviTag and immobilization by biotin binding to a streptavidin derivatized PEG-coated glass surface.

### Computer Simulations.

Molecular Dynamics (MD) simulations may be used to characterize the motions and structural ensembles of downhill folders (which behave like highly structured IDPs) to gain insights for the design of fluorescence readouts. MD simulations may be used also for quick testing the conformational behavior of designed mutations. Fast conformational processes will be probed with multiple ˜100s ns trajectories performed in-house employing conformational sampling methods to obtain equilibrium distributions (i.e. Replica Exchange MD simulations). All simulations may be performed with the Gromacs software package, which incorporates several force-fields, easy force-field modification, and several water models.

### Live Fluorescence Imaging in Cardiac Muscle Tissue and Heart.

To characterize an example pH-ionic strength nanosensor in living tissue and organ, a series of imaging techniques may be employed to record fluorescence and electric signals simultaneously in living tissue/organ. In one example, a Loose Patch Photolysis (LFP), a technique that allows for time measurements of membrane electric currents during a physiological action potential in the intact heart, may be employed. In another example, Fluorescence Local Field Optical Mapping Microscopy (FLOM) to obtain images of a contacting epicardial region may be used. FLOM is an epifluorescence arrangement where a high-resolution optical conduit transmits the image of the contacting epicardial region by performing recordings in more than 50,000 fibers with a 12 μm space resolution. The FLOM probe is positioned on the epicardium using a micromanipulator.

### Analog Nanosensing

In order to provide analog nanosensing, a protein may include a downhill module that exhibits effective coupling between the gradual loss (or recovery) of its structure and proportionate changes in ligand affinity, and may further include, signal readouts that track the continuous structural change. Further, the downhill-based sensing extends the dynamic range for ligand detection of both single binding sites and multiple binding sites of different affinity. The response time of these sensors (that is the proteins) is ultra-short because their microsecond conformational dynamics quickly averages out in response to the ligand, providing millisecond analog readouts.

In one example, a bi-functional pH-Ionic strength sensor based on the downhill folding domain BBL is provided. BBL is a downhill protein that unfolds at acidic pH and refolds upon increasing ionic strength. The unfolding of BBL at mildly acidic pH is due to two buried histidines (H13 and H37) that are involved in tertiary interactions (pKₐ values of ˜6.6 and ˜5.6, respectively). The conformational response of BBL and BBL+(an engineered BBL version with all the negative charges removed) to pH and ionic strength shows that BBL responds linearly over 4 orders of magnitude in [H⁺] (and similar range in ionic strength) whereas BBL+ only responds to ionic strength, thus making the combination of the two domains a bi-functional sensor. Moreover, the response to pH and ionic strength takes only microseconds and is nearly independent on ligand concentration, permitting real time monitoring of large changes in pH (rightmost panel in figure).

In order to provide analog operation at the single molecule level in vitro and also in living cells, and to implement high sensitivity fluorescence readouts that track the gradual changes in BBL structure (e.g. α-helix folding/unfolding), a Forster resonance energy transfer (FRET) readout may be used.

### pH Sensing Based on BBL:

In the BBL native 3D structure, the protein ends are ˜2.5 nm apart pointing in opposite directions, resulting in ˜100% energy transfer for relevant FRET pairs. Upon pH denaturation BBL remains compact, but its loose ends swivel resulting in even shorter distances, thus making a conventional FRET approach unviable. In one example, a head to tail BBL tandem with repeats connected by a rigid rod (protein segment designed to form a stable α-helix structure) may be constructed. Further, a rod length may be optimized to attain ˜10% FRET efficiency in the native state (high pH) whereas at lower pH, end-to-end swiveling is expected to cause a large, yet gradual FRET increase.

Ionic Strength Sensing on BBL+:

The FRET signal between the ends of intrinsically unfolded BBL+ is moderate (˜50%) and increases gradually as the protein becomes “structured” by increasing ionic strength. Further, the readout signal may be improved by grafting rigid poly-proline segments on the ends with lengths designed based on MD simulations of BBL and BBL+. Finally, a complete bi-functional pH ionic strength sensor may be built fusing the BBL-tandem and BBL+ into a single protein and using a three color FRET scheme (e.g., donor inserted between tandem-BBL and BBL+ and acceptors at both ends).

In one example, an intrinsic pH-induced conformational changes of the downhill folding protein BBL may be utilized into a high-sensitivity ratiometric fluorescence readout. As discussed above, using a downhill folding protein induces a gradual change in fluorescent signal as a function of the ligand concentration (here, protons) on an individual sensor molecule, which would enable instant analog reading of a single-molecule device through fluorescence. In addition, the use of a fast folding reaction coupled to proton binding could also lead to an ultrafast (sub-millisecond) response time.

### Engineering Downhill Folding Modules

### Building a Catalogue of Molecular Scaffolds

In one example, a set of physical-structural parameters that determine a height of the folding barrier for any given domain may be determined. These parameters can be summarized as: 1) domain size (the smaller the more downhill); 2) folding topology (antiparallel arrangements diminish the mostly-entropic folding barrier); 3) stabilization from local interactions (α-helices favor downhill folding whereas P-sheets favor two-state); 4) aliphatic-aromatic content of the hydrophobic core (fewer aromatics favor looser packing and thus downhill folding). The effects are all quantitative and thus the final folding barrier may be determined by tradeoffs between the four factors.

In one example, a physics-based algorithm may be employed for the prediction of domain downhillness from its amino acid sequence and native structure. In some examples, the native structural input may not be utilized, and a downhillness of a domain may be predicted based on its amino acid sequence. In one example, the algorithm may be based on an FES folding model, which already accounts for size effects. Contributions from factors 2-4 may be implemented by developing a knowledge-based force-field refined against kinetic and mutational experimental data. Using this algorithm, the protein data base (PDB) may be scanned for downhill folding candidates that bind to interesting targets to build a catalogue of molecular scaffolds for rheostatic sensors.

### Manipulating Folding Scenarios: From Two-State to Downhill Folding

In one example, paradigmatic two-state folding domains may be transformed into bona-fide downhill folders. In order to do so, a minimal set of sequence changes that induces such transformation may be determined.

As one example, a 64-residue CI2 domain, an extreme case of two-state folding for which stability and folding barrier seem to have been evolutionary maximized may be selected. Transformation into a downhill folding module involves minimizing the overall folding barrier and the domain stability to guarantee that the native state is conformationally flexible. This entails accelerating both folding and unfolding rates and the latter to a larger extent (to reduce stability). In this example, initially, a set of 10 mutations aiming at maximizing native secondary structure propensities, may be designed such that the core aliphatic/aromatic ratio is increased and tight packing without changing overall hydrophobicity (i.e. residue swapping) is loosened, whereas pI and accessible surface area may be kept constant. The 10-residue mutant kept the same native structure as determined by chemical shift data and CS-Rosetta and increased the minimal relaxation rate by 100-fold. However, the entire effect came from speeding the unfolding rate (rate shown in base10 log units in figure). On a second step a buried Arginine may be removed and one of the 10 mutations may be reversed to increase stability, obtaining a CI2 variant with a 3,000 fold speedup at the bottom of the chevron (minimal rate of ˜100 s⁻¹). This variant is only a factor of 200 away from the rate expected for a downhill folder at the chemical denaturation midpoint.

The CI2 folding barrier may be further decreased with single point mutations, which we will pursue one mutation at a time to avoid excessive destabilization. In addition, we will engineer the folding topology of CI2, which may be a critical factor for determining the folding barrier. The CI2 folding topology is mixed parallel, but it may be convert it into fully antiparallel by introducing a simple circular permutation that merges the two termini (strands 1 and 4) and cuts after the loop. The fully antiparallel topology should induce a significant increase in downhillness while keeping the same native structure. Next, the circular permutation may be performed on the wild-type to determine rate and stability effects, and in the fastest variants. In some examples, the conversion to downhill folding of another paradigmatic two-state domain, the cold shock protein B from T. maritima may be performed. The conversion of this domain presents different challenges and will provide complementary information because it folds moderately fast (˜3 s⁻¹ at the bottom of the chevron) and has a full antiparallel topology, but it presents an all-beta topology (least prone to downhill folding).

### Analog Single-Molecule Sensing and Fluorescence Readouts

The capabilities of downhill modules as broad-range single-molecule sensors depend on: 1) their ability to exhibit continuous binding across their conformational spectrum, 2) the capacity to link those conformational changes to high-sensitivity readouts that also respond continuously.

### Binding Scenarios for Analog Sensing

Small Ligands that Bind to Structurally Simple Sites on the Protein:

In one example, the binding site buried in the native structure (coupling binding to unfolding) may be grafted. The ligand will induce progressive domain unfolding, which in turn will proportionally increase the binding site's accessibility (and thus the effective affinity). In this scenario, dynamic range could be further increased implementing multiple sites of varying solvent accessibility.

In one example, proton binding to buried His residues and the downhill folder gpW, which does not unfold at acidic pH, thus engineering buried His residues within the gpW hydrophobic core may be utilized. The non-protonated His ring may interact favorably with hydrophobic residues, but its protonation may destabilize the native structure thus inducing unfolding. Further, multiple protonation sites may be engineered with varying solvent exposure to maximize the sensor dynamic range. As signal readouts extrinsic fluorescent probes (see below) may be introduced.

High Affinity Ligands that Bind to Structurally Complex Sites on the Protein:

In one example, the gradual entropic costs of folding a destabilized downhill module (IDP-like) may be harmonized with binding free energy provided by binding to a partly disorganized site. That is, the native ensemble presents the complete high-affinity binding site, but binding is penalized by the cost of folding the unstructured domain (which is controllable by mutation). Partially folded conformations may present incomplete binding sites (lower affinity), but also pay lower penalties to fold. The key is to balance these parameters to achieve gradual binding affinity coupled to progressive folding.

In one example, gpW may be used for binding to bi-arsenate fluorescence (FlAsH) derivatives as ligand and readout. FlAsH bind to four Cys residues increasing their quantum yield by 50,000. In short peptides bearing the sequence CCXXCC (SEQ ID NO:1), FlAsH bind with pM affinities, being highest for sequences with βturn (e.g. PG as XX) or a helical forming sequences. The 4 Cys may be incorporated to gpW in different configurations: 1) all-local (introducing the CC-XXCC sequence on one of the two gpW helices or the βturn; 2) semilocal (two CC or CXXXC (SEQ ID NO:2) stretches on two protein segments that are in close contact in the native 3D structure) since FlAsH bind to just two Cys in either i,i+1 or i,i+4 positions, but with lower affinity; and 3) global (Cys residues distributed 2-1-1 or 1-1-1-1 between the two helices and the hairpin strands of gpW, but always in spatial contact in the native structure).

Binding to Multiple Targets with Differential Affinities:

This approach may be used for multi-functional single-molecule biosensors.

In one example, the DNA-binding engrailed homeodomain (engHD), which is a 3-helix bundle downhill folding module that binds the DNA sequence TAATTA (SEQ ID NO:3) with extremely high affinity (K_{d}=8 pM) may be used. The dissociation rate constant is relatively fast (0.4 s⁴) because the on-rate of DNA binding proteins is ˜100-fold faster than diffusion control. There also is available data on related DNA sequences that bind with lower affinity (down to K_{d}˜100 nM), and on mutations that modulate binding affinity and specificity. Therefore, using proper signal readouts binding to a battery of DNA sequences may be detected differing in their affinity proportionally to their divergence from TAATTA. If binding to different sequences involves different engHD conformational ensembles (e.g. increasingly compact the tighter the binding), the readout of the sensor could discriminate between alternative sequences. To implement such sensor various high-sensitivity fluorescence readouts (as described below) on an engHD variant that has been purposely destabilized may be employed.

Single-Molecule Signal Readouts

Nanosensors need signal readouts that are high-gain for single-molecule detection and also analog. To achieve both properties implement ratiometric fluorescence signals by trying various approaches using extrinsic fluorescent probes and/or tools for in vivo imaging.

### Extrinsic Fluorescence Methods.

Example methods may include Forster resonance energy transfer (FRET), photoinduced electron transfer (PET) quenching, and excimer pairs. FRET provides a sensitivity range between 0.5 to 1.5 times the characteristic R₀ for the FRET-pair (e.g. from 2.5 to 7.5 nm for common Alexa FRET pairs). Another advantage is that it provides a ratiometric ruler, which reduces ambiguity in signal interpretation. PET quenching is a high-gain method sensitive to long molecular distances because PET capture radii are large (up to 1.5 nm). The formation of excimer pairs between two fluorophores reports on transient close spatial contacts between the two probes. Any of the FRET, PET, or Excimer based read-outs may be employed by placing extrinsic fluorophores on specifically engineered Cys residues in the downhill module of interest.

### In-Vivo Approaches.

For in vivo read-outs, the FRET methods may be employed using several available alternatives. In one example, the bi-arsenate fluorescence derivatives (FlAsH and others) and fusions to variants of the green fluorescent protein (CFP, GFP, YFP, RFP) may be used. For both systems there are multiple colors available, which allow for design of homo- and hetero- (e.g. FlAsH-RFP) FRET pairs. The bi-arsenate derivatives may be implemented introducing the CCXXCC sequence on the exposed face of an α helix of the sensing downhill module. GFP variants may be fused to one of the sensor protein ends.

Multivalent Adhesion

In one embodiment, systems and methods for detection of one or more biological agents are provided. In particular, systems and methods for direct detection of one or more biological agents are provided. In general, the higher the affinity of the sensor protein for a biological agent (e.g., viral spike protein) the lower the limit of detection, and the higher the sensitivity and specificity of the assay. However, direct detection of viral particles without an amplification process (such as that carried out in RT-PCR, other nucleic acid-based tests or ELISA sandwich tests) is very challenging due to the infinitesimal concentrations of the virus (that are sufficient to produce infection, but needed to be detected for diagnosis and screening).

For instance, the viral loads in saliva specimens of COVID-19 patients have been found to be in the range of 10³ to 10⁸ viral particles per milliliter, depending on patient and stage of the infection. These viral loads correspond to concentrations of viral particles ranging from hundred femtomolar (10⁻¹³ M) to about 1 attomolar (10⁻¹⁸ M).

The inventors herein have recognized the above disadvantages and provide systems and methods in order to overcome at least some of the above-mentioned disadvantages. Accordingly, in one example, hierarchical assemblies of an engineered responsive protein or an oligomer of the engineered responsive protein are generated, wherein the hierarchical assemblies have one or more structural properties that resemble a host cell of a biological agent infecting the host cell. Further, the hierarchical assemblies of the engineered responsive protein or the oligomer of the engineered responsive protein (that is, nanoassemblies of the engineered responsive protein) may be constructed based on multivalent adhesion on one or more substrates in order to mimic the host cell. The one or more structural properties include, but not limited to a shape and/or a size of the host cell. In one example, the shape may be based on surface proteins on the host cell that the biological agent may bind to.

In this way, binding affinity is improved, and direct detection of biological agents (without amplification) may be performed with increased specificity as well as sensitivity.

Multivalent adhesion emerges when the interacting particles with compatible shape contain many copies of their binding site so that multiple binding events can take place, sequentially or even simultaneously. To achieve multivalent adhesion, synthetic analogues of host cells (that is, host cell decoys) may be constructed. The host cell decoys may include micron-sized polystyrene beads coated with a desired number of copies of an engineered sensor protein or nanoassembly of the engineered responsive protein.

In some examples, an engineered downhill folding protein (that is, the engineered responsive protein) or an oligomer of the engineered downhill folding protein is coated onto a plurality of substrates at a desired density per substrate to generate hierarchical assemblies of the downhill folding protein or the oligomer, each hierarchical assembly having a shape and/or a size that resemble a host cell of the target to enhance affinity and specificity via engineered multivalent adhesion.

The mechanism for viral detection of the biological agent relies on the ultrahigh affinity and specific adhesion of biological agent (e.g., viral particles) to a microarray of sensor coated beads. Thus, each bead may be functionalized with sensor protein molecules. As discussed above, the sensor protein molecules may be designed to bind specifically to a protein domain of the biological agent, change conformation upon binding, and generate a ratiometric output via a two-color fluorescence reporter system. As a non-limiting example, the engineered responsive protein may bind to the S1 domain of the viral spike protein of SARS-CoV-2 may be and change conformation upon binding, and also, the engineered responsive protein may include a two-color fluorescence reporter system to generate a read-out of the binding indicating detection of viral particles.

In one example, a sensor protein may be constructed that detects the presence of a biological agent (e.g., viral particles) by recapitulating the biomolecular recognition process of the virus and its host cell receptor. In some examples, a structure of a viral protein-receptor complex may be analyzed to identify an interacting domain of the viral protein and the corresponding interacting domain of the host receptor. Further, to achieve multivalent adhesion, synthetic analogues of host cells (cell decoys) including micron-sized polystyrene beads coated with a desired number of copies of our engineered sensor protein.

As a non-limiting example, the atomic structure of the SARS-CoV-2 S protein-ACE-2 receptor complex, reveals that the receptor binding domain (RBD) of the spike protein of SARS-CoV-2 interacts with the peptidase domain (PD) of ACE-2. Binding between these two proteins occurs with an affinity of ˜5 nM that is about 4-fold lower than that of the SARS-CoV spike protein. The two proteins are large and have sophisticated structural patterns. However, the atomic coordinates of the complex show that the binding interface comprises just two, relatively short segments of the PD domain of ACE-2 interacting with the viral S protein, one is an α-helix (802 at FIG. 8A) and the other a β-hairpin (804 at FIG. 8A). The binding interface is defined by a combination of hydrophobic (aliphatic and aromatic side chains) interactions that provide affinity, and a high number of side-chain hydrogen bonds that provide the specificity for binding. A peptide encompassing the α-helix 802 binds to the S protein with an affinity of ˜1 μM. The β-hairpin fills a wedge in between the helix and the spike protein that should enhance binding cooperativity. Accordingly, the helix 802 is identified as minimal high-affinity target for the spike protein (K_{D}˜1 μM), and the two fragments (that is, the helix 802 and the hairpin 804) combined has an affinity (˜10-fold) and specificity booster. These two elements provide the scaffold for various sensor proteins. The advantage of this sensing element including the helix 802 and the hairpin 804 is that it is both simple (two protein fragments that can be efficiently produced in large quantities by solid-phase synthesis) and specific.

In one example, in order to generate the hierarchical assembly of the engineered responsive protein, a statistical mechanics model of multivalent adhesion (also referred to as multivalent adhesion model) may be generated. The multivalent adhesion may be used to engineer a diagnostic test for different conditions of infection (different viral load ranges: diagnostic or screening applications).

For the CoV-2—ACE-2 interaction, in a multivalent adhesion process the effective dissociation constant for adhesion is defined as:

RT in K_{D,AH}=RT In K₀+ΔG_{n̅}

where K₀ is the 0-valency dissociation constant between the two surfaces when no specific interactions are made. ΔG_{n̅} is the average free energy of binding between the two particles, which is obtained from:

$$Δ {G}_{\overline{n}} = \overline{n} Δ G - RT ln [ ( \begin{matrix}{N}_{R} \\ \overline{n}\end{matrix} ) ( \begin{matrix}{N}_{L} \\ \overline{n}\end{matrix} ) \overline{n} ! ]$$

where N_{R} is the number of sensor proteins at the interaction interface and N_{L} is the number of S1 spike protein domains at the interface, ΔG is the binding free energy for the interaction between one sensor protein and one S1 spike protein domain and n is the average number of sensor-S1 interactions made at the interface. K₀ is obtained from the following expression:

$${K}_{0} = \frac{{N}_{R} {N}_{L}}{{M}_{R} {M}_{L}} {v}_{eff}^{- 1}$$

where ν_{eff} is the effective molar volume in which sensor protein-S1 spike protein binding occurs, M_{R} and M_{L} are the total number of binding sites per bead and viral particle, respectively. That is, the effective dissociation constant for adhesion can be computed from the dissociation constant for a single sensor-S1 interaction, the distribution of interacting sites on both virus and bead, and ν_{eff}, which is a parameter that depends on the geometry of the two interacting surfaces.

As estimate of ν_{eff} the value previously obtained for the adhesion of the influenza virus to host cells since influenza and SARS-CoV-2 have similar size and shape (2.7.10⁶ M⁻¹) may be used. For SARS-CoV-2 M_{L} is 74×3, and M_{R} is controllable by the coverage density of the beads with sensor protein (e.g. 1,000 sensor protein molecules). ΔG is obtained from the dissociation constant for sensor protein binding to a single S1 spike domain as ΔG=RT In K_{D}, or −34 kJ/mol for the K_{D}=1 μM of the ACE-2 α-helix of our simplest protein sensor design (design 1 at FIG. 8B). Estimating the interaction surface of the virus to be about one third of its total surface area (similar to the influenza virus), there will be 24 possible spike proteins (8 trimers) to interact with a bead (N_{L}). The average number of interactions between viral particle and bead will be controlled with the coverage density of sensor molecules on the bead (see next section).

Sensor Protein Beads (Alternatively Referred to Herein as SENPROBEs)

SENPROBEs are synthetic cell decoys that result from the functionalization of polystyrene beads with the fluorescently labeled protein sensor and a chemical moiety for their surface immobilization. Sensor beads may attract viral particles by multivalent binding affinity and change their fluorescence readout in response to interactions with the virus. The beads can be functionalized with any desired number of sensor proteins in the range between 100 and 50,000.

Amplification of the binding affinity and specificity to viral particles can thus be engineered by controlling the density of sensor protein molecules coating the beads. For instance, a 1 μm bead (surface area 3.14.10⁶ nm²) with 1,000 sensor proteins will have a density of 1 sensor protein per 3,140 nm², or 8 sensor proteins on the virus reciprocal interaction area (N_{R}). In such configuration an average of ˜4 interactions between bead and viral particle may be estimated. The following will be described with respect to CoV-2; however, it will be appreciated that the methods and systems described herein is applicable to other biological agent detections without departing from the scope of the disclosure.

With this estimate of viral particle-bead interaction and the parameters given above, the dissociation constant for adhesion of the CoV-2 virus to one SENPROBE is about ˜10⁻⁴¹ M. In other words, the beads act as thermodynamic sinks for the virus, so any viral particle present in a clinical sample (even a single particle) will be adhered to a bead, provided that there are enough beads to accommodate them all (about 80 viral particles per bead).

### Construction of SENPROBEs.

In one example, micron-sized polystyrene beads (e.g. Spherotech Inc.) may be used for protein functionalization and surface immobilization. The beads may be functionalized with either amino or carboxylic groups that can be used to covalently link a sensor protein through its free N-terminal amino or C-terminal carboxyl group. The beads may also be used with streptavidin functionalization (sparse coating) that can be used for immobilization to the surface of wells coated with biotin (e.g., available from G-sciences).

Further, in some embodiments, SENPROBE configuration may be performed in three-stages:

First Stage: A full S1 spike trimers may be integrated into membrane nanodiscs (FIG. 10A) (e.g., from Cube Biotech GmbH) to determine the affinity boost caused by the trimer and the bead's maximal loading capacity. FIG. 10A shows an illustration of SARS-CoV-2 spike trimer inserted into nanodisks or solubilized in micelles and an outline of procedure to produce spike-trimer decorated liposomes.

Second Stage: Further, 100 nm liposomes may be decorated with spike trimers via detergent-mediated reconstitution (FIG. 10A) to be used as synthetic viral mimics (similar size, shape and density of spike proteins). Briefly, liposomes (prepared by sonication followed by extrusion) may be mixed with increasing amounts of detergent to induce the lamellar to micellar transition (the solution becomes optically transparent); then the spike trimer solubilized in detergent micelles (Cube Biotech GmbH) may be added at the required lipid to protein ratio. Proteoliposomes are formed by progressive removal of detergent via successive additions of SM-2 Adsorbent Bio-beads (Bio-Rad). The spike-trimer decorated liposomes may be used for the configuring multivalent adhesion (ν_{eff}, N_{R}, N_{L} and n̅), and to determine the desired sensor coating density.

Third Stage: Full viral particles (gamma inactivated on BSL1 and active on BSL-3, both may be obtained from the NIAID BEI Resources Repository) may be used for the fine characterization of multivalent adhesion and final optimization.

Validation, evaluation and benchmarks. Biomarker (spike trimer, proteoliposome, virus) adhesion may be monitored by fluorescence on surface-attached (fluorescence cell imager) or free (flow cytometry at UCM-IMF) beads. The benchmark is the lowest viral loads of COVID-19 saliva samples (10′ per mL). Spike-trimer liposomes may be characterized structurally by cryo-electron microscopy. FIG. 10B shows an example high-throughput fluorescent bead imaging using a multimode reader/cell imager. Full 96-plate, well image (2×, left) and magnified region (20×, right) with fluorescence FACS calibration beads (4 colors) are shown. Automated measurements of full 96, or 384 well plates and intensity analysis of each bead, using cell counting algorithms (single bead diagnostics), may be completed in <10 mins.

### Fluorescence Readout

Another element is the transducer mechanism to convert the binding of the virus to the SENPROBE beads into a signal that can be easily readout. In one example, a fluorescence signal associated to single bead imaging may be used. Fluorescence has very high sensitivity to the extent that it can be used to measure the emission of single molecules. In one example, single-molecule fluorescence spectroscopy applied to measure protein conformational changes. Further, fluorophores in the visible range may be used and the detection of individual protein molecules may be performed by fluorescence. Fluorescence can be measured immediately after sample application and very quickly (˜1 minute) on a simple fluorometer with well-plate reader (thus resulting in extremely fast testing).

In some examples, the SENPROBEs may be immobilized on the bottom of the well and inverted excitation and fluorescence detection may be used. In such a configuration, all the fluorescence originates from the surface itself so that measurements on highly absorbing samples are possible with minimal scattering. In addition, a two-color fluorescence readout may be incorporated for ratiometric (internal calibration) determination. Three fluorescence reporters may be used to convert the binding of engineered mini-protein to the viral S protein into signals that can be interpreted quantitatively to determine the viral content of the sample.

Design 1: An example approach is based on the 34-residue α-helix of ACE-2 as minimalistic protein sensor and a single fluorophore that undergoes excited state induced protein transfer (ESIPT) as reporter. ESIPT fluorophores change their properties depending on the polarity of the environment (solvatochromism): e.g. from water exposed (when the sensor protein is free) to buried inside a protein complex (when a viral particle binds to the sensor protein). A fluorogenic dye 6-N,N-dimethylamino-2,3-naphthalimide, (6DMN) may be used which changes its quantum yield by 120-fold when transferred from water to a hydrophobic environment, and also experiences a shift in emission from orange (λₘₐₓ˜595 nm) in water to cyan (λₘₐₓ˜510 nm) in non-polar solvents(20). Another option is to use 2-(2-furyl)-3-hydroxychromone (FHC), which has dual emission (λ1ₘₐₓ˜430 nm, violet, and λ2ₘₐₓ˜530 nm, green) in which the relative intensity of the bands is highly sensitive to the polarity of the environment. Both dyes can be synthesized as F-moc aminoacids and incorporated to synthetic peptides during solid-phase synthesis.

The fluorophore will be incorporated into position 16 (replacing histidine) (FIG. 8B). This position is completely solvent exposed in absence of virus and becomes covered by the viral S-protein upon complexation. Computer modeling indicates that the fluorophore will not interfere with the binding process (there is enough space to fit it in), but rather enhance it through added hydrophobic interactions. The formation of the complex may drastically change the environment of the fluorophore providing a sensitive, very high gain readout (the fluorophore molecules in sensor protein molecules that are bound to the virus will change color (blue or red shift) and increase emission intensity by up to 120-fold. The advantages of this option are simplicity of design and very high sensitivity (it is much easier to detect very low fluorescence without any background than a small change in intensity of a large signal).

Design 2: The second approach is based on a hybrid sensor that contains both the α-helix and β-hairpin joined with a flexible linker to make a fusion peptide that recapitulates the entire viral S-protein binding site (FIG. 8B). In absence of the virus the sensor protein will be split in two halves (helix and hairpin) because they do not have sufficient interactions with each other to stabilize the complex structure. However, the two halves will come together upon binding of the viral S-protein, which provides the interface to stabilize the ternary complex. In such hybrid system the binding event produces a conformational change on the sensor protein that is used as signal transducer to the fluorescence readout.

An example of the hybrid sensor protein including the linkers and a reengineered sequence eliminates hydrophobic residues on the face opposite to the binding interface to minimize non-specific binding and/or aggregation. The two fragments can be synthesized separately by solid-phase methods, and then joined enzymatically using the sortase chemistry (the two fragments incorporate the sequences at their ends recognized by sortase).

The hybrid sensor may exhibit higher specificity, and the use of a conformational fluorescence reporter minimizes any potential binding impairment due to fluorophore interactions.

As fluorescence readout, any of two different two-color reporters may be used. Design 2a (FIG. 8B) is based on the contact quenching of oxazine fluorophores (emit in the red, i.e. Atto 655) by photoinduced electron transfer (PET) from a tryptophan residue in the protein. PET occurs when the electron acceptor (oxazine fluorophore in its excited state) is within molecular contact (<1 nm) of the donor (tryptophan). At distances shorter than 1 nm, the fluorescence is fully quenched (no emission) whereas at longer distances the phenomenon does not take place. Atto 655 will be incorporated into the helical segment (via maleimide chemistry onto a cysteine placed instead of the phenylalanine in position 22). When the viral S protein binds to the sensor's α-helix, the β-hairpin will fold over inserting itself into the cleft formed by the complex of the helix and the viral S protein to form the ternary complex. In the ternary complex the natural tryptophan residue (position 10 on the hairpin) will be at PET distance of Atto655 to quench its fluorescence. The design also substitutes a natural tryptophan on the helix (position 30) to tyrosine to remove a potential (binding unspecific) PET donor. The advantages of a PET readout are that the phenomenon has very short distance dependence (1 nm), which increases its molecular specificity, and a binary response that maximizes the signal gain (on or off). The disadvantages are: 1) it is not ratiometric, and 2) the positive result (binding of the viral particle) results in a loss of signal, which makes it more susceptible to false positives. Further, to reduce non-ratiometric output and false positive, a two-color approach may be used in which a second dye (green) is incorporated to the sensor protein in a position insensitive to the binding of the virus so that it can be used as internal calibration. In this configuration the sensor protein includes both the α-helix and the β-hairpin to increase its binding/signal specificity. The helical peptide will be labeled with Atto 655 (via maleimide chemistry onto a cysteine residue placed instead of the phenylalanine in position 22). The internal control fluorophore will be attached to the N-terminus of the α-helix during solid-phase synthesis.

Design 2b (FIG. 8B) is a FRET based sensor with two fluorophores, one on each segment of the hybrid sensor protein. The two fluorophores are chosen to be a Forster resonant energy transfer pair (e.g. A488 and A594). In the absence of virus, the two halves are split and the distance between the fluorophores is longer than the characteristic R₀, which results in low FRET efficiency (higher green intensity). Upon binding of the virus, the ternary complex is formed, making the distance between the fluorophores shorter than R₀ and thus an increase in red emission over green. The advantages of FRET are its intrinsically ratiometric nature (the signal depends on the ratio of the red and green intensities), and its distance dependence, which makes the signal (FRET efficiency change) work as molecular ruler.

Turning to FIG. 9, it shows a schematic illustration of an example multivalent adhesion approach for detecting an example biological agent, such as SARS-Cov-2 in an one-step fluorescence read-out. Micrometer-sized polystyrene beads (larger spheres) are coated at a designed density with an engineered peptide binding moiety (protrusions from larger sphere) that recreates the binding site on ACE2 that is recognized by the spike protein of SARS-CoV-2. The peptide binding moiety is tagged with a fluorescent moiety (tips of protrusions) for fluorescence-based detection of binding events from viral particles to form a sensor peptide. The fluorescent moiety contains a two-color readout for accurate quantitative ratiometric detection of viral particles. The first color is emitted from sensor peptide molecules not bound to the virus (lighter tips of protrusions) and the second color (darker tips of protrusions) is emitted upon adhesion of the sensor peptide to functional (infective) viral particles.

The sensor peptide-containing beads are immobilized on the transparent surface of a sample holder (e.g., the bottom of a well of a multi-well plate) in a sparse arrangement by, for example, chemical attachment to the surface via a biotin-streptavidin linkage.

### EXAMPLES

The following examples are provided to better illustrate the claimed invention and are not intended to be interpreted as limiting the scope of the invention. To the extent that specific materials or steps are mentioned, it is merely for purposes of illustration and is not intended to limit the invention. One skilled in the art may develop equivalent means or reactants without the exercise of inventive capacity and without departing from the scope of the invention.

### Example 1: BBL-Tandem Responsive Proteins

BBL is a 40-residue fragment of the E2 subunit of the 2-oxoglutarate dehydrogenase multienzyme complex of Escherichia co/i, which comprises a peripheral E1-E3 subunit binding domain. BBL has been identified as a globally downhill folding protein. That is, BBL has no free energy barrier to folding in any thermodynamic condition. The thermal unraveling of BBL tertiary and secondary structure is broad and weakly coupled, with different structural/spectroscopic techniques such as CD and differential scanning calorimetry displaying different behaviors that highlight a low degree of cooperativity. The analysis of the BBL thermal unfolding transition at atomic resolution using nuclear magnetic resonance showed a highly heterogeneous unfolding process in which individual atoms exhibit notable differences with a distribution of melting temperatures (Tm) that spans 60 K. These results ruled out the possibility that BBL folded-unfolded through a binary transition and was the first experimental identification of a downhill folder. The ensemble population in BBL shifts from fully native to fully unfolded by gradual melting of its structure. Analysis of the thermal unfolding data with a structure-based statistical mechanical approach yielded no barrier heights to folding under any condition, a globally downhill folding scenario. These earlier findings have been later confirmed by a wide range of techniques, including atomic resolution equilibrium unfolding experiments, multiprobe laser T-jump kinetics, differential scanning calorimetery, single-molecule fluorescence spectroscopy and large-scale molecular dynamics simulations.

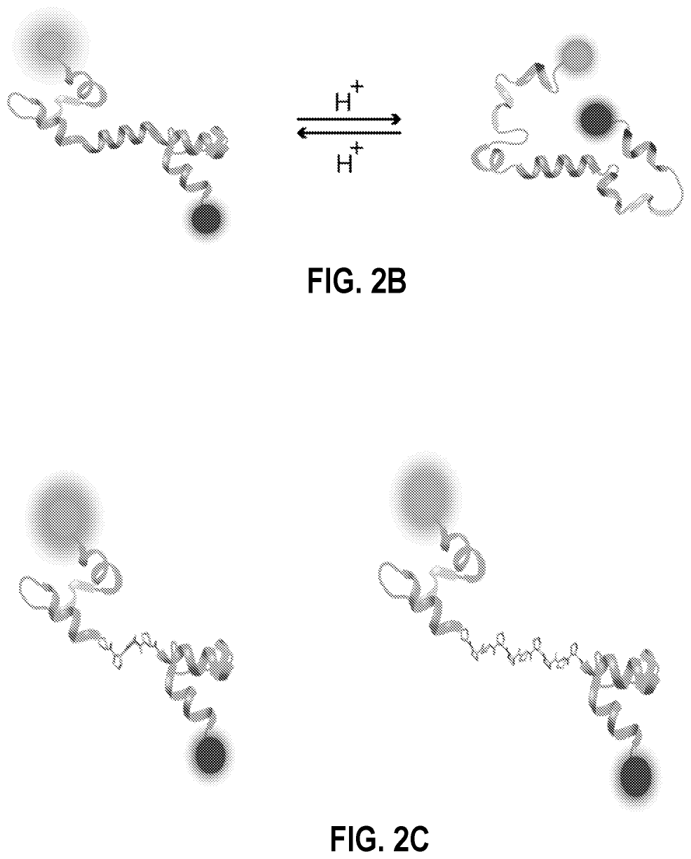

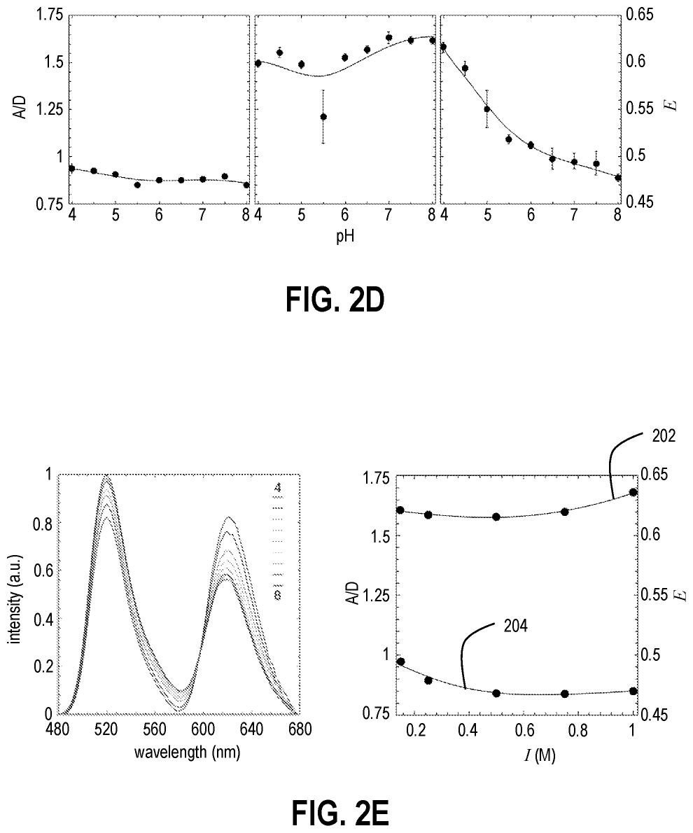

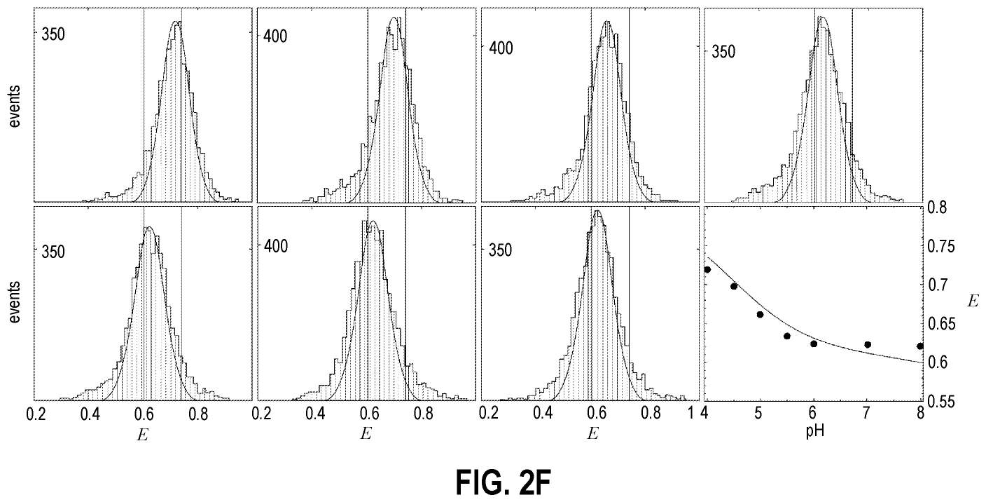

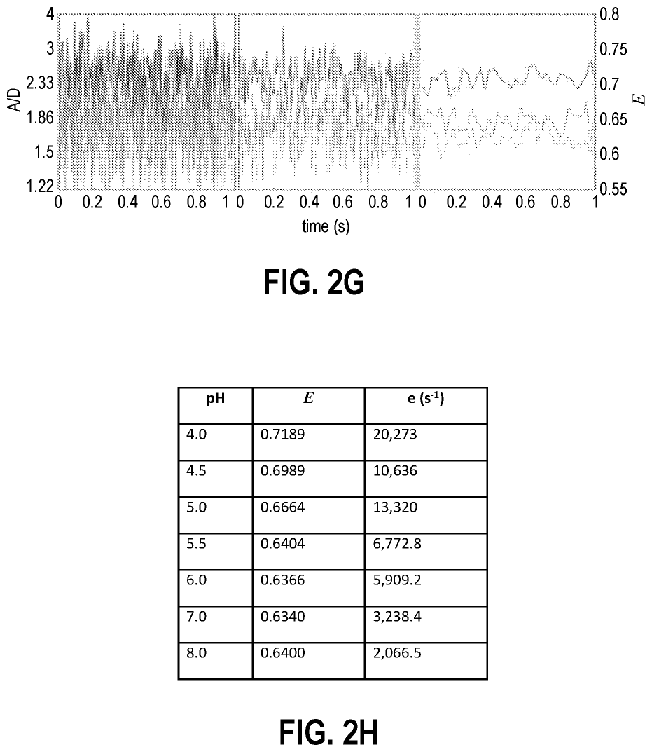

The BBL folding-unfolding behavior is thus a paradigm of conformational rheostat, a mechanism that was postulated to be of functional significance, particularly when coupled to the folding upon binding of intrinsically disordered proteins (IDPs). BBL also naturally exhibits gradual unfolding via mild acidification with an apparent pKₐ of approximately 5. The reason behind such sensitivity to pH in the 8-4 range is the presence of two buried histidines (H13 and H37), which are involved in several tertiary interactions in the native BBL structure that bring the two helices together, as shown in FIG. 2. pH titration using NMR rendered pKₐ values of ˜6.6 for H13 and ˜5.6 for H37. The pKₐ of H37 is 1 pH unit lower than that of free histidine, indicating that the native structure of BBL strongly favors the deprotonated state of H37. Additionally, BBL has five negatively charged residues (three E and two D) that could also contribute to the unfolding of BBL at more acidic conditions. Electrostatic repulsion at lower pH could be a source for long-range interactions that destabilize the protein. A calculation of the net charge on the protein considering a simple scenario of invariant pKₐ of the ionizable amino acids on the protein (of which there are 14), indicates that the net charge of BBL should increase from 2.3 at pH 7 to 8.5 at pH 3.

A previous approach by Cerminara et al titled “Downhill Protein Folding Modules as Scaffolds for Broad-Range Ultrafast Biosensors” in Journal of the American Chemical Society, 2012, 134(19), pp. 8010-8013, which is incorporated by reference herein in its entirety, demonstrated BBL's folding sensitivity to pH as gradual (rheostatic) conformational transducer of pH and ionic strength. Therein, it was shown that the coupling between proton binding and the gradual (un)folding of BBL resulted on a broadband conformational transducer with sensitivity to pH in the 4-8 range. Further, it was shown that the unfolding upon binding response of BBL at room temperature was ultrafast (<20 s), owing to its microsecond folding kinetics. At mildly acidic pH BBL's folding proved to be extremely sensitive to ionic strength, because the ions in solution shielded the electrostatic repulsions caused by the excess of positive charge in that pH range. This behavior was exploited to implement a bifunctional pH-ionic strength transducer based on the natural BBL protein, and an engineered BBL version that replaced all the negatively charged residues in BBL (D and E) by its uncharged counterparts (N and Q) to result on a protein with high positive charge at any pH below 9 (BBL+). BBL+ folding was insensitive to pH (always unfolded), but sensitive to ionic strength in broadband fashion (over 3 orders of magnitude in ionic strength). The combination of BBL and BBL+ thus afforded the possibility of determining both ionic strength and pH from the conformational changes of the two protein. This work demonstrated the potential of coupling downhill (un)folding to binding to implement high-performance conformational transducers capable of broadband and ultrafast responses.

However, the pH-induced conformational changes of BBL are very local, and accordingly their monitoring needs the use of an optical technique sensitive to the backbone conformation, such as CD. CD is based on the differential absorbance of circularly polarized light, and hence it is low sensitivity, extremely reliant on highly transparent samples, and, most importantly, not specific because every protein and nucleic acid have comparable CD signals. The inventors herein have identified the above-mentioned issues, and have recognized that in order to convert conformational rheostatic transducers into functional biosensors, the transducers may be effectively coupled to high sensitivity and specificity readouts (i.e., fluorescence) that could be used for real-time monitoring in living cells. An extremely high sensitivity readout is also essential to demonstrate that rheostatic conformational transducers can give rise to biosensors capable of producing analog (quantitative) signals at the single-molecule level. This property, offers the highly desirable and unique feature of extreme biosensor miniaturization to reach real-time monitoring with nanoscale resolution.

Accordingly, in one example, a BBL-based biosensor that can effectively convert the pH sensitivity of BBL into a high-sensitivity ratiometric fluorescence readout is provided. Further, an analog real-time reporting of pH from a single-molecule device is demonstrated below.

Design of a Conformational Rheostat Amplifier

In one example, the pH unfolding of BBL may be converted on a large enough change in conformation so that it results in a clear change in FRET efficiency. The BBL is a very small domain (about 40 residues) that remains highly compact upon pH unfolding. In fact, the BBL native 3D structure (pdb:2CYU) shows the protein ends pointing in opposite directions. Upon pH denaturation, the distance between the two ends of BBL becomes even shorter, which suggests that upon pH denaturation BBL remains compact but the two helices swivel thereby shortening the end-to-end distance to ˜2 nm. To produce a high sensitivity fluorescence readout based on FRET, fluorophores in the visible range may be used with very high quantum yields and strong absorbance. These properties result on FRET pairs with R0 values (distance at which there is 50% FRET efficiency) around 6 nm, that is, sensitive to changes in the 3 to 9 nm range due to the six-power dependence of the FRET efficiency with the distance. Therefore, conversion of the pH-induced changes in end-to-end distance of BBL into a suitable range for high-sensitivity FRET measurements requires the design and implementation of an amplifier of the conformational change.

In one example, an engineered protein may comprise two BBL domains arranged in a tail to head tandem connected by a rigid structural spacer of potentially adjustable length. The combination of two BBL domains aims to amplify the magnitude of the 0.5 nm change that takes place in one domain. The spacer aims to increase the total end to end distance to shift the conformational changes closer to the maximum sensitivity range (close to R₀). In some example, the rigid spacer may be either a polyproline stretch, or a sequence designed to continuously connect the second helix of the first BBL with the first helix of the second BBL in the tandem (see FIGS. 2B and 2C). Both types of rigid spacer may be made shorter or longer by simple extension of the sequence, providing an effective tool to optimize the average distance between the fluorophores so that is longer than R0 at neutral pH (both BBL domains maintaining the native helix orientation and low FRET efficiency), and shorter at acidic pH (swiveling of the helices and high FRET efficiency). Based on the above, in one example, an engineered protein composition may include three BBL-tandem scaffolds: one with an alpha-helix-forming sequence as spacer (B-hel-B), and two with polyproline stretches of 4 or 10 residues (B-P4-B and B-P10-B). In one example, the helix spacer in B-hel-B may be 12 residues long (FIG. 2B). Its sequence was designed to form a highly stable α-helix using the program AGADIR. Given the structural parameters of α-helices (0.15 nm rise per residue and helical pitch of 0.55 nm), the helix spacer should increase the end-to-end distance in the tandem by about 1.8 nm.

In another example, a spacer may include a stretch of prolines, which may form a polyproline II helix, a type of protein secondary structure that is almost fully extended and rather rigid. A left-handed polyproline II helix (PPII) is formed when sequential residues all adopt backbone dihedral angles φ and ψ of roughly ˜75° and 145°, respectively, and have all their peptide bonds as trans isomers. A full PPII helical turn requires 3.0 residues with 0.31 nm rise per residue (effective helical pitch of 0.93 nm), and hence it is rather extended. The PPII conformation is the preferred conformation of a stretch of prolines in aqueous solution, but it is also common in short segments of proteins and polypeptides in which prolines alternate with other amino acids. Additionally, a more compact right-handed polyproline I helix (PPI) can also be formed when sequential residues all adopt backbone dihedral angles of roughly −75° and 1600 and have their peptide bonds as cis isomers (3.3 residues per helical turn and 0.17 nm rise per residue). Because of these properties, polyproline chains have been used as a “molecular ruler” in structural biology and biophysics, e.g., to calibrate FRET efficiency measurements. Particularly, two polyproline spacers, one with four prolines and the other with ten, may result in end-to-end distance extensions of 1.2 nm and 3.1 nm, respectively, when in a rigid PPII conformation (FIG. 2C).

Additionally, the three BBL tandem designs incorporated the sequence CKKNNDAL at the N-terminal of the protein, in order to facilitate fluorophore conjugation and dynamic isotropic averaging of the fluorophore.

Converting pH into a FRET Signal: Bulk Fluorescence of BBL-Tandems

In one example, to implement the FRET readout, the various BBL-tandem constructs on the cysteines placed at their ends are labelled with with Alexa 488 and Alexa 594. After purification of the doubly labeled sample, a steady-state fluorescence experiments at various pH values were performed to determine to evaluate the amplification of the conformational rheostatic change of BBL. FRET efficiency (E) was calculated after normalizing the spectra from the intensity of the acceptor band divided by the total.

The results from these experiments are summarized in FIG. 2D. We observed that the FRET efficiency of B-P4-B was the lowest of the three and that of B-P₁₀-B the highest, consistently with their long and short spacers. However, neither of these constructs convert the local conformational changes of the BBL domains into a FRET change. B-P4-B has a completely flat profile with E˜0.47. The B-P₁₀-B exhibits FRET efficiencies around 0.6, but it is also essentially unresponsive to pH. In addition, this protein exhibited some propensity to aggregate around its pI (˜5.5), resulting in large errors around that pH region (see middle panel in FIG. 2D).

The FRET values for the two tandems with polyproline linkers are qualitatively consistent with the difference in the spacer's length. However, the difference in E between B-P₄-B and B-P₁₀-B corresponds to an additional extension of ˜0.6 nm for the latter, whereas the increase for a rigid PPII should be almost 2 nm. Of course, this calculation assumes that the PPII and its linkages to the two BBL domains are all completely rigid. It is very likely that the connections with the two BBL domains are rather flexible, resulting on a more collapsed globular structure with shorter end to end distance, as we see experimentally. However, the most important result is that the polyproline linkers do not effectively amplify the local conformational changes of the BBL domains with pH.

In contrast, B-hel-B exhibits a significant change in E as a function of pH in bulk. In this protein, E changes monotonically from ˜0.48 at pH 8 up to ˜0.62 at pH 4 (FIG. 2D). The total change is similar in magnitude to the difference in E of the two polyproline spacers, corresponding to a global expansion of ˜0.6 nm. The change is most pronounced between pH 6 and 4, but it does continue up to pH 8, albeit in weaker fashion. The change in E of B-hel-B is equivalent to a change of 1.6 in the acceptor/donor ratio, which is comparable to the total change in signal of many commercial FRET-based sensors. Additionally, there is a slight red shift in the acceptor emission maximum of B-hel-B as the pH is lowered. Such an effect is however not observed in the donor emission. This could be due to the acceptor encountering a more hydrophobic environment. Based on these results, the coupling of the second helix of BBL1 with the first helix of BBL2 via a helical spacer works as an effective amplifier of the local conformational changes that the BBL domains experience with pH. Hence, B-hel-B is a suitable candidate for fluorescence detection of pH changes in the 8-4 range.

Another important factor to consider for the suitability of B-hel-B as fluorescence pH biosensor is the potential dependence on ionic strength. The wildtype BBL protein did refold from the acid denatured state by increasing ionic strength. The persistence of such an effect on B-hel-B could hamper the interpretation of the fluorescence changes in terms of pH change. This possibility was tested by performing bulk fluorescence measurements on B-hel-B at the two extreme pH values, and as a function of ionic strength in the 0.1 to 1 M range. The results showed very little ionic strength dependence for B-hel-B at both extreme pH value (See FIG. 2F). At pH 4, B-hel-B is essentially ionic strength insensitive up to 0.8 M, exhibiting a minor increase in E above that point (FIG. 2E). In contrast, at pH 8 the response is flat above 0.5 M, and there is a minor effect at the lowest ionic strength. Overall, the fluorescence sensitivity to pH and lack of sensitivity to ionic strength indicate that B-hel-B is a good fluorescence pH biosensor and an excellent candidate for exploring the implementation of single-molecule analog pH detection.

Single Molecule Confocal Microscopy in Free Diffusion of B-Hel-B

Before performing single-molecule fluorescence experiments on the B-HelB pH biosensor, it is important to consider the expected differences between a conformational switch (two-state folding) and a conformational rheostat (downhill folding) in the context of single molecule-FRET experiments. In the context of the detection of a limited number of photons from an individual molecule, the actual output of the experiment depends on the interplay between the experimental binning time (TB), which sets the time resolution, and the folding relaxation time (tF) of the protein. In a two-state folding scenario where a protein folds over a significant free-energy barrier (>3 RT), individual molecules populate either the folded or the unfolded state. When TB<<tF, the FRET efficiency histogram shows two peaks with amplitudes dependent on the folding equilibrium constant. Since the conformational dynamics within each state are much faster than the overall folding relaxation, both peaks should have shot-noise limited widths. However, when TB>>tF molecules undergo multiple folding-unfolding events during the observation time, and thus the FRET efficiency histogram becomes a single, shot-noise limited peak at a position reflective of the population weighted average. In a one-state (downhill) folding scenario, the FRET efficiency histogram will have a unimodal distribution at all conditions. When TB>>tF the FRET efficiency histogram will be shot noise limited, and indistinguishable from two-state folding. The difference arises when TB<<tF, which for a downhill folder will still result on a unimodal distribution, but in this case with potentially additional width over the shot node, revealing the actual conformational distribution.

The other important factor to consider for single-molecule experiments is the signal to noise of the experiment, as a limited number of photons per unit of time may be detected. The key parameter is the photon count rate that is obtainable from an individual molecule. In optimal conditions and using special photoprotection cocktails to reduce fluorophore blinking and bleaching, count rates of 150-250 photons per millisecond from one molecule may be obtained. Using the shot noise equation, these count rates convert into a determination of the FRET efficiency with ±0.057 shot noise accuracy for measurements made on a single molecule with E=0.5 and 0.5 ms time resolution (TB).

A conformational switching device (biosensor) can produce an analog readout by either performing the measurements using ensemble averaging (i.e. bulk measurements) or by time averaging of one molecule, or by any combination of the two. In this example, to distinctly demonstrate that the B-Hel-B pH sensor produces analog pH readouts at the single-molecule level fluorescence measurements of individual molecules may be performed with sufficiently short time-resolution to avoid systematic time averaging (TB<<tF) and with enough photons (count rates) to reduce the shot noise in the determination of E to levels below the overall signal change due to pH. For this purpose, confocal single-molecule FRET spectroscopy was used in a custom-built confocal fluorescence microscope. In particular, sm-FRET measurements on B-Hel-B in free diffusion experiments at various pH values in the 8.0 to 4.0 range. The results of these experiments are presented in FIG. 2F. In these conditions, count rates from freely diffusing molecules were obtained that allowed to bin the experimental data into 0.5 ms bins using a photon threshold of 75. Donor only bursts were removed using cumulative Poisson distribution function as shown in supporting information, a statistically very stringent test to eliminate any bursts coming from molecules without an active acceptor. Data from thousands of molecules were collected and calculated E for each single-molecule photon burst. FIG. 2F shows the resulting FRET efficiency histogram at the various pH values. The figure panels also shows a curve representing the width of the FRET histogram expected from shot-noise over the experimental mean FRET value given the photon threshold.

The first observation is that the single-molecule experiments recapitulate the average E as a function of pH measured in bulk. This is shown in the last panel of FIG. 2F, in which the average E from the FRET efficiency histograms is compared to the E curve measured in bulk. Actually, the datapoints shown in that last panel correspond to the average signal from a few thousands of molecules measured with 0.5 ms time resolution relative to the steady-state bulk signal. The second observation is that the FRET efficiency histograms are always unimodal and have a maximum that gradually shifts from lower E at pH 8.0 to the highest value at pH 4.0. Particularly, the population moves monotonically from a mean FRET of 0.72 at pH 4 to 0.60 at pH 8. The standard deviation from shot noise was about 0.05 (0.0520 with 4,257 bursts for pH 4.0 and 0.0558 with 6,428 bursts for pH 8.0). These results are an indication that the protein changes gradually in analog fashion, or alternatively that it changes conformation due to histidine protonation so quickly that the histogram is time-averaged as discussed in the previous paragraphs. Given that the time resolution of the experiment is already 0.5 ms, the second reason is extremely unlikely. Moreover, comparison of the histograms with the expected shot noise (black curves in FIG. 2G) shows that the experimental distribution is broader, particularly for the higher pH values. The extra width on the histograms suggests that the conformational dynamics of the protein are not too much than the time resolution (i.e. >>1/(0.5 ms) or 2,000 s⁻¹).

To further investigate this issue, a kinetic analysis of the sm-FRET data was performed. Resolving biomolecular dynamics using sm-FRET experiments through FRET efficiency histogram analysis has practical limitations. In sm-FRET experiment, the probability that a photon is emitted by either the donor or acceptor depends on the inter-dye distance. Thus, the pattern of the color of photons contains information about the inter-dye distance, which is in turn reflects the conformational dynamics happening in the biomolecule. A likelihood analysis method may be performed to extract kinetic and dynamic parameters from the sequence of the color of photons and inter-photon times. GS-MLA model calculates the likelihood that a given set of parameters of a predefined model describes the observed time-stamped photon trajectory. This likelihood function is then maximized to obtain the parameters that best describe the data. In this case, a kinetic model that represents the conformational dynamics of a protein is used as diffusion on a 1D free energy surface. This type of kinetic modelling is ideally suited for the analysis of minimally cooperative protein (un)folding transitions and hence to distinguish between two-state and downhill folding. The same type of analysis should be able to distinguish between a scenario in which the coupling between protein (un)folding and histidine ionization produces a shifting unimodal (analog) or bimodal (binary) distribution. It will also provide the time response of the biosensor from the slowest eigenvalue of the diffusive rate matrix. The results obtained with this method for the B-Hel-B photon trajectory data as a function of pH are summarized in FIG. 2H.

Interestingly, the kinetic analysis indicates that the (un)folding coupled to histidine ionization of B-Hel-B is very fast at the highest pH values (sub-milliseconds), and gets even faster as pH becomes more acidic (about 50 μs at pH 4.0). As we discussed above, these results imply that the response of the biosensor is limited by the proton (ligand) concentration at the highest pH values, and then speeds up as pH drops. However, the acceleration in response is of only a factor of ˜10 for 4 orders of magnitude in [H+], which suggests that at the lowest pH values the biosensor's time response is limited by the folding rate constant of B-Hel-B. The latter is consistent with previous work in which the time response of a single BBL domain was measured by laser-induced temperature jump kinetics. These sm-FRET experiments and analysis demonstrated that B-Hel-B changes its conformational ensemble gradually in response to pH, and that such gradual changes get efficiently converted onto a change in fluorescence signal (FRET efficiency) that operates as an analog single-molecule fluorescence biosensor of pH. Furthermore, the results shown in FIG. 2H also demonstrate that the time response of this biosensor is ultrafast: from 0.5 ms at pH 8.0 to 50 μs at pH 4.0.

Recording pH from Single Molecule Fluorescence: Signal to Noise Analysis from Stochastic Simulations of B-Hel-B.

The experiments described in the previous section demonstrated that B-Hel-B is a fluorescence biosensor for measuring pH that can operate in analog fashion at the single-molecule level. However, in practical terms, it is important to determine what are the specific properties and limits of such sensor, particularly in terms of signal to noise, pH accuracy and time response. Knowing these properties is essential to ascertain the potential usability of the biosensor as a single-molecule pH sensing device. To tackle this key question, a series of stochastic simulations were performed based on the sm-FRET experimental data that we obtained for B-Hel-B (see previous section). The sm-FRET data contains bursts of 0.5 ms because they come from free diffusion experiments. However, longer time recordings may be simulated and the effects of signal averaging (digital filtering) by stochastically appending bursts from different molecules into a simulated compounded trajectory. Such trajectories simulate data acquisition streams for different time averaging and number of molecules. These simulations show that for a time resolution of 0.5 ms, the simultaneous recording of a total of approximately 50 molecules is needed to produce a E signal with an accuracy of 0.01 standard deviation, whereas with 5 molecules the signal to noise decreases by a factor of 4 (shot noise of 0.04). As the simulations show, given the limited maximum signal of the B—Hel-B sensor (FIG. 4.6), distinguishing pH within 1 unit in the 6.00 to 4.00 range requires using a biosensor with at least 50 molecules. Alternatively, the simulation predicts that one could obtain the same signal to noise recording from a single molecule provided that its behavior as averaged over 100 ms.

Long Fluorescence Recordings from Single Molecules: TIRF Microscopy on Vesicle Immobilized B-hel-B.

Further, the recording of the fluorescence signal of B-Hel-B single molecules over long times (seconds) were performed to demonstrate the analog single-molecule sensing of pH in times and conditions relevant to its application as a biosensor. For this purpose, TIRF microscopy was used for the individual visualization of multiple B-Hel-B molecules while immobilized to the surface. For the immobilization B-Hel-B was encapsulated into synthesized biotinylated liposomes that were subsequently tethered to the surface via bioptin-streptavidin-biotin linkages. The liposome encapsulation was performed at specific pH, so that the liposome will a pH constant environment to record the B-Hel-B signal. Encapsulation allows for the controlled environment of the single-molecule biosensor and for a better mimic of the biologically relevant environments and conditions. TIRF uses very mild illumination and permits the simultaneous imaging of multiple molecules present in the field of view (typically a few microns in each dimension using a 100×TIRF objective). The mild illumination conditions of TIRF together with the immobilization allow for the observation of single molecules over much longer periods of time (seconds), unlike free diffusion measurements using confocal microscopy (about 1 ms), albeit with lower temporal resolution. It has additional advantage of better signal to noise ratio owing to the fact that the excitation volume spans over a very narrow depth (about 100 nm) of the evanescent wave. This factor greatly reduces the fluorescence background and hence produces high contrast visualization of single molecules during long times.

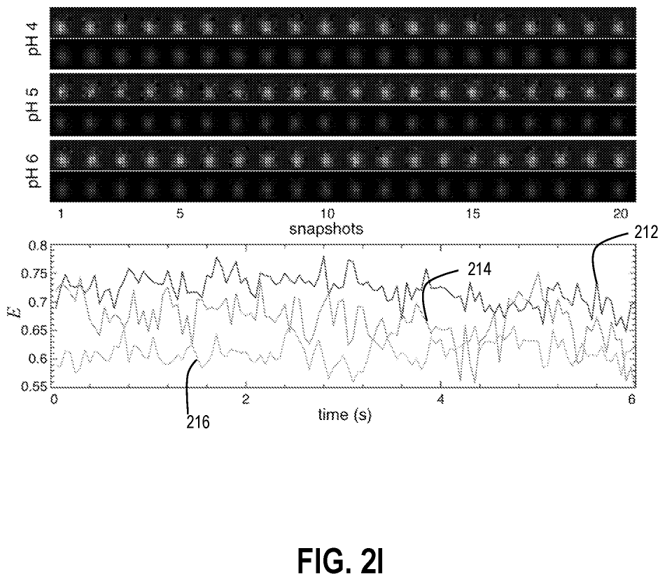

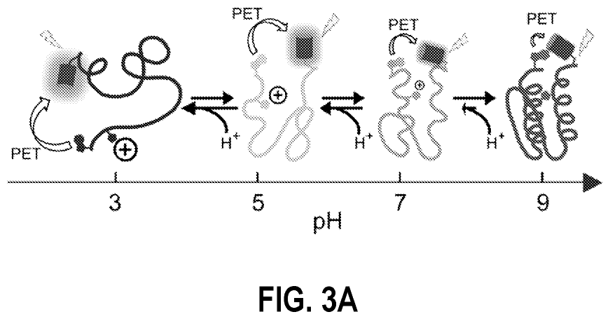

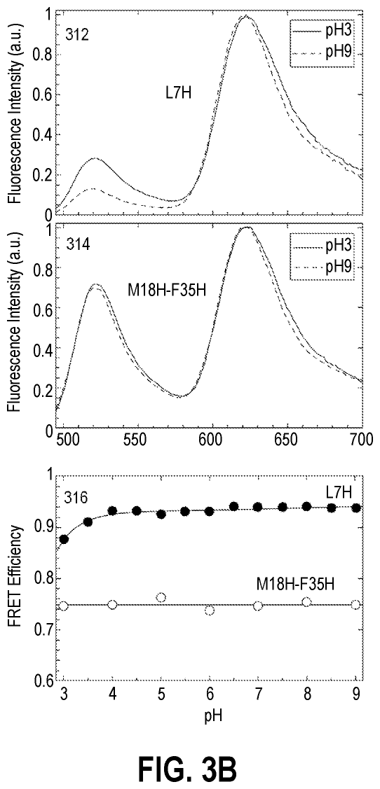

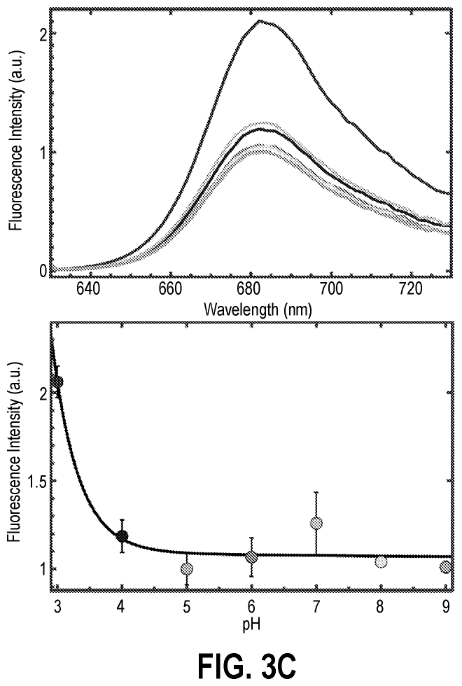

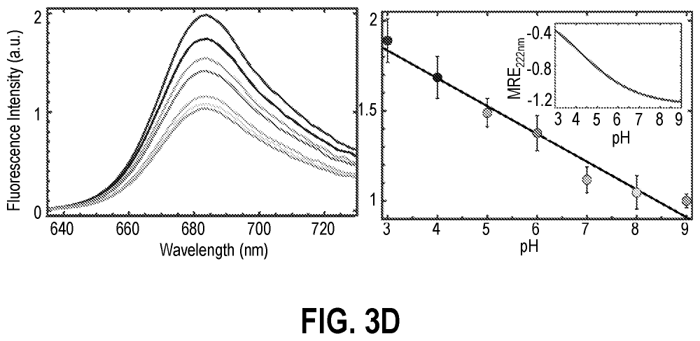

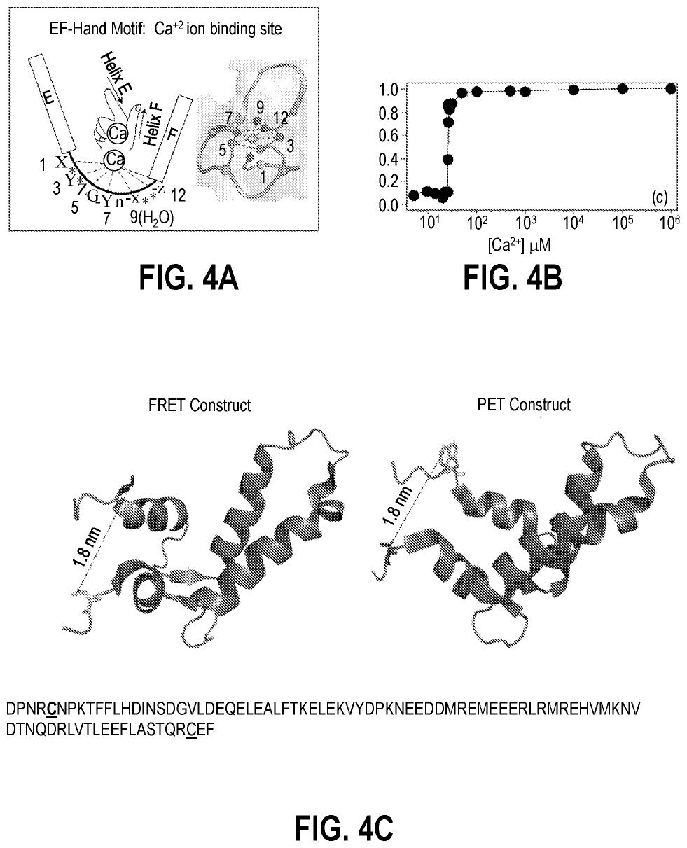

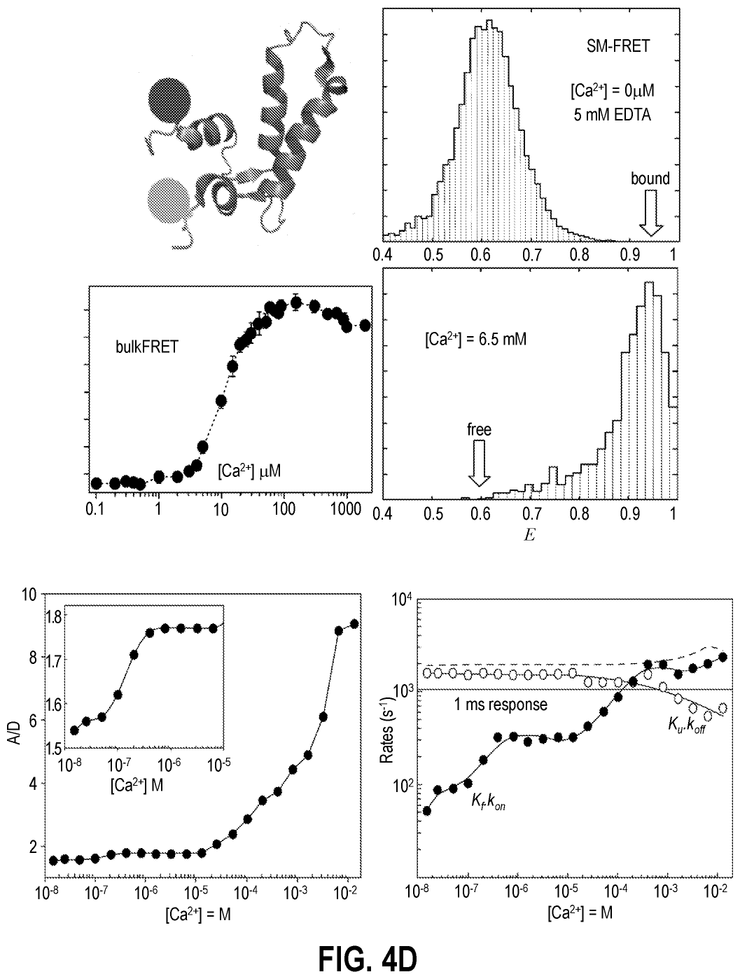

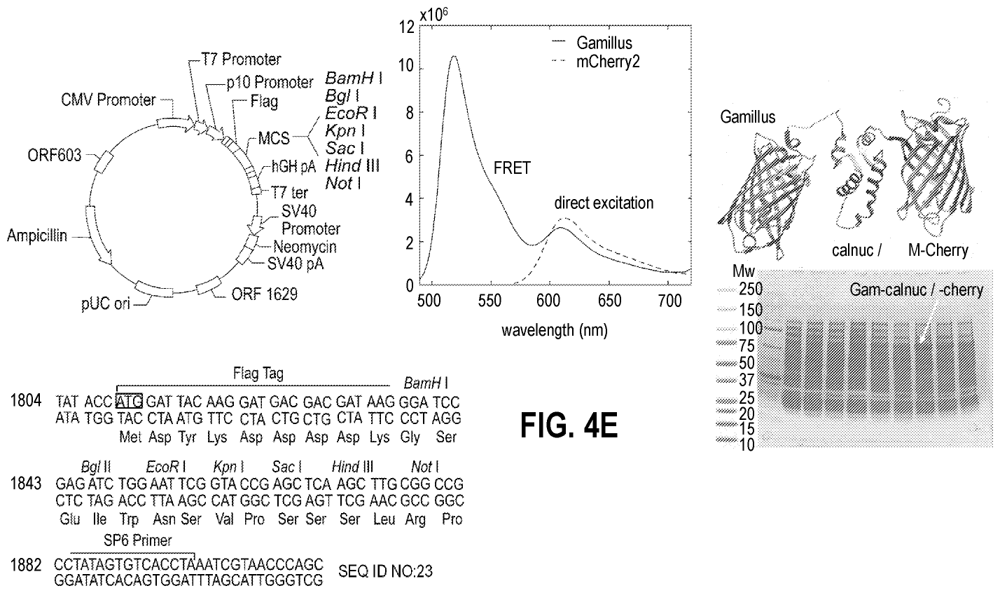

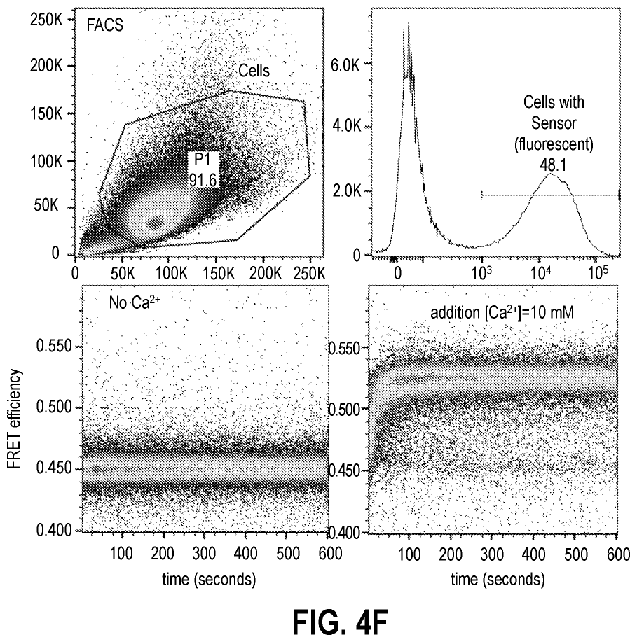

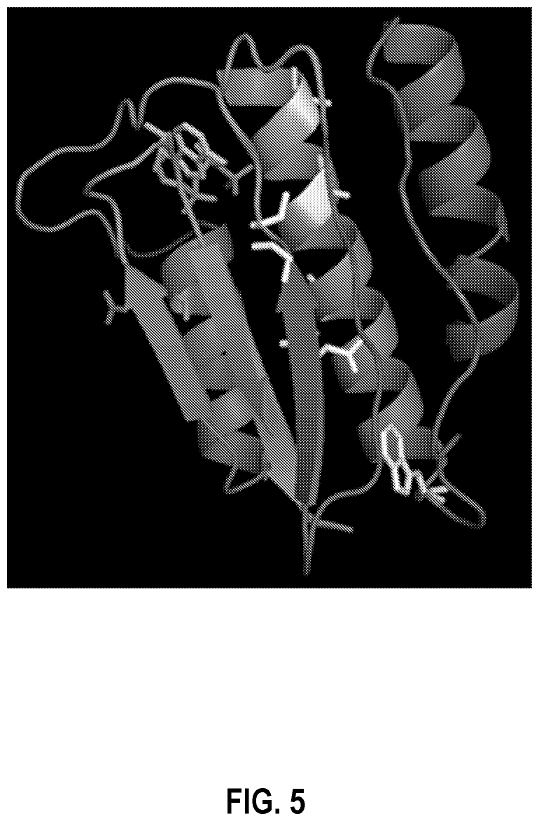

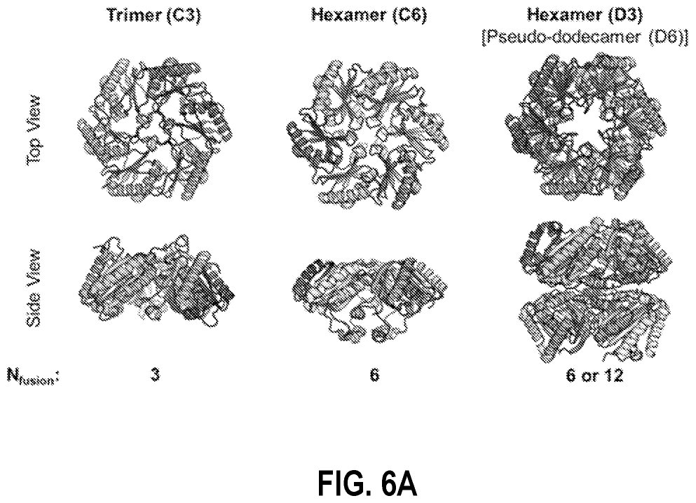

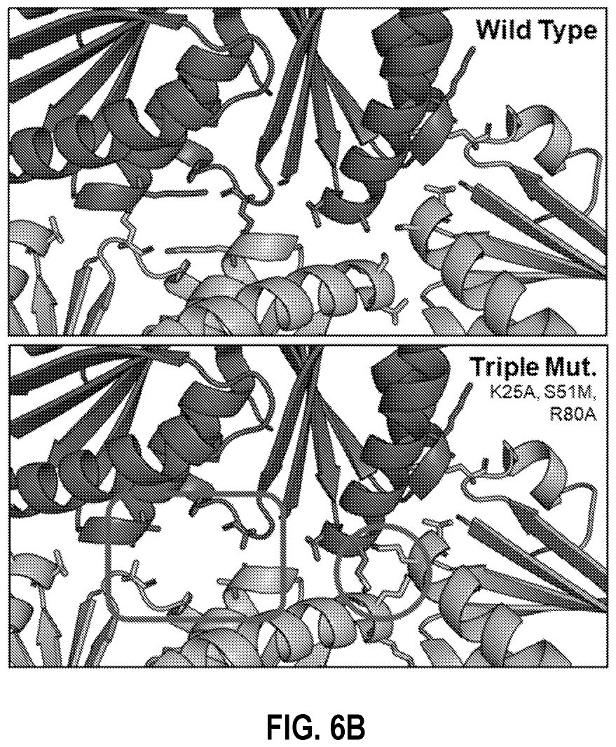

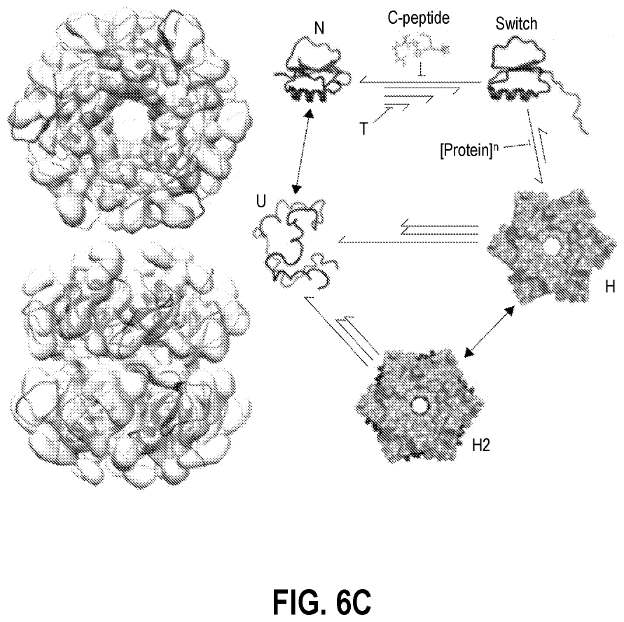

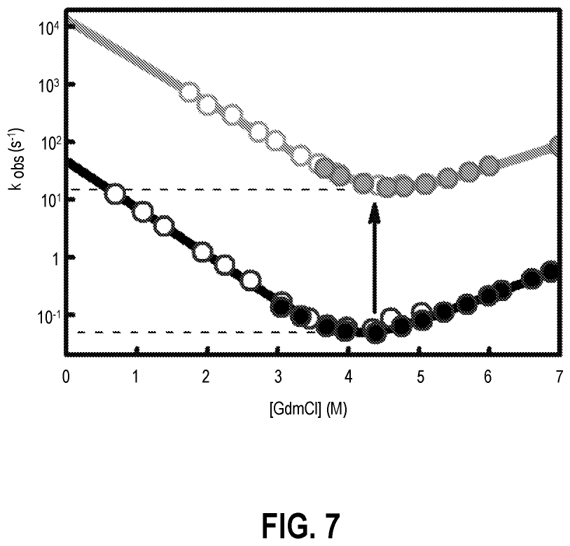

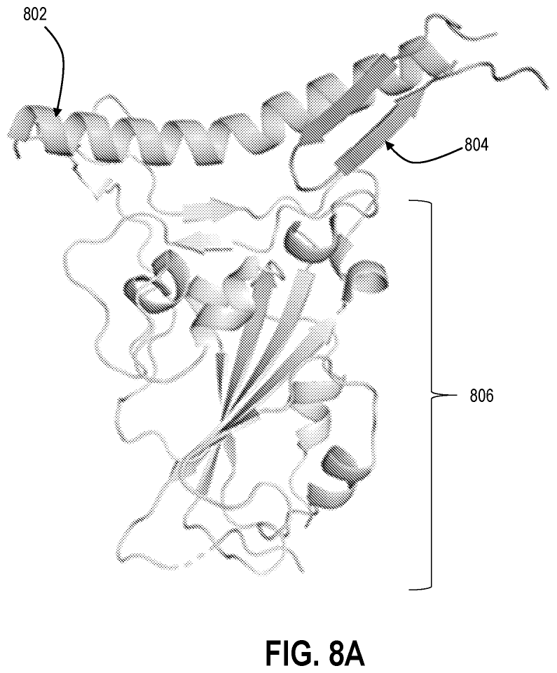

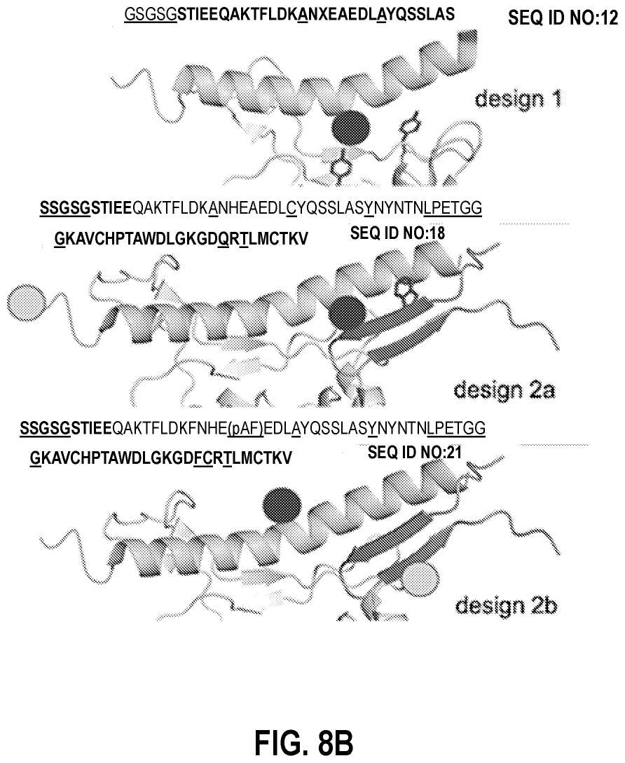

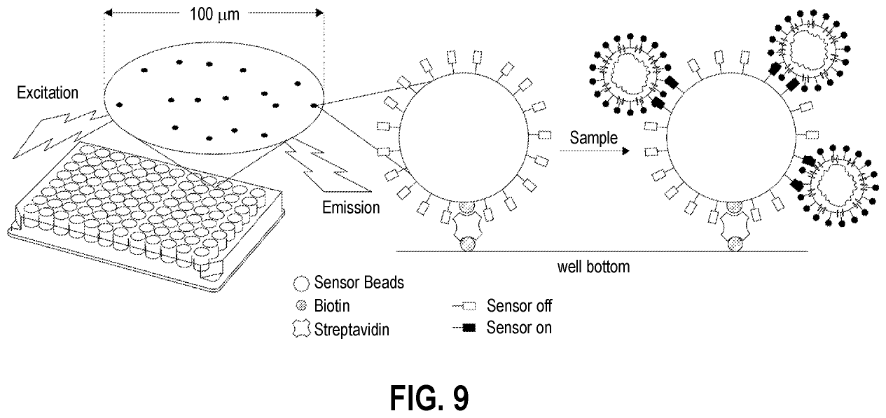

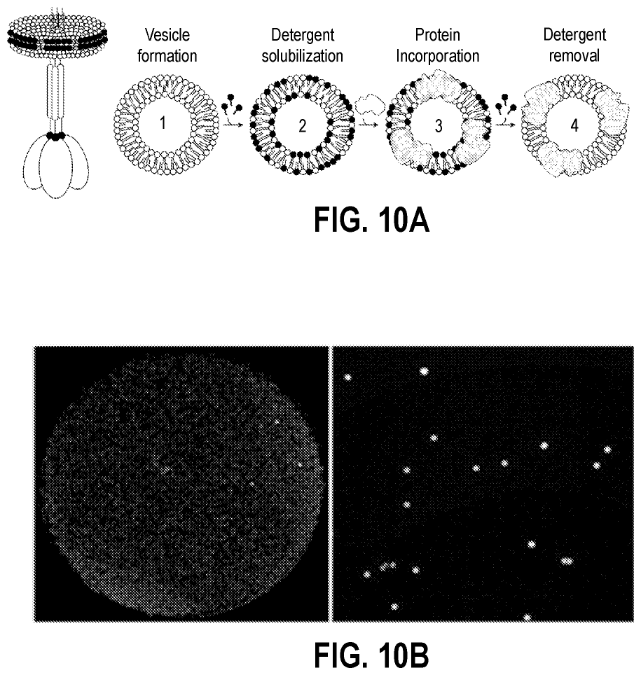

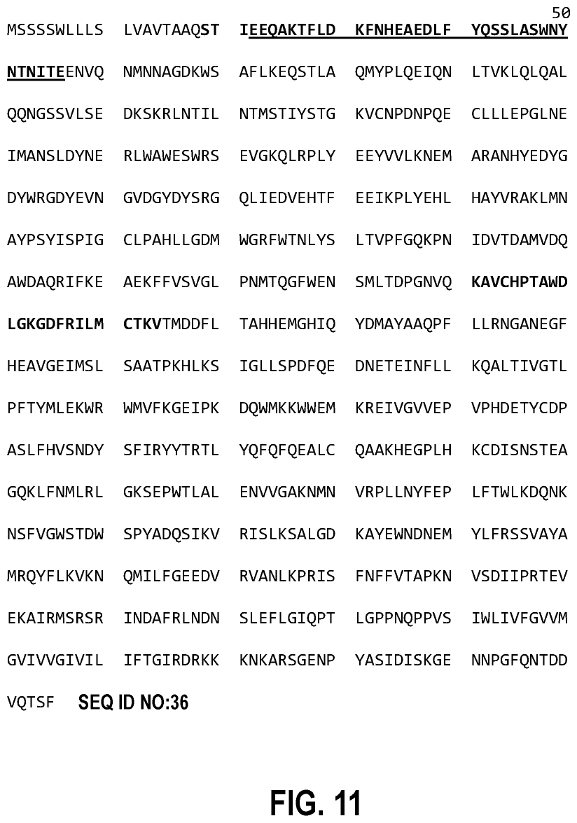

In this case, the liposome-encapsulated B-Hel-B biosensor molecules at various pH values was produced and their fluorescence properties was characterized using a TIRF microscope. A total of 120 images were collected in series (13.407 Hz) with 50 ms exposure time to get a continuous stream of data corresponding to 6 μs of continuous pH monitoring from individual molecules. To identify single molecules, the 512×512 pixel image was separated onto the donor and acceptor halves, and the two images were overlaid. A count threshold of 5,000 was applied to remove the background. Locations of single-molecules were identified from the pixels with maximum intensity, and areas of 5×5 pixels surrounding the maximum intensity pixel were used to encompass the complete image from each molecule (optical resolution with our 1.39 N.A. objective and light in the green-red range is about 200 nm). Once the molecules were identified, the FRET efficiency per frame (50 ms resolution) was calculated by integrating the intensities from the donor and acceptor image slices. FIG. 4.8 shows examples of one molecule pH recordings for pH 4.0, 5.0 and 6.0 (range corresponding to the maximal sensitivity of the B-Hel-B sensor). The images (shown at 50 ms intervals for 1 second) show that at pH 4.0 the example molecule exhibits significantly higher emission in the red (high E) whereas at pH 6.0 it is close to 50-50. The results at pH 5.0 are in between. Calculation of the FRET efficiency (E) by integrating the intensity of the two channels over the 5×5 pixel area shows the change in E as a function of time from each molecule over a period of 6 seconds. The mean E values from these trajectories shows a change of about 10% between pH 6.0 and 4.0. These values are in close agreement with the free diffusion confocal sm-FRET data (FIG. 2G), and are also consistent with the calibration of the sensor in bulk (FIG. 2E note here that the bulk data overestimates the overall E due to incomplete labeling with both fluorophores in the sample, whereas the donor-only molecules are easily identified and discarded in the single-molecule experiments). The data shown in FIG. 21 represents a first direct demonstration of real-time analog recording of pH from a single-molecule biosensor with a 50 ms time response.

These results demonstrate that B-Hel-B can be used as an ultrafast pH biosensor capable of measuring pH with <1 ms response times. Finally, we could also demonstrate the real-time analog monitoring of pH from individual B-Hel-B molecules encapsulated in liposomes.

### Example 2: Integrating a Fluorescence Signal Reporter with a Conformational Rheostat of a pH Biosensor

Downhill (Un)folding coupled to binding is a powerful mechanism for engineering conformational rheostat transducers that convert the ligand-binding event into an analog signal with an extended detecting range. In one example, a pH transducer may be engineered by coupling the histidine (un)protonation with a downhill folding protein (gpW) based on the histidine grafting approach, which showed a sensitivity range from 3 to 9. The coupling of binding with downhill (un)folding is described by Nagpal, S., Luong, T. D. N., Sadqi, M., and Munoz, V, in ACS Synth. Biol 9, 2427-2439 (2020) titled “Downhill (Un)Folding Coupled to Binding as a Mechanism for Engineering Broadband Protein Conformational Transducers”, which is incorporated by reference herein in its entirety. Further, a fluorescence signal reporter is integrated into such a pH transducer by exploiting its distance-dependent property. In one example, the fluorescence reporter is based on the solid distance-dependent property of the PET-based quenching mechanism. Specifically, a tryptophan was introduced to the N-ter of gpW-L7H, which can quench the fluorescence of the ATTO655 labeling a cysteine at the C-ter. The PET construct showed a significant gradual change (2 folds) in fluorescence intensity in a broad range (pH 3 to pH 9). Thus, PET-based quenching provides a promising tool to develop the signal outputs for such a pH biosensor.

As discussed above, protein-based biosensors are analytical tools that can monitor different analytes by employing proteins to design a highly specific and selective biomolecular recognition combined with signal transducer mechanisms coming from the conformational change of the protein upon ligand binding to convert the binding event into a detectable signal readout. In one example, a downhill folding protein is used as a scaffold for the biosensors to engineer a signal transducer with broadband sensitivity and real-time response.

An example pH signal transducer is sensitive to the pH change in >6 orders of magnitude (pH 3 to pH 9) by the histidine grafts. For instance, the histidine was introduced into the hydrophobic cores of the downhill folding protein gpW (W protein of bacteriophage lambda) to trigger a gradual (un)folding of the protein upon binding to the ligand (H+). GpW is a downhill folder with 62 residues long that folds into an α+β topology4 and insensitive to the pH in the range 4-95. Previous approaches employed circular dichroism (CD) spectroscopy to monitor the helical contents change of the proteins upon pH titration as gpW has a high helical content in neutral pH.

Although such pH transducer could produce an analog response, the CD is not a suitable technique to build a signal reporter for a complex environment such as in a living cell or blood sample because this technique would work well only in a sample with high protein concentrations and a ligand that is invisible to CD. In order to address the above issues, in one example, a fluorescence-based biosensor may be used for optical sensing due to their high sensitivity and great potential in biomolecule imaging to study the organization and functions of living systems. Among all the advantages, these fluorescence-based biosensors will be of great interest to detect the analytes at the single-molecule level and generate real-time responses.

In various embodiments fluorescence resonance energy transfer (FRET) or photo-induced electron transfer (PET) may be used to study the conformational flexibility of the proteins. However, their sensitivities are entirely different in terms of their measurable distances.

FRET-based biosensors rely on a pair of fluorophores being covalently attached to the protein. The emission of the donor fluorophore overlaps with the excitation of the acceptor fluorophore, causing the change in emission through Forster Resonance Energy Transfer as a function of the distance between the donor and the acceptor (r). The FRET efficiency depends on the 1/r⁶; thus, the pair acts as a numeric ruler to precisely measure the distance variations between two chosen label-attachment points on the protein as its conformation changed upon binding to the analyte. FRET is a ratiometric (two-wavelength) measurement; therefore, it is independent of donor and acceptor concentrations. The sensitivity of the FRET depends on the characteristic of R0 (Forster distance at which the efficiency of transfer is 50%) that is unique for each donor-acceptor probe pairs. For common Alexa FRET pairs currently commercialized, the sensitivity falls into the 2.5 to 7.5 nm range.

The PET technique relies on the fluorescence quenching phenomenon, which occurs when a fluorophore is being quenched due to electron transfer between its excited state and the quencher's ground-state (Tryptophan or Guanine), which leads to the fluorescence reduction of the fluorophore. PET requires both the fluorophore-quencher pair to be at Van der Waals contact, which means the technique is sensitive in the length scale below 1 nm. Also, PET is exponentially dependent on the fluorophore and quencher proximity. Therefore, PET is a promising tool to monitor distance change in the small biomolecule. However, PET reports the fluorescence intensity at a single wavelength which strongly depends on the fluorophore concentration.

As discussed below, PET fluorescence pair's implementation onto the gpW-L7H construct could generate a high sensitivity analog signal readout in the broad dynamic range (pH3 to pH9).

Results

FRET Strategy for Converting the pH Conformational Rheostat Transducer into an Output Signal.

Previous approaches showed that four single histidine grafts (L7H, M18H, F35H, V40H) and two double histidine grafts (M18H-F35H and F35H-V40H) on gpW exhibits a pH-dependent by triggering structural changes with an extension over >6 orders of magnitude in [H+] using CD technique. L7H showed the most robust effect with linear CD signal changes over a wide range of [H+] (10 nm to 100 mM) among these mutants. Accordingly, the fluorescence integration was employed on this variant. Specifically, an extra cysteine was added at the N-terminal (N-ter), e.g., position 1, and the second cysteine was introduced near the C-terminal (C-ter) by replacing the methionine at position 56. These mutations allow us to chemically label the L7H with a FRET-pair to monitor the end-to-end distance of L7H (L7H-FRET). Another FRET-construct was also designed to measure the distance change between the beta-hairpin and the helix-1. Since the beta-hairpin is highly fluctuating, we were expected to observe a more significant distance variation. In this regard, two cysteines were placed on the M18H-F35H variant by adding one cysteine at the N-ter (position 1) and replacing an aspartate (position 29) with cysteine. This specific variant was chosen for several reasons: (1) F35H was located on the beta-hairpin that formed the hydrophobic pocket with the helix-2, (2) the double mutant is also pH-sensitive a wide range (pH 9-2), and (3) the mutant is partially unfolded at neutral pH. Such mutant may show a greater distance than the end-to-end distance at basic pH and eventually become much more significant when the protein unfolds at acidic pH. In both constructs, Alexa-488 maleimide and Alexa-594 maleimide are used as the FRET pair as they have many advantageous features. For example, they exhibit high brightness, high photostability, good quantum yield, which allow detection with great sensitivity. Moreover, they are pH-insensitive in a wide pH range which makes them suitable for our study.

FIG. 3B shows the distance variations of the two FRET construct as a function of the pH. The higher the FRET efficiency, the shorter the distance between the fluorophore pair. In the L7H-FRET construct, the distance at pH 3 is the lowest and slightly increases with pH change. It indicates that a conformational change does occur upon histidine protonation, which is consistent with the CD results. However, such a change is too minuscule (less than 0.5 nm), which is out of the FRET technique's sensitivity range.

Similarly, in M18H-F35H-FRET (FIG. 3C), a marginal fluctuation among the FRET efficiency upon pH titration is observed. It implies that the N-ter and the beta-hairpin distance did not change drastically with the histidine protonation, and the distance variation is less than 0.1 nm. By comparing these two constructs, the distance between the two terminals is shorter than that of the beta-hairpin and N-ter.

The results clearly showed that although the histidine protonation initiated the conformational change, the proteins remain compact, causing a minimal shift in the distance. Therefore, for this construct, instead of FRET, a PET strategy may be employed to build the reporter.

PET Strategy for Converting the pH Conformational Rheostat Transducer into the Output Signal.

As the end-to-end distance changes of the L7H as a function of pH were determined to be less than 1 nm, the PET technique would theoretically be more suitable to detect the changes. Therefore, two L7H constructs were designed to implement the fluorescence signal readouts. Mainly the tryptophan residue and ATTO 655 were employed as the quencher-fluorophore pair. ATTO 655 is a strong fluorescence acceptor with a high quantum yield. First, the arginine residue at position 3 was replaced with a Tryptophan and the glutamine at position 53 was replaced with cysteine to further labeled the protein with ATTO655. With this construct, the pair of quencher-fluorophore were placed right at the first residue of the helix-1 and the endpoint of helix-2. Such construct (L7H-helix) aimed to precisely monitor the distance changes causing by the helix-random coil transition. Second, the L7H-Cter construct was produced by adding the tryptophan at position 1 and replacing the methionine 36 with cysteines. L7H-Cter with the PET pair placing far from the helical structure and into the random coil segments were used to follow the end-to-end distances.

As shown in FIG. 3D, L7H-helix showed the highest fluorescence intensity at pH 3, then the signal was flat from pH 4 to pH 9. The fluorescence decreased by 2-folds between pH3 and pH 9, which was a significant change. The plot revealed that the distance between the tryptophan and the dye was relatively larger at pH 3, indicating a significant conformational change. At pH4, the distance kept decreasing but with a minimally fluctuating from pH4 to pH 9.

The L7H-Cter exhibited a gradual reduction in fluorescence intensity as pH decreases (pH3 to pH9), indicating that the end-to-end distance was shortened as pH increases. Especially, the intensity change was significantly high within 2-folds, which is considering a good signal change. Thus, by introducing the quencher-fluorophore pair at the two terminals of the L7H, a signal readout system may be produced that can process the conformational pH transducing to an optical output using a highly sensitive technique. Moreover, such pH fluorescence-based biosensor shows a broadband analog sensing that extends over 8 orders of magnitude of [H+].

In this example, PET is utilized as the conformational change associated with the protein folding is relatively tiny. Perhaps the gpW protein variants substantially lost their secondary structures by pH, but their tertiary structures were not drastically impacted.

For in-vivo application, such as monitoring the intracellular pH of different compartments simultaneously, suitable dyes-quencher pairs that can efficiently undergo electron transfer in the living cell may be used. In this regard, a few fluorophores derived from green fluorescence proteins or fluoresceine may be employed.

Experimental Methods

Recombinant Protein Expression and Purification

All gpW variants, including three gpW-L7H and the M18H-F35H variants that targeted different fluorescent probes, were cloned as full genes in the bacterial expression vector pBAT4. The M18H-F35H variant was cloned as SUMO-His-Tag fusions to improve protein expression and purification of unstable protein. The same protocols described in Nagpal et al in in ACS Synth. Biol 9, 2427-2439 (2020) were performed to express and purify gpW mutants and to cleave the protein tag of the double mutants2. The proteins were lyophilized and stored at 253 K.

Fluorescent Labeling of Alexa 488, Alexa 594, and ATTO 655

ATTO 655 maleimide was used to label the PET constructs, and a pair of Alexa 488 maleimide and Alexa 594 maleimide was used for FRET constructs. All proteins were labeled by attaching the fluorescent probed to the inserted cysteines residues. Proteins were dissolved in Tris buffer at pH 6.5, 150 mM NaCl, and reducing agent, TCEP (tris(2-carboxyethyl)phosphine) with a ten times higher than the protein concentration. After incubation for 30 minutes, the TCEP was removed by centrifugal filter (3 kDa cutoff). ATTO 655 was added to the protein solution with a 5 to 1 ratio for the PET constructs. The Alexa 488 and Alexa 594 were added to the protein with the following ratio 1.2:0.8:1 (Alexa 488: Alexa 594: protein) for the FRET constructs. The labeling was performed by incubating 2 hours at room temperature or 277 K overnight with constant stirring. Then 10×BME (β-mercaptoethanol) was added to the protein mixtures. The labeled proteins were purified using RP-HPLC with a 0-95% Acetonitrile gradient with 0.1% trifluoroacetic acid (TFA) for elution. All fractions containing labeled proteins were pooled, and aliquot into small vials and kept at 193 K. The labeled proteins were verified by electrospray mass spectrometry.

Fluorescence Spectroscopy

Proteins samples were prepared at different pH by adding the labeled proteins 20 mM citrate buffer for experiments in the 3-6 pH range, phosphate buffer pH 7, and Tris-HCl buffer for the 8-9 pH range. A final of 0.05% Tween 20 was added to all the buffer for the PET constructs. Fluorescence emission spectra were recorded between 630 nm and 740 nm by exciting the ATTO 655 at 620 nm or between 490 nm and 720 nm by exciting the Alexa 488 at 480 nm. The pH titration on L7H-helix was triplicated, and L7H-Cter was replicated five times to calculate the standard deviation due to the concentration dependence of the PET fluorescence technique.

### Example 3: Calcium (Ca²⁺) Sensors

In the last decade, the Ca²⁺ ion sensing has flourished as burgeoning topic of interest due to the diverse role of Ca⁺² ions in biology. Ca²⁺ signals in cells are either (a) amplitude modulated or (b) frequency modulated, which means the time dependence of the process (and the speed of detection) is a key factor. Moreover, Ca²⁺ levels vary drastically in different cell compartments and at different times or situations: intracellular Ca²⁺ concentration is 10⁴ times lower than extracellular (10⁻³ M) concentration, implying that is very important to develop sensors with as broad a dynamic range as possible, which is another characteristic of our protein-based sensor approach. In fact, available, non protein-based Ca²⁺ sensors detect amplitude pulses but are unable to detect pulse timings.

In order to develop a calcium sensor, similar to other sensors, the (un)folding is coupled to binding principle and further, a protein scaffold that exhibits (or is engineered to exhibit) downhill folding characteristics is needed. In one example, the Calnuc double EF-hand domain from Calbindin, a human protein that is unstructured in absence of Ca⁺², but it folds up onto a characteristic double EF-hand domain in presence of Ca⁺² in pM concentrations may be selected. The EF hands are specific Ca2+ binding motifs that are widespread in Biology (FIG. 4A). Calnuc was cloned as a fusion protein with SUMO, which provided very high expression. Further, the Calnuc fusion protein was engineered to include either a FRET or a PET fluorescence readout based on the NMR structure of the holo form (1SNL.pdb, de Alba, E., Tjandra, N. 2004 Biochemistry 43: 10039-10049, which is incorporated by reference herein in its entirety). For introducing the FRET and PET fluorescence readouts selected locations for the dyes (via thiol labeling of cysteines) and quenchers (mutating to tryptophan) aiming to maximize signal gain were identified.

Further, the Calnuc FRET and PET variants were expressed, purified (using the His-tag of the SUMO construct and a Ni²⁺ column, then removal of SUMO by ULP-1 protease, and second Ni²⁺ purification step), and properly labelled with the required fluorophores. Further, the Ca²⁺ sensing properties of both sensors may be characterized via fluorescence in bulk. The bulk fluorescence experiments demonstrated sensitivity to Ca²⁺ concentrations in the micromolar range, mimicking the changes in Calnuc folding (measured by circular dichroism in the unlabeled protein) upon increases in [Ca²⁺. Interestingly, in this system the FRET and PET readouts produce sensors with distinct properties due to the different distance sensitivity of the two approaches. PET produces an intrinsically binary output because PET either takes place (when the quencher (Trp) is at a distance of <1 nm) or does not take place at all at longer distances. Accordingly, the Calnuc-PET sensor exhibits a very sharp response in which Atto655 fluorescence increases by 5-fold between Ca²⁺ concentrations of 20 μM and 80 μM (FIG. 4B). The Calnuc-PET sensor thus appears as ideal to monitor with extremely high sensitivity the rise of Ca²⁺ inside cells (levels increase from 10₋₇ to 10⁻³ M when the plasmatic membrane is depolarized by an electrical pulse) in terms of a binary output or logical gate (on: basal or off: pulsed Ca²⁺). In some examples, a dual FRET-PET readout for two-color detection onto this sensor by inserting a p-acetyl-L-phenylalanine into the loop connecting both EF-hands (FIG. 4A) for labeling with A488.

The sensor operates in two [Ca²⁺ ] ranges: a) from 10 nM to 1 μM, which corresponds exactly with the cytoplasm concentration range from basal to spikes; b) from 10 to 1 mM, which covers the [Ca²⁺ ] in endoplasmic reticulum, Golgi and extracellular environment. Therefore, the calnuc-FRET sensor is ultrabroadband and can be used to monitor calcium levels in any cellular or tissue compartment, thus solving one of the main limitations of current calcium sensors. The kinetic analysis of the process (from the smFRET data) indicates that the folding rate changes linearly in the two regimes that correspond to the binding affinity of each EF hand, and the unfolding rate decreases at the highest concentrations, which together they result on the broadband response. Also, the kinetics show that this calcium sensor has a response time of about 1 ms, which is 50 fold faster than existing protein-based calcium sensors (most notably Cameleon), and agrees with the timescale of calcium spikes, which indicates this is the first protein-based sensor (and potentially fully genetically encoded, see below) that can work at all required conditions and capture the early timescales of calcium signaling.

The time response is key because the Ca²⁺ time rise in neurons upon depolarization and the pulse frequency found in skeletal muscle cells pulse trains is about 1 ms. Therefore, the calcium sensor is potentially the first protein-based sensor with sufficient time resolution to record such early stages of the Ca²⁺ response in cells (currently only Ca²⁺ chelator-based organic sensors are capable of such response).

High-Sensitivity Fluorescence Readouts for In-Vivo/Intracellular Sensing

GFP Readouts (Full Genetically Encoded Protein Biosensors)

To implement high sensitivity fluorescence readouts for in vivo imaging the production of two-color fluorescent protein fusions (as FRET readouts) was considered that can be fully genetically encoded. For this purpose, we developed a shuttle expression vector for cloning any of our sensor proteins in between a green and a red fluorescence proteins. The vector is based on the pDream2.1 plasmid from GenScript (see FIG. 4E).

This 7.2 kbp plasmid has been designed to permit cloning, amplification and protein expression in the three major expression hosts: bacteria, insect cells (baculovirus) and mammalian cells. pDream2.1 includes the CMV promoter for high-level constitutive expression of genes in a variety of mammalian cell lines, the T7 promoter for convenient expression of genes in bacteria, and the P10 baculovirus promoter for high-level expression of genes in baculovirus-infected insect cells. The multiple cloning site, which includes 7 unique restriction enzyme sites that permit cloning in the same open reading frame, is located in between a poly-His-Flag tag sequence upstream and polyadenylation and transcription termination signals downstream. The poly-His-Flag tag permits single column affinity purification and specific detection of the fused protein using specific and sensitive anti-Flag antibodies. The Flag tag sequence also includes the cleavage site by enterokinase (EK) to eliminate the tag after purification. To this commercial plasmid, the gene for the engineered green fluorescence protein Gamillus or NeonGreen was introduced using the first site (BamHl), and the gene for an engineered red fluorescent protein (either mCherry2 or mScarlet) using the last site (Not1). This provides five usable unique restriction sites to clone any of our sensors as a GFP-sensor-RFP fusion protein. The multiple choices for cloning sites allow us to introduce linkers of various lengths between the sensor and the fluorescent proteins.

These FPs were selected for sensors, wherein: 1) green-red pairs experience FRET efficiency with R₀ of approximately 6 nm, which results in the same 4-8 nm distance range; 2) FPs that are engineered to be monomeric, have high quantum yield and brightness, and insensitivity of their emission to pH and other solutes; 3) have high maturation rates for proper expression and purification of the fusion proteins.

The Gamillus-mCherry 2 fusion alone (without inserted biosensor) as expressed in E. coli and purified wand characterized its fluorescence properties, which rendered a FRET efficiency of approximately 0.75 (FIG. 4E, center). This FRET efficiency value provides a benchmark for the maximum FRET efficiency level that may be expected from the sensors when folded. It is well above 0.5, but still in the linear regime for efficiency versus distance (0.15 to 0.85), which provides an excellent baseline for all the FRET sensors (adding the sensor in between will significantly decrease FRET in the absence of analyte by the extension added by the flexible polypeptide chain and will lead to an efficiency close to 0.75 in the presence of the analyte).

The calnuc-based calcium transducer was cloned into the Gamillus-mCherry 2 fusion. The transducer may be purified and its fluorescence signal may be determined as a function of Calcium in vitro, which recapitulates what was observed in the non-genetically encoded version.

In some examples, the sensor was transfected into mammalian cells and a characterization of the response to calcium of these cells via our biosensor using fluorescence activated cell sorting was performed. These experiments are summarized in FIG. 4F.

### Example 4: ATP Sensors

Adenosine triphosphate (ATP) is known as the energy currency of cell. Energy-requiring processes in the cell consume ATPs, whereas the energy-releasing processes in cell produce it. Tracking the concentrations of ATPs in real time with high time resolution and inside living cells is an essential component to understand the vast majority of cellular processes. ATP sensors are, however, difficult to produce. Current ATP sensors are not selective enough to distinguish between ATP and ADP, which is the fundamental need for an ATP sensor (the entire energy balance in cellular processes is defined by the ATP/ADP ratio). Accordingly, in one example, a recombinant protein-based fluorescence sensor for ATP may be generated with the main goals of selectively distinguishing between ATP and ADP, and working over a wide range of ATP concentrations

In one example, the ATP-binding domain of the protein kinase DesK (PDB ID: 3EHG) from Bacillus subtilis may be used as protein scaffold.

In another example, a more conservative approach from the folding viewpoint for determining the stability of the domain by itself (it is excised from a larger protein) and characterizing its ATP binding affinity may be employed. In this example, the entire ATP-binding domain of DesK may be cloned as it appears in the pdb, and engineered for potential FRET and PET readouts.

Nanoscale Assemblies

One fundamental challenge for the application of non-covalent complementary binding (biomolecular recognition) for the capture/detection of biological agents is to achieve the extremely high affinity required to be effective at the loads/concentrations that these agents are typically found in biologically relevant conditions. For example, the diagnostics of COVID-19 involves the detection of SARS-CoV-2 virions in patient samples (e.g. saliva specimens) that only contain about 10³ to 10⁸ viral particles per mL¹⁻³, and hence require the binding event to take place with affinities in the sub-attomolar range (10⁻²¹-10⁻¹⁸ M). Such concentrations are many orders of magnitude smaller than the 10⁻¹ M affinity of the interaction between streptavidin and biotin, which is the tightest protein-ligand complex known to date 4. Monoclonal antibodies, such as those with widespread use in sandwich detection assays (e.g. ELISA), exhibit affinities around 1 pM, at best. The affinity limit becomes even more of an issue for engineered responsive proteins. This is because the (un)folding upon ligand binding of these proteins involves paying the entropic penalty of folding the protein by the binding free energy, which reduces the overall affinity of the system by typically 2-3 orders of magnitude. This challenge is a fundamental reason why responsive proteins—which are highly desirable since they contain the three basic components required for capture/detection in one molecular element: biomolecular recognition (binding to agent), transducer (conformational change), and response (e.g. fluorescence signal readout)—have been used for biosensing in the nM to pM range, but not for diagnostics or capture/detection of pathogens/biomarkers that are present at very low concentrations.

In one example, a method for boosting the affinity and specificity of the biomolecular recognition events associated to responsive proteins is provided. The strategy is based on enhancing the multivalent binding process. Multivalency emerges when there are multiple copies of the binding site/interface in at least one of the binding partners. When both partners present the same number of copies of complementary binding interfaces and they are all occupied simultaneously, the effect is a multiplier of the overall affinity of the complex: K_{Dm}˜(K_{D1})^{m}, where m is the number of binding sites. When only one binding site is occupied at any given time, there is still a summative effect that is equivalent to an increase in the effective concentration, so that K_{Dm}˜K_{D1}/m. In practice, the net multivalency effect is between these two scenarios because, even with only one binding event per complex, the presence of many locally accessible binding sites increases the recapture probability. To engineer multivalency into a given biomolecular recognition event the key is then to engineer the responsive protein to spontaneously form nanoscale assemblies (oligomers) of specific number of copies (valency) and symmetry in configurations that enable multi-binding to the target. For a given one-to-one binding affinity is in principle possible to tune up that of the assembly through the number of monomers and their spatial configuration in reference to that of the binding target.

Similar considerations apply from the viewpoint of detection, particularly for the implementation of extremely miniaturized detection devices (nano-biosensors). For example, the precision in the detection of a ratiometric two-color fluorescence signal from a single emitting particle is inversely proportional to the number of photons collected according to the shot noise formula:

$${σ}_{SN}^{2} = \frac{E ( 1 - E )}{{N}_{T}}$$

where N_{T} is the total number of collected photons per unit of time, E is the fraction of them that has been emitted by the acceptor fluorophore (red shifted color), and σ_{SN} is the standard deviation in E. The engineering of controlled oligomeric biosensor assemblies may multiply the maximum signal of the device (e.g. mx200 photons for an assembly of m monomers), and accordingly increase its signal to noise ratio (precision/accuracy) by \sqrt{m}. The implementation of biosensor oligomeric assemblies will also enhance the resolution of analog (quantitative) readings from single nanodevices.

To engineer specific nanoscale assemblies of responsive proteins two complementary approaches may be used. One approach is to create ultra-stable assemblies via the incorporation of a specific oligomerization domain connected to the responsive protein by a flexible linker. The other is to create allosterically controllable assemblies by fusion of the responsive protein of interest to the CI2 domain variants that has been engineered to form hexamers and dodecamers on cue.

For the ultra-stable nano-assemblies, a suitably engineered version of the shell subunit protein ccmK2 from the bacterial beta-carboxysome may be used. The carboxysome is a large (micron-scale) assembly that has a roughly icosahedral shape with an outer shell between 80 and 150 nm in diameter that is constructed from a few thousand small protein subunits. Out of this, the primary unit is the “BMC domain” and these small (˜10 kD) domains oligomerize to form the hexamers that form the basic units that assemble into the microcompart-ment facets. The BMC domain spontaneously forms hexameric particles (see FIG. 6A) that are extremely stable to the extent that the monomer has not been observed in solution. In addition to the standard hexameric forming BMC domain, there are also pseudohexameric trimers that result from BMC domain duplication. Finally, in some cases, there are pseudohexameric trimers that individually form “double-ring” structures with D3 (pseudo-D6, and pseudo-dodecameric) symmetry (FIG. 6A). All of these particles result on the protein termini being exposed to the surface, and hence accessible for fusing the responsive proteins to them in either configuration.

In one example, a hexameric BMC domain to fuse it to any given responsive protein of interest (biosensor or capture assembly) via a controllable flexible linker to form C6 responsive hexamers. Alternatively, a trimeric BMC domain may be used to make responsive trimers and a “double ring hexamer” to make a responsive hexamer in which the responsive proteins are placed in both relative orientations (up and down). Finally, a “double ring hexamer” may be used to make a responsive dodecamer by fusing one copy of the responsive protein to each terminus of the tandem BMC domain.

The BMC domains do form higher assemblies that give rise to the micron-sized bacterial microcompartments. To eliminate the capacity of the BMC to form such higher order assemblies we have designed three specific mutations aimed to disrupt the interface and result in single oligomers (e.g. hexamers). FIG. 6B shows the interface between three differently colored hexamers in a 2D BMC lattice assembly of ccmK2. The mutations K25A and R80A are designed to abolish key interactions where two hexamers meet along an edge, while the S51M mutation introduces a steric clash at a three-fold symmetry axis. The resulting sole-hexamer-forming ccmK2 sequence that will be fused to the responsive protein elements is:

(SEQ ID NO: 4)
MSIAVGMIETRGFPAVVEAADSMVAAARVTLVGYEKIGSGRVTVIVRGDV
MGVQASVSAGIEAANRVNGGEVLSTHIIAAPHENLEYVLPIRYTEEVEQF
RTYGVPRGGSHMLKEVWEE

where underlined indicates the three mutations to suppress the formation of the supra-assembly, and in bold is one flexible linker for attachment of the responsive proteins by fusion to the C-terminus of the BMC.

For the allosteric (controllable) dynamic nano-assemblies, the CI2 variants that have been engineered to fold switch as oligomerization domain may be used. The original (wild-type) CI2 protein is naturally monomeric and forms a hyper-stable, unique native 3D structure. Using a purposely designed mutational strategy we produced variants that switch between such native structure and an alternate fold that exposes a favorable inter-monomer interaction interface and hence triggers the assembly of the monomeric protein onto toroidal double hexamers. The hexamers have C6 symmetry and the dodecamer is formed by 2 hexamers interacting in an upside-down configuration (FIG. 6C). In both cases the CI2 monomers in the assembly have their N-terminus pointing to the outside, hence providing a linkage point to fuse any responsive protein of interest. The equilibrium between the two folds can be used as an allosteric switch to externally control the formation of the CI2 nano-assembly on demand via competing/cooperating effects such as temperature, monomer concentration, and a designed short peptide. The thermodynamic model for the allosteric control system for assembly is shown in FIG. 6C. Upon further protein engineering to control the fold switch transition, a series of CI2 mutants may be produced with different intrinsic propensities to fold switch form the dodecamer, a single C6 hexamer, or even remaining monomeric. These CI2 engineered variants may be fused to responsive protein of interest (e.g. a protein that binds to the SARS-CoV2 receptor binding domain) to implement protein systems that will form the assembly templated by multivalent interactions with the complementary target surface (e.g. virion), and will also be externally controllable.

Two exemplary applications of the allosteric nano-assemblies of responsive proteins are: 1) A nanoscale detection/diagnostic system in which the responsive protein will also carry a single fluorophore that will show enhanced fluorescence signal upon assembly on the viral surface. The addition of the designed peptide as external negative control of assembly will permit to profile viral strains through their affinity for the host receptor. 2) A nanoscale pathogen capture system in which specific binding of the responsive protein module to the pathogen will trigger assembly onto dodecamers, which will create a complementary surface for binding to a second pathogen particle (the opposed face of the dodecamer) hence resulting on positive cooperativity and the formation of a capture pathogen mesh interlaced by the double interactions with the nanoscale assembly.

A Toolboxfor Engineering Downhill (Un)Folding Transducers

Building a Catalogue of Molecular Scaffolds.

Using the set of simple structural and physical parameters that define fast folding and minimally cooperative unfolding (both trademarks of downhill folding modules) the catalogue of protein domains may be expanded that could be used as molecular scaffolds for sensing applications. Example molecular scaffolds include antiparallel coiled-coils (NT2), antiparallel 3-helix bundles (EnHD and NCBD, helix-loop-helix motifs (BBL and PDD), minimal alpha-helical domain (villin headpiece), minimal antiparallel beta-sheets (WW domains from FBP11, YAP and Nedd4), helix-hairpin-helix domain (gpW), mid-size beta-antiparallel (SS10352 protein), alpha-beta ladder (cell division reactivation factor), alpha-beta antiparallel sheet (GCC-box binding domain), helix-beta core (Xis protein excisionase), alpha-beta parallel motif (Sperm-associated antigen 7), alpha-beta clasp (CopR repressor), long 3-helix bundles (Insulin gene enhancing protein isl-1 and HIV regulatory protein Vpr), and alpha-beta parallel motif (R3H). This list of domains constitutes a rather comprehensive catalogue of basic folds to be used as molecular scaffolds for applications involving conformational rheostats, and thus it is a key addition to the protein engineering toolset.

The catalogue of protein domains that could be used as molecular scaffolds may be further expanded for responsive protein (e.g. sensing) applications. These include BBL and BBL+, the artificial coiled-coils for pH sensing, gpW (and its mutants), the ATP binding domain of the DesK protein kinase (3EHG.pdb), calnuc calcium binding domain (1SNL.pdb), a series of engineered chymotrypsin inhibitor 2 (CI2) variants that fold switch, which may be used as a transducer to develop a new series of responsive proteins based on fold switching coupled to binding rather than the (un)folding upon binding of our current series of sensors.

Further, another protein scaffold for implementing biosensors with electrical readouts based on a minimalistic membrane channel (OmpC) that is monomeric, can be produced recombinantly and embedded onto membranes in vitro, and can also be gated by the protonation/deprotonation of 2 histidine residues. The channel leads to passive transport of ions and small organic molecules (including monosaccharides), and thus introducing a binding site for the analyte of interest into the gating loop can result in blocked current as reporter of the analyte. This system could allow for the first generation of protein-based electrical sensors for in vivo and intracellular monitoring (it could be attached to a microelectrode).

Engineering Folding Scenarios: From Two-State to Downhill Folding.

In one example, a 64-residue, alpha-beta mixed CI2 domain, which is an extreme case of two-state folding with stability and folding barrier that have been maximized during evolution, may be transformed into a downhill folding conformationally responsive module. The overarching objective is to implement and benchmark a general engineering protocol for transforming two-state folding domains into bona-fide downhill folders, and in the process, to ascertain what is the minimal set of sequence changes that induces such transformation. In one example, the folding rate of CI2 may be accelerated from 50 s⁻¹ to approximately 10,000 s⁻¹ and the unfolding rate from 0.002 s⁻¹ to about 0.3 s⁻¹ (FIG. 7). Therefore, the inventors have shown for the first time that it is possible to engineer the folding transition state ensemble of a slow two-state folder, stabilizing it selectively over the native and unfolded states. This result is very important because this methodology may be applied to jointly speed up the folding and unfolding rates of any protein, which will permit us to increase the time response of any responsive protein (e.g. sensor) based on (un)folding coupled to binding, which is one of the current limitations of existing protein-based sensors of this type, such as the Cameleon Ca²¹ sensor.

### Host Decoy Approach for One-Step Fluorescence Diagnostic of Viruses

In various embodiments, methods and systems are provided for fluorescence-based protein and peptide biosensor devices for the rapid quantitative diagnosis of biological targets, such as viruses (e.g., SARS-CoV-2, the causative agent of COVID-19). These diagnostic devices are based on (1) the process of protein-protein recognition that occurs between the viral particle and its host cell receptor protein (e.g., between the SARS-CoV-2 spike protein and the cellular receptor angiotensin converting enzyme 2) for direct detection of functional viral particles; and (2) multivalent adhesion as amplifier of binding affinity and specificity. The binding event is detected from a change in fluorescence using a two-color reporter system.

In certain embodiments for viral detection and diagnosis, the system involves a set of micron-sized beads coated with a biosensor (i.e., a protein or peptide that binds to the target coupled to a fluorescent reporting system) immobilized on the bottom surface of a multi-well plate. In these embodiments, target (e.g., virus) is detected by interrogation of the microarray by fluorescence excitation and direct measurement. In these embodiments, the diagnostic device has the following features:

(1) The target-specific component of the diagnostic comprises an engineered molecular mimic of the first step in the infection process: the binding event between a target molecule, such as a viral surface protein, and its cellular receptor (e.g., between the spike protein of SARS-CoV-2 and the human angiotensin converting enzyme 2 (ACE-2) receptor). For SARS-CoV-2 diagnosis, the engineered mini-protein scaffold recreates the chemical properties and structure of the ACE-2 region that is recognized by the SARS-CoV-2 spike protein. See Yan et al. (2020) Science 367:1444-1448.

(2) molecular recognition (i.e., binding of biosensor to target) is coupled to production of a fluorescence signal in the visible range, providing rapid, in situ detection of target.

(3) The system provides amplification of affinity, sensitivity and specificity via the functionalization (i.e., coating) of micron-sized beads with protein or peptide sensor molecules (sensor beads). Sensor beads mimic host cells in size and receptor density and thus enhance the affinity for viral particles by many orders of magnitude through the phenomenon of multivalent molecular adhesion. This phenomenon occurs when the two interacting surfaces (here viral target and bead) contain large numbers of binding molecules at a spacing that permits the formation of more than one receptor-target molecule interaction at the same time. See, e.g., Mammen et al. (1998) Angewandte Chemie International Edition 37:2754-2794 and Bell (1978) Science 200:618-627.

(4) Specificity of detection is enhanced when detection depends on multivalent adhesion because non-specific interactions are monovalent and are thus not enhanced by the system.

(5) Multivalent adhesion can be engineered via the density of protein functionalization of the beads (i.e., the coverage density of the biosensor), providing customizable sensitivity and specificity for a viral load range of interest (or to multiple ranges on multiplex arrangements) and any other characteristics of the sample.

(6) The sensor beads can be immobilized on a surface in a fashion analogous to a microarray configuration at a suitably sparse coating density. The density of bead coverage on the surface can be customized for the different implementations of detection to further boost specificity and sensitivity.

(7) Detection is achieved by monitoring fluorescence emission from the virus-complexed biosensors coating the beads. Fluorescence can be detected with extremely high sensitivity, to the extent that fluorescence-based systems are capable of detecting emission from a single molecule. Moerner et al. (2003) Rev. Scientific Instruments 74:3597-3619.

(8) Background signal and absorption of the excitation light are typical problems of fluorescence-based diagnostic systems. To minimize these problems, thereby maximizing sensitivity, fluorescence is stimulated and collected from below the surface, with the sample atop the surface.

(9) The device can be implemented on multiple formats customized for different applications and/or throughput levels, including, for example:
a plate-reader fluorometer format using multi-well plates (e.g., 96-, 384- or 1536-well plates) coated with sensor beads;
high-resolution fiber-optic bundle fluorescence imaging on custom microfluidics chips functionalized with sensor beads
a fluorescence microscope (e.g., a plate-reader cell-imaging fluorescence microscope), and
assaying individual sensor beads using flow cytometric methods (i.e., fluorescence-activated cell sorting).

Accordingly, disclosed herein are compositions and methods for rapid, real-time detection of biological targets such as cells and viruses. Compositions comprise a solid support having affixed to it a plurality of “binding moieties;” i.e., molecules (e.g., proteins or peptides) capable of binding to a target molecule (e.g., a viral protein). By selecting a solid support having roughly the size and shape of a cell, and affixing a plurality of binding moieties to the support, such “cell decoys” are capable of multivalent binding, as in naturally-occurring host-pathogen interactions, such as viral infections. In addition, the sensitivity of detection using such cell decoys, can be adjusted, both by the number of binding moieties affixed to the support (i.e., the “coverage density”) and by the number of cell decoys used in an assay.

In the compositions described herein, a binding moiety is fused (e.g., covalently) to a fluorescent moiety (i.e., a biosensor). The fluorescent moiety is one whose fluorescence properties (e.g., intensity, wavelength) are dependent on its environment, such that, for example, its fluorescence emission has a certain value (e.g., of intensity or wavelength) when the biosensor is free (i.e., not bound to a target), but has a different value when the biosensor is bound to a target. That is, one or more of the fluorescence properties of the fluorescent moiety are altered upon binding to the target.

Accordingly, provided herein are compositions for detection of a target, the compositions comprising (a) a binding moiety that binds to a molecule on the surface of the target; and (b) a fluorescent moiety having one or more first emission properties when the binding moiety is not bound to the target, and one or more second emission properties when the binding moiety is bound to the target. Such compositions are alternatively denoted “biosensors,” “sensor peptides,” “engineered responsive proteins,” “sensor proteins.”

In certain embodiments, the binding moiety is a peptide or a protein. However, any molecule that binds to a molecule on the surface of the target (e.g., a lectins, a nucleic acid, etc.) can be used as a binding moiety.

In certain embodiments, the emission properties are, for example, fluorescence intensity and/or fluorescence emission wavelength. In other embodiments, such as energy transfer systems, the emission property is a ratio of emissions at two different wavelengths, and the ratio changes upon binding of the biosensor to the target.

For example, in some embodiments, the first emission property is fluorescence intensity at a particular wavelength, and the second emission property is increased fluorescence intensity at a higher wavelength. This type of change occurs in certain systems that undergo excited state intramolecular proton transfer (ESIPT) using fluorophores such as, for example, 2-(2-furyl)-3-hydroxychromone (FHC). In other ESIPT-based systems, the first emission property is fluorescence intensity at a particular wavelength, and the second emission property is increased fluorescence intensity at a lower wavelength, using fluorophores such as, for example, 6-N,N-dimethylamino-2,3-naphthalimide (6-DMN).

In additional embodiments, the first emission property is fluorescence intensity at a particular wavelength, and the second emission property is decreased fluorescence intensity at that wavelength. This type of change occurs in certain systems that undergo photoinduced electron transfer using oxazine fluorophores such as, for example, Atto 655.

In additional embodiments, fluorescence systems that participate in Forster resonance energy transfer (FRET) are used. In these systems, the first emission property is a ratio of fluorescence emission intensity at two different wavelengths, and the second emission property is an increase in emission intensity at one of the two wavelengths, and a decrease in emission intensity at the other wavelength; i.e., a change in the ratio of emission intensities. Many fluorophore pairs that participate in FRET are known; one example being the combination of Alexa488 and Alexa594.

In certain embodiments, the target is a cell. The cell can be a prokaryotic cell (e.g., a pathogenic bacterium) or a eukaryotic cell (e.g., a pathogenic fungus). Additional cellular targets can be animal cells (e.g., human cells) such as, for example, tumor cells.

In certain embodiments, the target is a virus. Pathogenic human viruses are known and are enumerated elsewhere herein. In some embodiments, the binding moiety binds to a viral surface protein.

In certain embodiments, the target is a human virus and the virus is SARS-CoV-2, the causative agent of COVID-19 disease. In certain of these embodiments, the binding moiety binds to a SARS-CoV-2 surface protein. In some embodiments the SARS-CoV2 surface protein is the SARS-CoV-2 spike (S) protein. In some embodiments, the binding moiety binds to the receptor binding domain of the SARS-CoV-2 spike protein. In certain of these embodiments, the binding moiety comprises a portion of angiotensin converting enzyme-2 (ACE2). In certain of these embodiments, the binding moiety is a peptide comprising a portion of the peptidase domain of angiotensin converting enzyme-2 (ACE2). Portions of the ACE2 peptidase domain for design of a binding moieties include the following amino acid sequences or portions thereof:

(SEQ ID NO: 5)
STIEEQAKTFLDKFNHEAEDLFYQSSLASWNYNTN
(α-helix)
(SEQ ID NO: 6)
KAVCHPTAWDLGKGDFRILMCTKV
(β-hairpin).
(SEQ ID NO: 7)
GSGSGSTIEEQAKTFLDKANCEAEDLAYQSSLASWNYNTN;
where a fluorophore can be at position 21 between
N and E.
(SEQ ID NO: 8)
SSGSGSTIEEQAKTFLDKANHEAEDLCYQSSLASYNYNTNLPETGGGK
AVCHPTA
(SEQ ID NO: 9)
WDLGKGDQRTLMCTKV;
(SEQ ID NO: 10)
SSGSGSTIEEQAKTFLDKFNHECEDLAYQSSLASYNYNTNLPETGGGK
AVCHPTA;
(SEQ ID NO: 11)
WDLGKGD(pAF)RTLMCTKV;

(pAF) Represents Para-Acetyl-Phenylalanine.

Also provided are “sensor beads” comprising a solid support that comprises plurality of biosensors. In certain embodiments, the solid support is a sphere or a spheroid. The solid support can have a diameter (for a sphere) or a nominal diameter (for a spheroid) of 0.5 μm or greater. In certain embodiments, the solid support is a polystyrene bead; e.g., a polystyrene spheroid with a diameter of approximately 1p m and said plurality is between 500 and 15,000.

The number of biosensors that are present on a solid support can be determined, to provide maximal sensitivity and specificity, based on the size of the target, the density of the receptor for the target on the host cell, the affinity of the target for its receptor and the size of the support. In certain embodiments, the number of biosensors present on a solid support is between 500 and 15,000. The number of biosensors present on a sensor bead, combined with the size of the bead, can be adjusted so that the sensor bead recreates a process of multivalent adhesion of binding moiety to target, similar to the process that occurs during binding of the target (e.g., virus) to its host cell. To this end, the size of the bead can be chosen to approximate the size of the target cell, and the density of biosensors on the bead can be chosen to be similar to the density of the receptor for the target on the host cell.

For a sensor bead that is used to detect a virus that binds by way of a viral surface protein to a receptor on the surface of a host cell; the density of the biosensors on the solid support can be determined by the following criteria: (a) the density of the receptor on the surface of the host cell, (b) the size of the virus, (c) the affinity of the viral surface protein for the receptor; and (d) the size of the solid support. In certain embodiments of a sensor bead, the virus is SARS-CoV-2, the viral surface protein is the spike protein (S protein); and the receptor is angiotensin-converting enzyme-2 (ACE2).

Also provided are a number of detection systems comprising the sensor beads described above. Thus, in certain embodiments, provided herein is a detection system comprising (a) a surface that is transparent to light, (b) a plurality of sensor beads, (c) a light source and (d) apparatus for measuring fluorescence emission images. In certain embodiments, the surface that is transparent to light is the bottom of a well of a multiwell plate (e.g., a microtiter plate). In certain embodiments, sensor beads are affixed to the light-transparent surface; e.g., through avidin-biotin or streptavidin-biotin interactions. In additional embodiments, illumination (and optionally fluorescence detection) occurs from below the light-transparent surface. Use of multiwell plates allows a plurality of assays to be conducted simultaneously; for example, sensor beads specific for a particular target are placed in the wells, and a different sample is added to each well for assay. In addition, certain wells can contain known amounts of target, to generate a standard curve.

In certain embodiments, provided herein is a system for detection of the SARS-CoV-2 coronavirus, comprising (a) a surface that is transparent to light, (b) a plurality of sensor beads, (c) a light source and (d) apparatus for measuring fluorescence emission images; wherein the sensor beads comprise a binding moiety selected from one or more of the following amino acid sequences:

(SEQ ID NO: 12)
GSGSGSTIEEQAKTFLDKANCEAEDLAYQSSLAS;
where a fluorophore can be at position 21 between
N and E.
(SEQ ID NO: 8)
SSGSGSTIEEQAKTFLDKANHEAEDLCYQSSLASYNYNTNLPETGGGK
AVCHPTA;
(SEQ ID NO: 9)
WDLGKGDQRTLMCTKV;
(SEQ ID NO: 13)
SSGSGSTIEEQAKTFLDKFNHE(pAF)EDLAYQSSLASYNYNTNLPETGG
GKAVCHPTA;
(SEQ ID NO: 14)
WDLGKGDFCRTLMCTKV;
(SEQ ID NO: 7)
GSGSGSTIEEQAKTFLDKANCEAEDLAYQSSLASWNYNTN;
where a fluorophore can be at position 21 between
N and E.
(SEQ ID NO: 8)
SSGSGSTIEEQAKTFLDKANHEAEDLCYQSSLASYNYNTNLPETGGGK
AVCHPTA;
(SEQ ID NO: 10)
SSGSGSTIEEQAKTFLDKFNHECEDLAYQSSLASYNYNTNLPETGGGK
AVCHPTA;
and
(SEQ ID NO: 11)
WDLGKGD(pAF)RTLMCTKV;

Wherein (pAF) represents para-acetyl-phenylalanine. In additional embodiments of this system for detection of SARS-CoV-2, the density of the sensor beads per unit area of the light-transparent surface is between one sensor bead per 320 μm² and one sensor bead per 32 mm².

In certain embodiments, the wells of a microwell plate are used as the light-transparent surfaces, and analysis of fluorescence is conducted using a plate-reader fluorescence imager.

Also provided are detection systems capable of analyzing individual sensor beads using bundled fiber optic cables. Accordingly, in certain embodiments, provided herein is a detection system comprising a fiber optic bundle and a plurality of sensor beads. Also provided are detection systems capable of analyzing individual sensor beads using fluorescence-activated cell sorters. Accordingly, in certain embodiments, provided herein is a detection system comprising a fluorescence-activated cell sorter and a plurality of sensor beads.

In certain embodiments, provided herein is a two-dimensional matrix comprising a plurality of sensor beads, wherein (a) in a first dimension, the size of the sensor bead varies, and (b) in a second dimension, the density of the biosensor, per unit area of the sensor bead, varies. Such matrices can be used to assay a wide range of target concentrations, such that certain points on the matrix are sensitive enough to detect a very low level of target; while other points on the matrix are able to detect a very high level of target without saturation of the binding moieties.

In certain embodiments, each point on the matrix comprises a plurality of sensor beads; for example, each point on the matrix is the well of a microwell plate, wherein each well comprises a plurality of sensor beads.

Also provided are methods for the detection of a biological target (e.g., a virus; e.g., SARS-CoV-2) in a sample using the compositions, binding moieties, fluorescent moieties, biosensors, sensor beads and detection systems described above. Thus, in certain embodiments, provided herein are methods for detecting the presence of a target in a sample, the methods comprising (a) measuring a first emission property of a sensor bead, (b) contacting one or more of the sensor beads with a sample to form an assay mixture, and (c) measuring a second emission property of the sensor bead; wherein a difference between the first emission property and the second emission property indicates the presence of the target in the sample. In certain embodiments, the first and second emission properties are the result of irradiation of the assay mixture. The first and second emission properties can be one or more of fluorescence intensity, emission wavelength or the ratio of emission intensities at two different wavelengths.

Samples can be, for example, saliva, sputum, a nasopharyngeal swab or an oral swab.

Contacting and measurement of emission properties can occur, for example, in a chamber, such as, for example, the well of a microtiter plate. The sensor beads can be affixed to the plate, as described above, and sensor beads can be present at densities described elsewhere herein. In certain embodiments, irradiation of the assay mixture, and measurement of emission properties, are conducted from below the well.

Measurement of emission properties can also be conducted using a fiber optic bundle or a fluorescence-activated cell sorter. Thus, in certain of the methods disclosed herein, contacting sensor beads with a sample, and measurement of first and second emission properties, occurs in a fluorescence-activated cell sorter.

### Nucleic Acid and Amino Acid Sequence Homology

This disclosure provides amino acid sequences that are used as binding domains for detection of targets, such as biological targets, such as viruses. It is to be understood that this disclosure also provides conservative amino acid substitutions in the sequences disclosed herein. The term “conservative amino acid substitution” refers to grouping of amino acids on the basis of certain common structures and/or properties. With respect to common structures, amino acids can be grouped into those with non-polar side chains (glycine, alanine, valine, leucine, isoleucine, methionine, proline, phenylalanine and tryptophan), those with uncharged polar side chains (serine, threonine, asparagine, glutamine, tyrosine and cysteine) and those with charged polar side chains (lysine, arginine, aspartic acid, glutamic acid and histidine). Amino acids containing aromatic side chains include phenylalanine, tryptophan and tyrosine. Heterocyclic side chains are present in proline, tryptophan and histidine. Within the group of amino acids containing non-polar side chains, those with short hydrocarbon side chains (glycine, alanine, valine, leucine, isoleucine) can be distinguished from those with longer, non-hydrocarbon side chains (methionine, proline, phenylalanine, tryptophan). Within the group of amino acids with charged polar side chains, the acidic amino acids (aspartic acid, glutamic acid) can be distinguished from those with basic side chains (lysine, arginine and histidine).

A functional method for defining common properties of individual amino acids is to analyze the normalized frequencies of amino acid changes between corresponding proteins of homologous organisms (Schulz, G. E. and R. H. Schirmer, Principles of Protein Structure, Springer-Verlag, 1979). According to such analyses, groups of amino acids can be defined in which amino acids within a group are preferentially substituted for one another in homologous proteins, and therefore have similar impact on overall protein structure (Schulz & Schirmer, supra). According to this type of analysis, the following groups of amino acids can be conservatively substituted for one another:
(i) amino acids containing a charged group, such as Glu, Asp, Lys, Arg and His,
(ii) amino acids containing a positively-charged group, such as Lys, Arg and His,
(iii) amino acids containing a negatively-charged group, such as Glu and Asp,
(iv) amino acids containing an aromatic group, such as Phe, Tyr and Trp,
(v) amino acids containing a nitrogen ring group, such as His and Trp,
(vi) amino acids containing a large aliphatic non-polar group, such as Val, Leu and Ile,
(vii) amino acids containing a slightly-polar group, such as Met and Cys,
(viii) amino acids containing a small-residue group, such as Ser, Thr, Asp, Asn, Gly, Ala, Glu, Gln and Pro,
(ix) amino acids containing an aliphatic group, such as Val, Leu, Ile, Met and Cys, and
(x) amino acids containing a hydroxyl group, such as Ser and Thr.

Thus, as exemplified above, conservative substitutions of amino acids are known to those of skill in this art and can be made generally without altering the biological activity of the resulting molecule. Those of skill in this art also recognize that, in general, single amino acid substitutions in non-essential regions of a polypeptide do not substantially alter biological activity. See, e.g., Watson, et al., “Molecular Biology of the Gene,” 4th Edition, 1987, The Benjamin/Cummings Pub. Co., Menlo Park, CA, p. 224.

It is also to be understood that the amino acid sequences provided herein can comprise one or more non-naturally-occurring amino acid(s). A “non-naturally occurring amino acid” refers to an amino acid that is not one of the 20 common amino acids found in naturally-occurring proteins. Other terms that may be used synonymously with the term “non-naturally occurring amino acid” are “non-natural amino acid,” “unnatural amino acid,” “non-naturally-encoded amino acid,” and variously hyphenated and non-hyphenated versions thereof. The term “non-naturally occurring amino acid” also includes, but is not limited to, amino acids that occur by modification (e.g., post-translational modifications) of a naturally occurring amino acid (including but not limited to, the 20 common amino acids, pyrolysine or selenocysteine). Examples of this type of non-naturally-occurring amino acid include, but are not limited to, N-acetylglucosaminyl-L-serine, N-acetylglucosaminyl-L-threonine, and O-phosphotyrosine.

Also provided herein are amino acid sequences that are substantially identical to those disclosed herein, and amino acid sequences that have a greater than 50% identity with the amino acid sequences disclosed herein. The terms “identical” or percent “identity,” in the context of two or more nucleic acids or polypeptides, refer to two or more sequences or subsequences that are the same or that have a specified percentage of nucleotides or amino acid residues that are the same, when compared and aligned for maximum correspondence, as measured using a sequence comparison algorithm such as those described below for example, or by visual inspection. Accordingly, amino acid sequences that have greater than 55%, greater than 60%, greater than 70%, greater than 75%, greater than 80%, greater than 85%, greater than 90%, greater than 95%, greater than 96%, greater than 97%, greater than 98%, greater than 99%, greater than 99.5%, and greater than 99.9% identity with the sequences disclosed herein are also provided.

The phrase “substantially identical,” in the context of two nucleic acids or polypeptides, refers to two or more sequences or subsequences that have at least 75%, at least 85%, at least 90%, at least 95%, or at least 99% nucleotide or amino acid residue identity, when compared and aligned for maximum correspondence, as measured using a sequence comparison algorithm such as those described below for example, or by visual inspection. Substantial identity can exist over a region of the sequences that is at least about 10, about 20, about 30, about 40, about 50 or about 60 residues in length (or any integral value therebetween).

“Sequence similarity” refers to the percent similarity in nucleotide or amino acid sequence (as determined by any suitable method) between two or more polynucleotide or polypeptide sequences. Two or more sequences can be anywhere from 0-100% similar, or any integral value therebetween. Furthermore, sequences are considered to exhibit “sequence identity” when they are at least about 80-85%, at least about 85-90%, at least about 90 92%, at least about 93 95%, at least about 96 98%, or at least about 98-100% identical to each other (including all integral values falling within these described ranges). Additionally, one of skill in the art can readily determine the proper search parameters to use for any given sequence in the programs described herein. For example, the search parameters may vary based on the size of the sequence in question.

Techniques for determining nucleic acid and amino acid sequence similarity are known in the art. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482 489 (1981). This algorithm can be applied to amino acid sequences by using the scoring matrix developed by Dayhoff, Atlas of Protein Sequences and Structure, M. O. Dayhoff ed., 5 suppl. 3:353 358, National Biomedical Research Foundation, Washington, D.C., USA, and normalized by Gribskov (1986) Nucl. Acids Res. 14(6):6745 6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). An additional method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

For sequence comparison, typically one sequence acts as a reference sequence, to which one or more test sequence(s) are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection. See generally, Current Protocols in Molecular Biology, (Ausubel, F. M. et al., eds.) John Wiley & Sons, Inc., New York (1987-1999, including periodic supplements). Sequence comparisons using these programs are typically conducted using the default parameters specific for each program, but custom parameters can also be used, depending on the nature and/or purpose of the comparison.

Another example of an algorithm that is suitable for determining percent sequence identity and sequence similarity is the BLAST algorithm, which is described in Altschul et al. (1990) J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. This is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: (1) the cumulative alignment score falls off by the quantity X from its maximum achieved value; (2) the cumulative score goes to zero or below, due to the accumulation of one or more negative-scoring residue alignments; or (3) the end of either sequence is reached.

The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. See Henikoff & Henikoff (1992) Proc. Natl. Acad. Sci. USA 89:10915-10919. In addition to calculating percent sequence identity, the BLAST algorithm also performs a statistical analysis of the similarity between two sequences (see, e.g., Karlin & Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### Targets

The methods and compositions disclosed herein are used for the detection of targets (e.g., biological targets). Biological targets include, for example, cells and viruses. In certain embodiments, the invention is used for the rapid detection of pathogens.

Accordingly, in certain embodiments, the methods and compositions disclosed herein are used to detect pathogenic microorganisms, e.g., pathogenic bacteria. Examples of pathogenic bacteria include Escherichia (e.g., E. coli.), Salmonella (e.g., S. typhimurium, S. enterica, S. typhi, S. paratyphi), Shigella (e.g., S. dysenteriae), Staphylococcus (e.g., S. aureus, MRSA, S. haemolyticus, S. epidermidis), Streptococcus (e.g., S. pneumoniae, S. pyogenes), Enterococcus (e.g., E. faecalis), Clostridium (e.g., C. difficile, C. perfringens, C. tetani), Yersinia (e.g., Y. pestis), Campylobacter (e.g., C. jejuni), Pseudomonas (e.g., P. aeruginosa), and Mycobacterium (e.g., M. tuberculosis).

Surface molecules of pathogenic bacteria (including the examples of pathogenic bacteria listed above) such as surface proteins, surface glycoproteins, surface lipids or surface sugars (i.e., target molecules) are known and/or can be identified using methods available to the ordinarily skilled artisan. Furthermore, molecules (such as proteins or peptides) that bind to bacterial surface proteins can be identified using, for example, methods such as the two-hybrid system, co-immunoprecipitation assays, protein fragment complementation assays (PCAs), enzymatic complementation assays, fluorescence resonance energy transfer (FRET), biomolecular fluorescence complementation (BiFC), bioluminescence resonance energy transfer (BRET), tandem affinity purification (TAP), gel retardation, affinity chromatography, mass spectroscopy, proximity ligation assays, phage display, isothermal titration calorimetry, surface plasmon resonance, microscale thermophoresis and chemical crosslinking assays. Other methods known in the art for identifying binding between two molecules can also be used.

In additional embodiments, the methods and compositions disclosed herein are used for the identification of pathogenic viruses. Both human (i.e., clinical) and veterinary viruses can be identified.

Exemplary pathogenic human viruses that can be identified using the methods and compositions disclosed herein include polioviruses, influenza viruses, rhinoviruses, coronaviruses (e.g., SARS-CoV, SARS-CoV-2), rabies virus, retroviruses, lentiviruses (e.g., HIV), hepatitis viruses, rhabdoviruses, poxviruses (e.g., smallpox, chicken pox), Rubella virus, measles virus, herpesviruses, adenoviruses, caliciviruses, respiratory syncitial virus, arboviruses and the like.

Exemplary veterinary viruses that can be identified using the methods and compositions disclosed herein include canine parvovirus, canine distemper virus, feline leukemia virus, veterinary herpesviruses and arboviruses.

Surface molecules of pathogenic viruses (including the examples of pathogenic viruses listed above) such as surface proteins, surface glycoproteins, surface lipids or surface sugars (i.e., target molecules) are known and/or can be identified using methods available to the ordinarily skilled artisan. Furthermore, molecules (such as proteins or peptides) that bind to viral surface proteins can be identified using the methods described above for identification of bacterial surface proteins; i.e., two-hybrid systems, co-immunoprecipitation assays, protein fragment complementation assays (PCAs), enzymatic complementation assays, fluorescence resonance energy transfer (FRET), biomolecular fluorescence complementation (BiFC), bioluminescence resonance energy transfer (BRET), tandem affinity purification (TAP), gel retardation, affinity chromatography, mass spectroscopy, proximity ligation assays, phage display, isothermal titration calorimetry, surface plasmon resonance, microscale thermophoresis and chemical crosslinking assays. Other methods known in the art for identifying binding between two molecules can also be used.

In certain embodiments, the target is the coronavirus SARS-CoV-2, the causative agent of COVID-19 disease.

In further embodiments, eukaryotic cells can be detected using the methods and compositions disclosed herein. Cells that present a characteristic abundance and/or distribution of a distinctive target molecule (e.g., a protein such as, for example, a tumor antigen) on their surface (such as, for example, tumor cells, e.g., circulating tumor cells) can be detected. Methods for identifying binding moieties that bind to a surface molecule of a eukaryotic cell are the same as those described above for identifying binding moieties that bind to bacteria and viruses.

### Binding Moiety

The compositions described herein comprise a binding moiety, which is a molecule that is able to bind specifically to a target molecule. A binding moiety can be any biological or synthetic molecule that is capable of specific binding to a molecule on the surface of the target (e.g., a bacterial or viral surface protein). Exemplary binding moieties include proteins, peptides, protein or peptide analogues, aptamers, lectins, sugars, nucleic acids and nucleic acid analogues.

For binding moieties that are proteins, it is often possible to identify a region of the protein that is responsible for its binding to the target molecule. In such cases, a short peptide encompassing this binding region can be used as a binding moiety. Methods for peptide synthesis are known in the art and include, for example, solid-phase peptide synthesis, or coupled transcription and translation of naturally-occurring or synthetic DNA sequences, either in vivo or in vitro.

As elaborated elsewhere in this disclosure, sensor beads are designed to contain a plurality of binding moieties, thereby making the sensor beads capable of multivalent adhesion to a target. This, plus the fact that the size and shape of the sensor bead resembles those of a cell, enhances affinity and specificity of binding by many orders of magnitude, compared to natural binding affinities. Thus, even viruses that bind to their target weakly, such as influenza virus (which binds to target cells with millimolar affinity), can be detected with high sensitivity.

In certain embodiments, the target is a cell and the target molecule is a cell-surface protein. In certain embodiments, the cell is a eukaryotic cell (e.g., a tumor cell) and the binding moiety binds to a protein on the surface of the cell (e.g., a tumor antigen).

In additional embodiments, the cell is a prokaryotic cell and, in further embodiments, the cell is a bacterium. Accordingly, in some embodiments, the binding moiety is a molecule (e.g., a protein or peptide) that binds to a bacterial cell-surface protein.

In certain embodiments, the target is a virus and the target molecule is a viral surface protein. Accordingly, in some embodiments, the binding moiety is a molecule (e.g., a protein or peptide) that binds to a viral surface protein. To provide but one example, certain embodiments provide methods for detection of the SARS-CoV-2 virus, the causative agent of COVID-19, using a binding moiety that binds to a surface protein of the SARS-CoV-2 virus known as the spike protein (S-protein). It is known that the SARS-CoV-2 virus uses the cell surface receptor angiotensin converting enzyme 2 (ACE2) to gain entry to the host cell. Accordingly, in certain embodiments human ACE2, or a portion thereof, is used as a binding moiety. The amino acid sequence of human ACE2 is shown in FIG. 11.

The region of ACE2 to which SARS-CoV-2 spike protein binds is located in the peptidase domain (PD) of ACE2. Accordingly, in additional embodiments, the peptidase domain of ACE2 (whose amino acid sequence is known in the art) is used as a binding moiety. More refined definition of the regions within the peptidase domain that bind to the SARS-CoV-2 spike protein revealed that two short peptide sequences—an α-helix and a β-hairpin—within the peptidase domain comprise the regions of ACE2 that are bound by the SARS-CoV-2 spike protein.

The amino acid sequence of α-helix present in the peptidase domain of the ACE2 receptor is:

(SEQ ID NO: 15)
STIEEQAKTFLDKFNHEAEDLFYQSSLASWNYNTNITE
or
(SEQ ID NO: 5)
STIEEQAKTFLDKFNHEAEDLFYQSSLASWNYNTN

Accordingly, in certain embodiments, the foregoing α-helix amino acid sequence, or a derivative, or a portion thereof, is used as a binding moiety, or as part of a binding moiety, for detection of SARS-CoV-2.

The amino acid sequence of the β-hairpin present in the peptidase domain of the ACE2 receptor is:

(SEQ ID NO: 6)
KAVCHPTAWDLGKGDFRILMCTKV

Accordingly, in certain embodiments, the foregoing β-hairpin amino acid sequence, or a derivative, or a portion thereof, is used as a binding moiety, or as part of a binding moiety, for detection of SARS-CoV-2.

In additional embodiments, a fusion protein comprising both the α-helix or a derivative or portion thereof) and the β-hairpin or a derivative or portion thereof is used as a binding moiety for detection of SARS-CoV-2. In these embodiments, the α-helix and the β-hairpin sequences can be joined (or are joined) with a flexible linker to generate a fusion peptide that recapitulates the entire viral S-protein binding site (shown in FIG. 8A). In the absence of the virus the helix and hairpin portions of the biosensor are separated, because they do not have sufficient molecular interactions with each other to form a stable complex. However, the helix and hairpin come together upon binding of the viral S-protein, which provides an interface to stabilize the helix/hairpin/S protein ternary complex. Using this type of hybrid binding moiety, the binding event produces a conformational change in the biosensor that is used as a signal transducer to generate a fluorescence readout. For example, if a FRET donor is present on the α-helix and a FRET acceptor is present on the β-hairpin, binding of the biosensor to the S-protein causes the helix and the hairpin to come into sufficient proximity for FRET to occur between the donor and receptor, generating a fluorescent signal. In other embodiments, binding of a hybrid binding moiety to the spike protein causes the binding-induced proximity of the hairpin to the helix to quench the fluorescence of a fluorescent moiety present in the helix portion of the hybrid binding moiety.

Examples 8 and 9 describe the synthesis of two hybrid biosensors containing the α-helix and the β-hairpin of the ACE2 protease domain. A flexible linker is placed between the α-helix and the β-hairpin, and the amino acid sequences of the α-helix and the 3-hairpin are altered to eliminate hydrophobic residues on the face opposite to the binding interface to minimize non-specific binding and/or aggregation, to remove potential fluorescence-quenching residues, and to provide sites for attachment of fluorophores. See FIG. 8A. The two peptide fragments (α-helix and β-hairpin) can be synthesized separately by solid-phase methods. Attachment of sortase recognition sequences to the C-terminus of the helix peptide, and to the N-terminus of the hairpin peptide, allow these two peptides to be joined enzymatically using sortase chemistry. Theile et al. (2013) Nature Protocols 8:1800-1807.

### Fluorescent Moiety

The compositions described herein comprise a fluorescent moiety, or fluorophore, bound (e.g., covalently) to the binding moiety. The fluorescent moieties described herein have the property that their fluorescence is altered by binding of the binding moiety to the target. For certain fluorophores, the fluorescence emission wavelength changes after contact between the binding moiety and the target. In certain embodiments, the emission wavelength shifts to a higher wavelength after binding. For example, the emission wavelength can be increased by at least 10, at least 15, at least 20, at least 25, at least 30, at least 35, at least 40, at least 45, at least 50, at least 60, at least 70, at least 80, at least 90, or at least 100 or more nm (or any integral value therebetween) after binding. In certain embodiments, fluorescence output (e.g., brightness, quantum yield) also increases after binding.

Mechanisms of fluorescence in which emission wavelength shifts to a different wavelength after binding include excited state intramolecular proton transfer (ESIPT). ESIPT fluorophores change their properties depending on the polarity of the environment (i.e., they exhibit solvatochromism). For example, an ESIPT fluorophore will emit fluorescence at a first wavelength when exposed to water, and at a second, different wavelength when buried inside a protein; or when internalized in a protein-protein, protein-peptide or peptide-peptide complex. Thus, in certain embodiments, when a binding moiety/fluorescent moiety composition, as described herein, binds to a target (e.g., a viral particle); the fluorescence emission shifts to a higher wavelength. In additional embodiments, when a binding moiety/fluorescent moiety composition, as described herein, binds to a target (e.g., a viral particle); the fluorescence emission shifts to a lower wavelength.

Thus, in certain ESIPT-based systems, the fluorescence emission has a higher wavelength (and, optionally, a greater intensity) when the biosensor is bound to target, than when the biosensor is unbound. In these embodiments, the fluorescent moiety has the property that its fluorescence emission at two wavelengths is dependent on its environment, such that the ratio of the fluorescence emission at the two wavelengths has a certain value when the binding moiety/fluorescent moiety fusion is free (i.e., not bound to a target), but has a different value when the binding moiety/fluorescent moiety fusion is bound to a target. That is, the ratio of fluorescence emission at the two wavelengths of the fluorescent moiety is altered upon binding to the target. This property of the fluorescent moiety helps reduce problems with the interpretation of signals, providing greater detection accuracy and specificity.

An example of a fluorophore that undergoes ESIPT is 6-N,N-dimethylamino-2,3-naphthalimide (6DMN). 6DMN changes its quantum yield by 120-fold when transferred from water to a hydrophobic environment, and also experiences a shift in emission from orange (λₘₐₓ˜595 nm) in water to cyan (λₘₐₓ˜510 nm) in non-polar solvents. Vasquez et al. (2005) J. Amer. Chem. Soc. 127:1300-1306.

Another fluorophore that undergoes ESIPT is 2-(2-furyl)-3-hydroxychromone (FHC), which has dual emission (λ1max˜430 nm, violet; and λ2max˜530 nm, green) in which the relative intensity of emission at the two wavelengths is highly sensitive to the polarity of the environment. Strizhak et al. (2012) Bioconjugate Chem. 23:2434-2443. Both 6DMN and FHC can be synthesized as F-moc amino acids, and are thereby able to be incorporated into synthetic peptides during solid-phase synthesis. Example 3 describes incorporation of 6DMN and FHC into a peptide corresponding to an α-helix of the ACE2 peptidase domain.

Additional fluorophores that undergo ESIPT are known in the art. See, for example, Sedgwick et al. (2018) Chem. Soc. Rev. 47:8842-8880.

An additional fluorescence mechanism in which the ratio of emission at two wavelengths changes after binding is Forster resonance energy transfer (FRET), also known as fluorescence resonance energy transfer. In FRET, fluorescence emission is altered by proximity between two molecules. The distance between two molecules, at which 50% of total possible energy transfer occurs (i.e., equal intensities of donor and acceptor fluorescence), is known as the Forster distance, or R₀. At shorter distances the transfer efficiency increases, eventually to 100%, and at longer distances it decreases, eventually to 0%. The efficiency of energy transfer depends on the sixth power of the distance, due to the fact that energy transfer is a dipole-dipole interaction. Thus, for example, for a R₀ of 5 nm, the efficiency of energy transfer is over 95% at 3 nm, and approximately 10% at 7 nm. The FRET donor molecule emits at a lower wavelength (higher energy); while the FRET acceptor molecule emits at a higher wavelength (lower energy). Thus, when a FRET donor and a FRET acceptor come within a distance of each other that is close to the Forster radius, emission at the lower wavelength decreases, while emission at the higher wavelength increases.

In one example of a FRET system, the two molecules are Alexa Fluor® 488 (A488, green fluorescence, emission λmax=525 nm) and Alexa Fluor© 594 (A594, red fluorescence, emission λmax=617 nm). The Forster distance (R₀) for this pair of fluorophores is approximately 5 nm.

Any pair of fluorophores in which the emission spectrum of one fluorophore (the FRET donor) overlaps in wavelength with the excitation (absorption) spectrum of the other fluorophore (the FRET acceptor) can be used as a FRET pair. Many such FRET pairs are known in the art.

In additional embodiments, the fluorescence output (e.g., brightness, quantum yield) of a fluorophore decreases after binding of a biosensor to a target. For example, oxazine fluorophores such as Atto 655 emit red fluorescence but can undergo contact quenching by photoinduced electron transfer (PET) from a vicinal electron donor, such as a tryptophan residue in a peptide or protein sequence, or a guanosine residue in a nucleic acid sequence. Because, with fluorophores such as Atto 655, a positive result results in a decrease in signal, in certain embodiments a second fluorophore, having a different emission wavelength, is incorporated at a non-binding portion of the sensor (e.g., N-terminus or C-terminus). A change in the ratio of fluorescence of the first and second fluorophores is then used as the readout.

Other fluorophores that can undergo PET are known in the art. See, for example, de Silva et al. (2009) Analyst 134:2385-2393.

A fluorescent moiety can be attached to a binding moiety by a number of methods. For example, certain fluorophores, such as 6DMN and FHC, can be produced as F-moc amino acid derivatives (by methods known in the art), which can be incorporated directly into a peptide (at a site that will not affect binding of the peptide to its target) during, e.g., solid-phase synthesis. In other embodiments, a maleimide derivative of a fluorophore can be attached to the thiol group of a cysteine residue of a peptide using maleimide chemistry, as is known in the art. In still further embodiments, the non-natural amino acid para-acetylphenylalanine (pAF) is incorporated into a binding moiety during peptide synthesis, or during recombinant protein production (e.g., Wang et al. (2001) Science 292:498-500; Wang et al. (2003) Proc. Natl. Acad. Sci. USA 100:56-61), at a site that will not affect binding of the peptide to its target, and a fluorophore carrying a hydroxylamine (—NH₂OH) group is attached to the pAF residue after synthesis of the peptide, by keto labeling.

### Solid Support

In the detection methods disclosed herein, compositions comprising a binding moiety and a fluorescent moiety (i.e., biosensors) are attached to a solid support. In certain embodiments, the solid support is a sphere or is spheroidal, approximating the shape of a cell. In additional embodiments, a spherical or spheroidal solid support has a diameter commensurate with the size of a cell (e.g., a eukaryotic cell; e.g., a mammalian cell; e.g., a human cell). Thus, for example, to detect a virus that infects human lung epithelium, a solid support will have the approximate size and shape of a human lung epithelial cell.

To this end, spherical or spheroidal solid supports can have a diameter (or a nominal diameter for a spheroidal support) of 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 2.3 μm, 2.4 μm, 2.5 μm, 2.6 μm, 2.7 μm, 2.8 μm, 2.9 μm, 3.0 μm or more, or any fractional value therebetween. In certain embodiments, the diameter (or nominal diameter for a spheroidal support) is greater than 0.5 μm; or is between 0.5 μm and 1.0 μm; or is between 0.2 and 0.8 μm, or is between 0.3 and 0.7 μm, or is between 0.4 and 0.6 μm, or is between 0.5 μm and 1.5 μm; or is between 1.0 μm and 1.5 μm or is between 0.8 μm and 1.2 μm or is between 0.9 μm and 1.1 μm.

In certain embodiments, the binding moiety corresponds to a protein, or portion of a protein, that serves as the cell-surface receptor for the target; e.g., the binding moiety is a protein (or a peptide sequence from a protein), that is a viral receptor on the surface of a cell, or a portion thereof. Accordingly, for optimal sensitivity and specificity in detecting a virus that infect a particular cell, the density of the biosensor on the solid support corresponds to the density of the viral receptor on the surface of the cell. Cell-surface receptors for various viruses are known in the art, as are methods for estimating the density of viral receptors on the surface of the cell. An exemplary method for determining the density of the ACE2 protein (i.e., the receptor for SARS-CoV-2) is presented in Example 6.

Materials for use as a solid support can be any solid known in the art to which a biosensor can be attached. Exemplary materials for use as a solid support include, but are not limited to, polystyrene, polyethylene, polypropylene, glass, and magnetic materials. In certain embodiments, the solid support is transparent to light. In other embodiments, the solid support is opaque.

### Solid Supports Comprising Biosensors (Sensor Beads or “Host Decoys”)

Practice of the detection methods disclosed herein utilizes compositions (sensor beads or “cell decoys”) comprising a biosensor (i.e., binding moiety and fluorescent moiety) affixed to a solid support (e.g., the surface of a bead). Such cell decoys interact with targets (e.g., viral particles) by multivalent binding and their fluorescence output (e.g., wavelength, intensity) is altered in response to said binding. Amplification of the binding affinity and specificity (e.g., for a viral particle) can thus be engineered by controlling the density of biosensor molecules coating the support. Example 6 provides sample calculations for determining coverage density of biosensors containing the ACE2 α-helix on a 1 μm polystyrene bead that will provide maximal sensitivity. The same formulae can be used for determining coverage density for other binding moieties, providing that the monovalent dissociation constant of the complex between the binding moiety and the target molecule is known.

A biosensor can be attached to a solid support by functionalization of the solid support with a reactive moiety that is capable of being bound to the support and being covalently bound to a component of a protein or peptide, such as, for example, an amino group, a carboxyl group, a sulfhydryl group, a phenyl group, a phenolic group, a hydroxyl group, an imidazolium group, a guanidine group or an amide. For example, solid supports functionalized with amino groups or carboxylic acid groups can be used for attachment of proteins or peptides at their C-terminus or N-terminus, respectively. Additional methods and reagents for attachment of peptide amino groups or carboxyl groups to various other functional groups are known in the art.

Another method for attaching a biosensor to a solid support is to use a biotinylated binding moiety and a biotinylated support; in which case the binding moiety is associated to the support using avidin or streptavidin as linker. Methods for in vitro biotinylation of proteins and peptides are well established, and protein biotinylation can also be performed in vivo during recombinant protein expression. In vivo protein biotinylation is particularly useful when the binding moiety cannot be produced by solid-phase synthesis (e.g., because it too large and needs to be made recombinantly). A protein biotinylated in vivo can be associated to a support using avidin or streptavidin as linker.

In additional embodiments, the binding moiety is biotinylated and the support is functionalized with avidin or streptavidin. In further embodiments, the support is biotinylated and the binding moiety is functionalized with avidin or streptavidin. In either of these cases, attachment of the binding moiety to the support is mediated by avidin-biotin or streptavidin-biotin interactions.

Association of a biotinylated moiety to a moiety containing avidin or streptavidin occurs in water or aqueous buffers at ambient temperature.

Any number of biosensors can be attached to a solid support. The number of biosensors attached to a solid support (the coverage density) can be adjusted depending on the degree of sensitivity required, with higher coverage density enabling higher sensitivity of detection, as described elsewhere herein. In certain embodiments, the coverage density is between 100 and 50,000 biosensors per solid support. In additional embodiments, a solid support contains between about 500 to about 50,000 biosensors. In additional embodiments, a solid support contains between about 500 to about 40,000 biosensors. In additional embodiments, a solid support contains between about 500 to about 30,000 biosensors. In additional embodiments, a solid support contains between about 500 to about 25,000 biosensors. In additional embodiments, a solid support contains between about 500 to about 20,000 biosensors. In additional embodiments, a solid support contains between about 500 to about 15,000 biosensors. In additional embodiments, a solid support contains between about 500 to about 10,000 biosensors. In additional embodiments, a solid support contains between about 500 to about 5,000 biosensors. In additional embodiments, a solid support contains between about 500 to about 2,500 biosensors. In additional embodiments, a solid support contains between about 500 to about 1,500 biosensors. In additional embodiments, a solid support contains between about 500 to about 1,000 biosensors.

One of the factors contributing to the sensitivity and specificity of detection, using the methods and compositions disclosed herein, is that a solid support comprising biosensors can be designed to approximate the size, shape and receptor density of the host cell (hence the term “cell decoy”). Approximating the size and shape of the host cell is accomplished by choosing the size and shape of the solid support. For example, spherical (and spheroidal) polystyrene beads (as well as beads made of other materials) are available in a variety of sizes, such that it is possible to choose the size of the solid surface to be roughly the size of the host cell. Thus, binding of the target to the cell decoy occurs under steric conditions that are similar to binding of the target to its host cell. Moreover, as noted elsewhere herein, the density of the biosensor on the solid support can be adjusted to be similar to the density of the receptor for the target on the surface of the host cell. Thus, the cell decoys disclosed herein are designed to recreate the size, shape and receptor density of the host cell, thereby providing multivalent binding to the target on a surface that resembles the host cell.

### Detection Systems—Multiwell Plates

In certain embodiments for detection of biological targets, biosensor-containing solid supports (i.e., cell decoys or sensor beads) are dispersed on a surface. In additional embodiments, the surface is transparent to light. Exemplary surface materials include, but are not limited to, polyethylene, polyethylene terphthalate, polypropylene, polystyrene, glass, plexiglass, acrylic (e.g., polymethacrylate), polycarbonate and cellulose acetate. Additional surface materials are known in the art.

In further embodiments, the surface is the bottom of a microwell plate. In these embodiments, the cell decoys can lie freely on the surface, or they can be affixed to the surface. Methods for affixing solid supports (e.g., beads) to surfaces are known in the art and include, for example avidin-biotin systems and streptavidin-biotin systems. For example, in one embodiment, avidin- or streptavidin-functionalized beads are attached to biotin-coated microwell plates. The beads are suspended in water or an aqueous buffer and the suspension is added to the microwell and incubated for one hour at ambient temperature. Unbound beads are removed by aspiration. In an additional embodiment, biotin-functionalized beads are attached to avidin- or streptavidin-coated microwell plates. The beads are suspended in water or an aqueous buffer and the suspension is added to the microwell and incubated for one hour at ambient temperature. Unbound beads are removed by aspiration. In a further embodiment, biotin-functionalized beads are joined to biotin-coated microwell plates by avidin or streptavidin. Affixing the beads to the plate is accomplished by suspending the beads in water or an aqueous buffer, adding the suspension to the microwell, and incubating in the presence of avidin or streptavidin, with avidin or streptavidin being present at a ratio of about 10-50 avidin or streptavidin molecules per bead, for one hour at ambient temperature. As in the previous embodiments, unbound beads can be removed by aspiration.

Attachment of supports to the surface facilitates direct excitation and unblocked collection of emitted fluorescence from the surface through the transparent bottom of the surface. Inverted excitation and fluorescence collection together with a sparse microarray of sensor beads minimizes background, scattering and absorption from the sample to increase the sensitivity of the assay.

Changes in fluorescence properties are measured using any type of fluorescence analyzer, e.g., a fluorometer. In certain embodiments, fluorescence is analyzed using a plate reader fluorescence imager, which is essentially a combination of a well plate reader, a fluorescence excitation source and an optical microscope. Using a plate reader fluorescence imager, separate images of individual beads can be obtained separately. Being able to analyze the individual fluorescence of each bead, rather than the total fluorescent signal from a plurality of beads, greatly reduces background, leading to a significant increase in sensitivity of detection.

Fluorescence microscopes with plate-reading capability (i.e., plate reader fluorescence imagers) capable of single-bead resolution, are available commercially and include ImageXpress Pico (Molecular Devices, San Jose, CA), Cytation 5 (BioTek Instruments, Santa Clara, CA), Spark Cyto (TECAN US, Morrisville, NC), and Nyone (Advanced Robotics, Tarzana, CA, Synentec GmbH, Elmshorn, Germany). These devices are equipped with software for analysis of fluorescence intensity and automated particle counting. In certain embodiments, code will be added to that software (using, e.g., Matlab) to interpret the results; e.g., to convert fluorescence signal to viral load.

In certain embodiments, a fluorometer or plate-reading fluorescence imager, for use in the practice of the methods disclosed herein, has capacity for inverted excitation and detection. By exciting and collecting fluorescence from below the sample, background fluorescence is minimized.

Background is also controlled by minimizing the density of the supports on the surface. Accordingly, in certain embodiments, the density of the solid support per unit area of the surface is between one support per 320 μm² and one support per 32 mm² of the surface (e.g., for a 96-well microtiter plate. In other embodiments, the density of the solid support per unit area of the surface is between one support per 112 μm² and one support per 11.2 mm² of the surface (e.g., for a 384-well microtiter plate. In additional embodiments, the density of the solid support per unit area of the surface is between one support per 100 μm² and one support per 10 μm² of the surface, the density of the solid support per unit area of the surface is between one support per 500 μm² and one support per 5 μm² of the surface, or the density of the solid support per unit area of the surface is between one support per 1,000 μm² and one support per 20 μm² of the surface.

In certain embodiments, in which the support is a polystyrene bead and the surface is the bottom of a microwell, the number of supports on the surface is 100 or more; 500 or more; 1,000 or more; 2,500 or more; 5,000 or more; 7,500 or more; 10,000 or more; 15,000 or more; 20,000 or more; 30,000 or more; 40,000 or more; 50,000 or more, 60,000 or more; 70,000 or more; 80,000 or more; 90,000 or more or 1000,000 or more. In additional embodiments, the number of supports on the surface is between 1,000 and 5,000; between 5,000 and 10,000; between 10,000 and 15,000; between 15,000 and 20,000; between 10,000 and 20,000; between 5,000 and 15,000; between 7,500 and 12,500; between 9,000 and 11,000; between 10,000 and 50,000; between 10,000 and 25,000; between 25,000 and 50,000; between 20,000 and 40,000; or between 1,000 and 50,000. In further embodiments, the number of supports on the surface is between 50,000 and 150,000; between 60,000 and 140,000; between 70,000 and 130,000; between 80,000 and 120,000; between 90,000 and 110,000 or between 95,000 and 105,000. In still more embodiments, the number of supports on the surface is between 25,000 and 75,000; between 30,000 and 70,000; between 35,000 and 65,000; between 40,000 and 60,000 or between 45,000 and 55,000.

### FACS-Based Detection Systems

Sensor beads can also be analyzed individually using, for example a fluorescence-activated cell sorter. In these methods, sensor beads that have not been exposed to a test sample are assayed to provide baseline levels of the first and second emission properties (e.g., wavelengths). Sensor beads that have been contacted with a sample are then analyzed; with an increase in the second property being indicative of the presence of the target. FACS devices are known in the art and these devices are equipped with software for analysis of fluorescence. In certain embodiments, code will be added to that software (using, e.g., Matlab) to interpret the results; e.g., to convert fluorescence signal to viral load.

### Fiber Optic-based Detection Systems

Another method for analyzing fluorescence of single beads is by using bundled fiber optic-based systems. See, for example, Orth et al. (2019) Science Advances 5(4):1555 and Flusberg et al. (2005) Nature Methods 2:941-950. Software (e.g., Matlab) for converting recorded fluorescence signal from any of the systems described herein into a diagnostic result can be run on any platform, including a smartphone app.

### Methods

The compositions and detection systems described above are used in methods for detecting a target (e.g., a biological target such as a virus). To that end, a sample (see below) is contacted with a sensor bead (e.g., in a microplate well, a micro test tube, a capillary, or any other suitable container) and incubated in water or a physiological buffer (e.g., PBS, Ringer's, or any buffer compatible with the stability of the sample) for a time and at a temperature suitable for binding between the binding moiety and the target molecule. In certain embodiments, sensor beads are assayed for a first emission property prior to their contact with a sample. In certain embodiments, contact is conducted at ambient temperature. In other embodiments, contacting is conducted at body temperature (i.e., the body temperature of the subject from whom the sample was obtained). The time of contact can be one second, 2 seconds, 5 seconds, 10 seconds, 30 seconds, one minute, two minutes, 5 minutes, 10 minutes, 20 minutes, 30 minutes, 45 minutes, one hour, or any integral value between one and 60 seconds or between one and 60 minutes.

Following contact between the sensor bead and the sample under conditions of time, temperature and buffer composition appropriate for binding, sensor beads are analyzed for their fluorescence, using one of the systems described above (plate reader fluorescence imagers, FACS or fiber optic), or any other fluorescent detection system capable of resolving individual sensor beads, to obtain a second emission property. Controls can include sensor beads that have not been contacted with sample, from which a first emission property can be measured. Changes in fluorescence (i.e., differences between a first emission property and a second emission property) are determined and converted to a result (e.g., viral load) by methods known in the art (e.g., a standard curve).

### Samples and Sample Preparation

Any type of biological sample that can be obtained from a subject is suitable for testing using the methods and compositions disclosed herein. For example, respiratory tract samples, such as nasopharyngeal swabs, throat swabs, buccal swabs, mucus, saliva and sputum can be used. Additional sample types include, without limitation, blood, plasma, serum, cerebrospinal fluid, lymph, urine, stool and semen.

Saliva samples can be analyzed directly with no further preparation. For example, a small volume (e.g., 0.3 ml) is collected with a micropipette and transferred to a well in the detection device.

Certain types of sample may require one or more sample preparation steps. For example, swab samples (e.g., nasopharyngeal swabs, throat swabs, buccal swabs) are resuspended in a liquid solution (e.g., viral transport medium (VTM), Amies transport medium, or sterile saline solution) by physical methods (e.g., agitation, stirring), and a small volume (e.g., 0.3 ml) of the liquid is collected and transferred to a well in the detection device.

### Detection of SARS-CoV-2

In one embodiment, the methods and compositions disclosed herein are used for performing diagnostic and screening tests; i.e., measurement of a specimen for the purpose of diagnosis, prevention, or treatment of COVID-19, or for the assessment of health or impairment of health of an individual COVID-19 patient.

The target for this particular embodiment of the assay is the set of clinically relevant strains of SARS-CoV-2 that demonstrate infectivity to humans by adhesion to the human ACE-2 receptor. The system detects any type of coronavirus particles that bind with high affinity to the ACE-2 receptor and thus retain infectivity, including wild-type virus and any viruses that have undergone changes due to mutation or genetic drift of the viral genome. Importantly, by mimicking the first step in host cell infection, the test can distinguish between infectious and non-infectious (inactive) viral particles. Using the binding moieties described in the Examples infra, no cross reactivity is expected from any human protein or from any other microorganism, with the exception of other coronaviruses that use the same binding site on ACE-2 for infection (i.e., SARS-CoV). The test are designed to detect viral loads in the clinically relevant range of 10³ to 10⁸ SARS-CoV-2 copies per milliliter of saliva. To et al. (2020) Clinical infectious Diseases 71:841-843.

Samples for the test are, for example, specimens of saliva, sputum, nasopharyngeal swabs or oral swabs, collected from a patient (for diagnostic purposes) or person (for screening purposes) and prepared as a liquid sample. Oral and nasopharyngeal swabs require one physical step to transfer viral particles from the swab into solution, as described in the section herein entitled “Samples and Sample Preparation.” Saliva specimens are easily collected, requiring only that a patient expectorate into a sterile bottle. The minimal manipulation involved in the collection of saliva greatly minimizes the chance of exposing healthcare workers to infection.

Studies have demonstrated a high concordance (>90%) between saliva and nasopharyngeal specimens in the detection of respiratory viruses, including coronaviruses. To et al. (2019) Clin. Microbiol. & Infection 25:372-378. SARS-CoV particles are present in saliva at high titers (Wang et al. (2004) Emerging Infectious Diseases 10:1213), as are SARS-CoV-2 particles. To et al. (2020) supra. Saliva samples have recently been reported to provide higher sensitivity and consistency for COVID-19 diagnostics than nasopharyngeal samples. Wyllie et al. (2020) Medrxiv doi: https://doi.org/10.1101/2020.04.16.20067835.

Before sample application, a calibration measurement is performed to establish a blank baseline to be used as reference for the analysis of the fluorescence emission profile of the samples. Calibration is achieved by measuring the fluorescence emission of the device (i.e., sensor bead) in the absence of sample. After recording the blank, each sample is applied to a sample container, such as one well of a multi-well plate (e.g., a 96-, 384- or 1536 well-plate) derivatized with the SARS-CoV-2 sensor bead microarray on the bottom surface of the wells. This step can be carried out semi- or fully automatically using an electronic multi-channel micropipette injector or a high-throughput titration robot. Sample delivery for non-batch configurations (microfluidics or FACS versions of the device) are adjusted according to their technical characteristics.

For measurement and analysis, the device containing the loaded samples is subjected to individualized measurement of the fluorescence emission properties of each sample. The measurement consists of the recording of fluorescence emission in two colors/channels (e.g., green and red). Individualized sample measurements are obtained using (1) a spectrofluorometer equipped with a plate reader (with inverted excitation and emission), (2) a high-resolution fluorescence imaging fiber-optic bundle for microfluidics systems, or (3) bead by bead on a flow cytometer (FACS version). The recorded fluorescence is referenced to a blank measurement run on the same device prior to addition of the sample (or, for FACS-based systems, on sensor beads that have not been in previous contact with samples). The ratio between red and green fluorescence after blank subtraction is compared to an instrument calibration curve (performed at time of installation using known sample standards) to provide a direct, quantitative determination of the number of functional (infective) viral particles present in the sample. Green fluorescence provides an internal reference for the emission profile of the sensor bead microarray; and depends on number of beads per m² and the number of biosensor molecules coating each bead. Red fluorescence is observed upon adhesion of viral particles to sensor beads. The observed ratio of red to green fluorescence on each bead, compared to the calibration curve, provides a direct measurement of the number of viral particles present in the sample.

Sensor bead Matrices

Also provided herein are methods for determining properties of a sensor bead (e.g., size and biosensor density) that will provide optimal ranges for detection of a particular target. In these embodiments, a matrix of solid supports is constructed (e.g., in the wells of a microwell plate). In one dimension, the size (e.g., diameter) of the support is varied and in the other dimension, the density of the biosensor on the support is varied. The matrix is contacted with a target, and the point on the matrix providing the highest signal identifies the size and biosensor density that will provide maximal sensitivity of detection.

In additional embodiments, arrays can be used to quantitate the amount of a target (e.g., determine the viral load in a subject). In these embodiments, sensor beads containing different densities of biosensor are arrayed along an axis of the matrix. Because the viral load in subjects with COVID-19 varies so widely (10³-10⁸ viral particles per ml of saliva), this type of matrix will ensure that one or more sensor beads in the array has a sufficient biosensor density to detect a low virus concentration, while other sensor beads in the array have a biosensor density that will not be saturated by a high virus concentration.

### Multiplex Systems

Detection of two or more targets is possible using multiplex systems. In these systems, two (or more) different binding moieties, each specific to a different target, are used; with each binding moiety having a different fluorophore, so that fluorescence emission will be distinguishable for each target. Both binding moieties can be present on the same sensor bead, or each different binding moiety can be present on a separate bead.

### ADDITIONAL EXAMPLES

### Example 5: Selection of Regions of the ACE2 Receptor Peptidase Domain for Use as Binding Moieties

The human cellular receptor for the SARS-CoV-2 virus is the cell surface enzyme angiotensin converting enzyme 2 (ACE2). The amino acid sequence of ACE2 is shown below and at FIG. 11. The atomic structure of the complex between the receptor binding domain (RBD) of the SARS-CoV-2 spike protein (S protein) and the ACE2 protein has recently been solved. Yan et al. (2020) Science 367(6485):1444-1448. The structure of the complex reveals that the receptor binding domain of the SARS-CoV-2 spike protein interacts with the peptidase domain (PD) of ACE2, with a binding affinity of ˜5 nM. Walls et al. (2020) Cell 181:281-292. MSSSSWLLLS LVAVTAAQST IEEQAKTFLD KFNHEAEDLF YQSSLASWNY NTNITEENVQ NMNNAGDKWS AFLKEQSTLA QMYPLQEIQN LTVKLQLQAL QQNGSSVLSE DKSKRLNTIL NTMSTIYSTG KVCNPDNPQE CLLLEPGLNE IMANSLDYNE RLWAWESWRS EVGKQLRPLY EEYVVLKNEM ARANHYEDYG DYWRGDYEVN GVDGYDYSRG QLIEDVEHTF EEIKPLYEHL HAYVRAKLMN AYPSYISPIG CLPAHLLGDM WGRFWTNLYS LTVPFGQKPN IDVTDAMVDQ AWDAQRIFKE AEKFFVSVGL PNMTQGFWEN SMLTDPGNVQ KAVCHPTAWD LGKGDFRILM CTKVTMDDFL TAHHEMGHIQ YDMAYAAQPF LLRNGANEGF HEAVGEIMSL SAATPKHLKS IGLLSPDFQE DNETEINFLL KQALTIVGTL PFTYMLEKWR WMVFKGEIPK DQWMKKWWEM KREIVGVVEP VPHDETYCDP ASLFHVSNDY SFIRYYTRTL YQFQFQEALC QAAKHEGPLH KCDISNSTEA GQKLFNMLRL GKSEPWTLAL ENVVGAKNMN VRPLLNYFEP LFTWLKDQNK NSFVGWSTDW SPYADQSIKV RISLKSALGD KAYEWNDNEM YLFRSSVAYA MRQYFLKVKN QMILFGEEDV RVANLKPRIS FNFFVTAPKN VSDIIPRTEV EKAIRMSRSR INDAFRLNDN SLEFLGIQPT LGPPNQPPVS IWLIVFGVVM GVIVVGIVIL IFTGIRDRKK KNKARSGENP YASIDISKGE NNPGFQNTDD VQTSF (SEQ ID NO:22)

Although the SARS-CoV-2 spike protein and ACE2 are large proteins and have sophisticated structural patterns; the atomic coordinates of their complex reveal that the binding interface between these two proteins comprises just two, relatively short segments of the PD domain of ACE-2 interacting with the RBD of the viral S protein. One of these segments is an α-helix (the al helix) and the other is a β-hairpin (FIG. 8A). The binding interface is defined by a combination of hydrophobic interactions (mediated by aliphatic and aromatic side chains) that provide affinity, and a large number of side-chain hydrogen bonds that provide binding specificity.

A 23-residue peptide encompassing the central portion of the al-helix of the ACE2 PD domain has recently been shown to bind to the S1 protein RBD with an affinity of ˜1 μM. Zhang et al. (2020) bioRxiv 10.1101/2020.03.19.999318. A designed fragment based on the full α-1 helix, stabilized with the support of the α-2helix, binds the RBD with an affinity of ˜50 nM (i.e., only 10-fold lower than the binding affinity of the entire ACE2 receptor protein). Romano et al. (2020) bioRxviv 10.1101/2020.04.29.067728. The al-helix was therefore identified as a minimal high-affinity target for the spike protein. The amino acid sequence of this α-helical region is:

(SEQ ID NO: 15)
STIEEQAKTFLDKFNHEAEDLFYQSSLASWNYNTNITE
or
(SEQ ID NO: 5)
STIEEQAKTFLDKFNHEAEDLFYQSSLASWNYNTN

Construction of a binding moiety comprising this helix is described in Examples 6 and 7.

Upon binding of the ACE2 PD to the spike protein RBD, the β-hairpin of the ACE2 PD fills a wedge between the α-helix and the spike protein that should enhance binding cooperativity. The amino acid sequence of this β-hairpin region is: KAVCHPTAWDLGKGDFRILMCTKV (SEQ ID NO:6) Because the β-hairpin sequence is expected to enhance binding affinity between the α-helix and the RBD of the S protein; a binding moiety comprising both the α-helix and the β-hairpin was also constructed. See Examples 9 and 10. This binding moiety is expected to have higher affinity and specificity for SARS-CoV-2 S protein than the α-helix alone.

### Example 6: Binding Moiety Containing α-Helix of ACE2 Peptidase Domain

For use in detecting SARS-CoV-2 by binding to the RBD of its spike protein, the amino acid sequence of the al-helix is modified by (1) attaching a linker sequence GSGSG to the amino terminus, (2) replacing the phenylalanine residue at position 14 of the helix sequence given above in Example 1 with alanine, (3) replacing the histidine residue at position 16 with a fluorophore (see Example 7), and (4) replacing the phenylalanine residue at position 22 with alanine. Replacement of the phenylalanine residues at positions 14 and 22 is done to reduce the hydrophobicity of the helix on its non-interacting face, thereby minimizing non-specific binding and/or aggregation of the binding moiety. Accordingly, the amino acid sequence of an α-helix peptide binding moiety used for detection of SARS CoV-2 is: GSGSGSTIEEQAKTFLDKANCEAEDLAYQSSLAS (SEQ ID NO:12) or GSGSGSTIEEQAKTFLDKANCEAEDLAYQSSLASWNYNTN (SEQ ID NO:7); where a fluorophore can be attached via cysteine in between N and E at position 21.

The peptide is synthesized by solid-phase peptide synthetic methods.

### Example 7: Insertion of Fluorophore into α-Helix Binding Moiety

6-N,N-dimethylamino-2,3-naphthalimide (6DMN) and 2-(2-furyl)-3-hydroxychromone (FHC) are fluorophores that undergo excited state intramolecular proton transfer (ESIPT). They are synthesized as F-moc amino acids. See, for example, Vizquez et al. (2005) J. Amer. Chem. Soc. 127:1300-1306 (for synthesis of F-moc-6DMN) and Strizhak et al. (2012) Bioconjugate Chem. 23:2434-2443 (for synthesis of F-moc-FHC). The F-moc derivatives are incorporated into the α-helix peptide (as shown in Example 6, above) during the solid-phase synthesis at position 21 (indicated by X). This position is completely solvent exposed in absence of virus, thus providing very low fluorescence emission. Upon binding of the α-helix binding moiety to the S protein RBD, the binding moiety becomes covered by the viral S-protein, resulting in brighter emission and a spectral shift (solvatochromism). Computer modeling indicates that the fluorophore will not interfere with the binding between the peptide and the spike protein, but rather will enhance binding through additional hydrophobic interactions.

Thus, formation of a complex between the α-helix and the RBD of the SARS-Cov-2 spike protein will drastically change the environment of the fluorophore; providing a sensitive, very high gain readout with a blue shift (6DMN) or a red shift (2-FHC) and an increase in emission intensity by up to 120-fold.

This design is simple to synthesize and highly sensitive, inasmuch as it is much easier to detect very low fluorescence without any background than to detect a small change in the intensity of a large signal (as would be the case for a system with a high background).

### Example 8: PET-Based Binding Moiety Containing α-Helix and β-Hairpin of ACE2 Peptidase Domain

A hybrid peptide binding moiety, containing both the α-helix and the 0-hairpin of the ACE2 PD joined by a flexible linker, is constructed. This construct contains the entire binding domain for the SARS-CoV-2 spike protein (i.e., both the α-helix and the 0-hairpin). The two portions of this binding moiety (helix and hairpin) are synthesized separately by solid-phase peptide synthesis, then joined enzymatically by the sortase reaction. The α-helix portion of this hybrid binding domain is similar to the α-helix peptide used for the ESIPT probe described in Examples 6 and 7, with Phe14 of the helix sequence shown in Example 5 substituted by Ala, no change at His 16 of the helix sequence shown in Example 1, Phe22 of the helix sequence shown in Example 5 substituted by Cys to provide a site for attachment of the fluorophore (see below), and Trp30 of the helix sequence shown in Example 5 substituted with tyrosine, to remove a potential (binding unspecific) PET donor. The N-terminal linker sequence (SSGSG) is slightly different from that present in the al-helix binding domain described in Examples 6 and 7. Finally, a sortase recognition sequence (LPETG) is present at the C-terminus of this peptide. Accordingly, the amino acid sequence of the helical portion of the PET binding moiety is:

(SEQ ID NO: 16)
SSGSGSTIEEQAKTFLDKANHEAEDLCYQSSLASYNYNTNLPETG

Subsequent to synthesis, a fluorophore (e.g., Atto 655) is incorporated into the peptide at position C27 by maleimide chemistry. First, the C27 residue of the peptide is reduced by treatment with an equimolar amount of tris(2-carboxyethyl)phosphine (TCEP). TCEP is then removed by gel filtration chromatography. An equimolar amount of the maleimide derivative of Atto 655 (commercially available, e.g., from Sigma-Aldrich, St. Louis, MO) is added to the cysteine-reduced peptide and incubated in 50 mM PBS, 150 mM NaCl, pH 7.3 in the dark, either overnight at 4° C. or for 4 hours at ambient temperature.

The β-hairpin portion of the peptide is modified to eliminate hydrophobic residues on the face opposite to the binding interface, minimizing the possibility of non-specific binding and/or aggregation, by substituting Phe16 of the hairpin sequence shown in Example 5 with Gln, and substituting Ile 18 of the hairpin sequence shown in Example 5 with Thr. Two glycine residues are appended to the N-terminus of this peptide so that it will participate as a substrate in the sortase reaction. Accordingly, the amino acid sequence of the hairpin portion of the PET binding moiety is:

(SEQ ID NO: 17)
GGKAVCHPTAWDLGKGDQRTLMCTKV

Subsequent to their synthesis, and the labeling of the helical peptide with a fluorophore, the two portions of the binding moiety (helix peptide and hairpin peptide) are joined in a sortase reaction. Theile et al., supra. Briefly, the peptides are exchanged into sortase buffer (50 mM Tris, pH 7.5, 150 mM NaCl) by gel filtration. The reaction mixture contains 50 μM of the N-terminal helix peptide, 150-300 μM of the C-terminal hairpin peptide, 10 mM CaCl₂), and 5 μM sortase. The reaction is conducted overnight in the dark at ambient temperature. The ligated peptide product (see below) is purified by FPLC using a His-Trap Excel column, eluting with an imidazole gradient up to 500 mM. The ligation product can be further purified on FPLC using analytical scale SEC.

Following joining by sortase, the hybrid binding domain has the amino acid sequence shown below with Atto 655 conjugated to residue C27:

(SEQ ID NO: 18)
SSGSGSTIEEQAKTFLDKANHEAEDLCYQSSLASYNYNTNLPETGGGKAV
CHPTAWDLGKGDQRTLMCTKV

This hybrid binding domain is designed to generate a fluorescent readout based on contact quenching of an oxazine fluorophore (e.g., a red-emitting fluorophore such as Atto 655) by photoinduced electron transfer (PET) from a tryptophan residue in the protein. PET occurs when the electron acceptor (e.g., an oxazine fluorophore in its excited state) is within molecular contact (<1 nm) of the donor (tryptophan). At distances shorter than 1 nm, the fluorescence is fully quenched (no emission) whereas at distances greater than about 1 nm, quenching does not take place.

Binding of the viral S protein to the α-helical portion of the biosensor induces the β-hairpin to fold over, inserting itself into the cleft formed by the complex of the α-helix and the viral S protein, to form a ternary complex of S Protein, α-helix and p-hairpin. Formation of the ternary complex brings the Trp residue at position 56 of the hybrid binding domain sequence shown above within PET distance of the Atto 655 present at residue C27 of the hybrid binding domain sequence shown above, thereby quenching its fluorescence.

The advantages of a PET readout are that the phenomenon has (1) a very short distance dependence (1 nm), which increases its molecular specificity, and (2) a binary response (on or off) that maximizes the signal gain. Potential disadvantages are: 1) the phenomenon is not ratiometric (i.e., it relies on only a single wavelength of fluorescence), and 2) the positive result (binding of the viral particle) results in a loss of signal, which could lead to false positive results.

To minimize these potential disadvantages, a two-color approach is used in which a second dye (e.g., Alexa 488, green fluorescence) is incorporated into the biosensor in a position insensitive to the binding of the virus (e.g., the N-terminus), so that fluorescence of the second dye can be used as internal calibration. In this approach, the biosensor is synthesized as described above, with the addition of attachment of the internal control fluorophore (e.g., Alexa 488) to the N-terminus of the α-helix peptide during solid-phase synthesis, e.g., as a F-moc derivative, prior to sortase ligation of the two peptides.

### Example 9: FRET-Based Binding Moiety Containing α-Helix and β-Hairpin of ACE2 Peptidase Domain

A second hybrid binding domain, containing both the α-helix and the 0-hairpin of the ACE2 PD joined by a flexible linker, is also constructed for use in a FRET-based assay. In the α-helical portion of this construct, Ala18 of the helix sequence presented in Example 5 is substituted with para-acetyl phenylalanine (pAF) for attachment of fluorophore; Phe 22 of the helix sequence presented in Example 5 is substituted with alanine, to reduce the possibility of non-specific hydrophobic interactions with target; and Trp 30 of the helix sequence presented in Example 5 is substituted with tyrosine. The linker SSGSG is present at the N-terminus of the peptide, and the sortase recognition sequence LPETG is present at the C-terminus of the peptide. Accordingly, the amino acid sequence of the helical peptide for the FRET-based biosensor is:

(SEQ ID NO: 19)
SSGSGSTIEEQAKTFLDKFNHE(pAF)EDLAYQSSLASYNYNTNLPETG

As with the PET biosensor, the helical portion of this FRET-based biosensor is manufactured by solid-phase peptide synthesis. pAF is available as a F-moc amino acid and can be used directly in the solid phase synthetic method. Subsequent to synthesis of the peptide, a hydroxylamine derivative of Alexa 594 (commercially available from Thermo Fisher Scientific) is introduced at the pAF residue by keto-labeling. For this reaction, a 5-fold molar excess of hydroxylamine-derivatized Alexa 594 is combined with the peptide in 50 mM sodium acetate, 150 mM NaCl, pH 4; and the mixture is incubated in the dark for 72 hours at 37° C.

In the 0-hairpin portion of this FRET-based binding domain, the naturally-occurring hairpin sequence is modified by inserting a Cys residue (for attachment of fluorophore) between Phe16 and Arg17 of the hairpin sequence presented in Example 5; and substituting Ile18 of the hairpin sequence presented in Example 11 with Thr, to minimize non-specific binding and/or aggregation by eliminating a hydrophobic residue on the face opposite to the binding interface. In addition, the sortase recognition sequence GG is present at the N-terminus of the peptide. Accordingly, the amino acid sequence of the hairpin peptide portion of the FRET-based biosensor is:

(SEQ ID NO: 20)
GGKAVCHPTAWDLGKGDFCRTLMCTKV

Subsequent to synthesis of the peptide, a maleimide derivative of Alexa 488 is conjugated to the inserted Cys residue by maleimide chemistry, as described in Example 8.

It should be noted that, for double labeling of a peptide or protein in which one of the labels is attached to a cysteine residue; thiol labeling of the cysteine should precede keto labeling.

Following the synthesis of the individual peptides, and incorporation of fluorophores therein; the fluorophore-labeled peptides are joined in a sortase reaction (as described in Example 8) to generate a biosensor having the amino acid sequence shown below with Alexa 594 conjugated to pAF in the helical portion, and Alexa 488 conjugated to the Cys residue inserted into the hairpin portion of the peptide.

(SEQ ID NO: 21)
SSGSGSTIEEQAKTFLDKFNHE(pAF)EDLAYQSSLASYNYNTNLPETGG
GKAVCHPTAWDLGKGDFCRTLMCTKV

This biosensor design contains two fluorophores, one (Alexa 594, light red fluorescence, energy acceptor) in the α-helical segment of the hybrid biosensor peptide, and the other (Alexa 488, pale green/cyan fluorescence, energy donor) in the β-hairpin segment of the biosensor peptide. The two fluorophores are chosen to be a Forster resonant energy transfer pair. In the absence of virus, the two segments are far enough apart that the distance between the fluorophores is longer than the characteristic R₀, which results in low FRET efficiency (i.e., higher green intensity). Upon binding to the virus, the RBD-helix-hairpin ternary complex is formed, causing the distance between the fluorophores to become shorter than R₀, resulting in enhanced energy transfer and thus an increase in red emission over green.

The advantages of FRET are its intrinsically ratiometric nature (the signal depends on the ratio of the red and green intensities), and its distance dependence, which makes the signal (FRET efficiency change) function as molecular ruler. A potential disadvantage is its limited gain, as FRET changes in fluorescence intensity produced by protein conformational rearrangements are normally between 0.2 and 0.8.

### Example 10: Functionalization of Polystyrene Beads with Biosensors

Micron-sized polystyrene beads prepared for protein functionalization and surface immobilization are routinely used for single molecule biophysical experiments and are commercially available (e.g., Spherotech Inc., Lake Forest, IL) with different sizes and functionalization chemistry. Aminated beads or carboxylated beads are uniformly functionalized with either amino or carboxylic acid groups that can be used to covalently link the biosensor peptide through its free N-terminal amino group or its C-terminal carboxyl group.

For attaching a biosensor peptide to carboxylated beads, as is done for the biosensors described in Examples 6, 7, 8, and 9 biosensor peptide and carboxylated beads are combined in the presence of 1-ethyl-3(-3-dimethylaminopropyl) carbomiimide hydrochloride (EDC, Sigma Chemical Cat. No. E7750) in an aqueous solution at neutral pH (e.g., 50 mM PBS, 150 mM NaCl, at a pH of 7.0-7.5 or 50 mM Tris, 150 mM NaCl, at a pH of 7.0-7.5) and incubated at ambient temperature for two hours. Concentrations of the biosensor and the beads will depend on the desired coating density which can be determined, for example, as described in Example 11. Coated beads are then separated from the reaction mixture by centrifugation.

For attaching a biosensor peptide to aminated beads, the peptide is esterified at its C-terminus, during solid-phase synthesis, to contain a N-hydroxysuccinimide ester (NHS ester). NHS-esterified peptide is combined with aminated beads in an aqueous solution at neutral pH (e.g., 50 mM PBS, 150 mM NaCl, at a pH of 7.0-7.5 or 50 mM Tris, 150 mM NaCl, at a pH of 7.0-7.5) and incubated for two hours at ambient temperature, during which time the NHS-activated carboxyl groups of the peptide react spontaneously with amino groups on the bead. Coated beads are then separated from the reaction mixture by centrifugation.

Beads functionalized with streptavidin are also commercially available (e.g., Spherotech, Lake Forest, IL). These can be used for immobilization to the surface of wells coated with biotin (available from G-Biosciences, St. Louis, MO). In addition, beads functionalized with biotin are commercially available (e.g., Spherotech, Lake Forest, IL).

### Example 11: Determination of Biosensor Density

A statistical mechanical model of multivalent adhesion, based on the binding of influenza virus to a host cell, was adapted to the interaction between SARS-CoV-2 and its host cell; and used to design a solid support containing a plurality of biosensors (i.e., a cell decoy) that binds specifically to the SARS-CoV-2 spike protein. Xu & Shaw (2016) Biophys. J. 110:218-233. The multivalent adhesion model for binding between two particles provides a means to optimize the COVID-19 test for different conditions of infection (e.g., different viral load ranges; diagnostic or screening applications). This example provides a brief summary of these calculations to demonstrate how the density of biosensors (i.e., binding moiety+fluorescent moiety) on a solid surface can be adjusted for maximal sensitivity and specificity.

In a multivalent adhesion process the effective dissociation constant for adhesion (K_{D,AH}) is defined as:

RT In K_{d,AH}=RT In K₀+ΔG_{n̅}

K₀ is the 0-valency dissociation constant between the two surfaces when no specific interactions occur, and is obtained from:

$${K}_{0} = \frac{{N}_{R} {N}_{L}}{{M}_{R} {M}_{L}} {v}_{eff}^{- 1}$$

in which N_{R} is the number of binding moieties at the interaction interface, N_{L} is the number of spike protein receptor binding domains at the interaction interface, M_{R} is the total number of binding sites per solid support, M_{L} is the total number of binding sites per viral particle, and ν_{eff} is the effective molar volume in which binding between the binding moiety and the spike protein occurs.

ΔGn̅ is the average free energy of binding between the two particles, which is obtained from:

$$Δ {G}_{\overline{n}} = \overline{n} Δ G - RT ln [ ( \begin{matrix}{N}_{R} \\ \overline{n}\end{matrix} ) ( \begin{matrix}{N}_{L} \\ \overline{n}\end{matrix} ) \overline{n} ! ]$$

in which ΔG is the free energy of binding between a single binding moiety and a single spike protein S1 domain, and n is the average number of binding moiety-S1 domain interactions that occur at the interface.

In summary, the effective dissociation constant for adhesion can be computed from the dissociation constant for a single binding moiety-RBD interaction, the distribution of interacting sites on both the virus and the solid surface, and ν_{eff}, which is a parameter that depends on the geometry of the two interacting surfaces.

As an estimate for ν_{eff}, the value previously obtained for the adhesion of the influenza virus to host cells was used, since influenza and SARS-CoV-2 have similar size and shape, with effective volumes of approximately 2.7×10⁶ M⁻¹. Xu & Shaw, supra. For SARS-CoV-2 M_{L} is 222 (i.e., 74 spike protein trimers per viral particle). M_{R} is controllable by the coverage density of the solid surface (e.g., bead) with the biosensor (e.g., 1,000 sensor protein molecules). ΔG is obtained from the dissociation constant for the binding of biosensor to a single spike protein receptor binding domain as ΔG=RT lnK_{d}. Binding of the ACE2 receptor α-helix binding moiety (Example 6) to the spike protein with a K_{d} of 50 nM yields a ΔG of −41 kJ/mol. Estimating the interaction surface of the SARS-CoV-2 virus to be about one third of its total surface area (similar to that of influenza virus), yields a N_{L} of 24 possible spike proteins (8 trimers) to interact with a bead.

The average number of interactions between a viral particle and bead is controlled by adjusting the coverage density of biosensor on the solid surface (M_{R}). For instance, a 1 μm bead (surface area 3.14×106 nm²) comprising 1,000 biosensors has a density of one biosensor per 3,140 nm², or 8 biosensors on the virus reciprocal interaction area (N_{R}). In such a configuration, an average of ˜4 interactions between bead and viral particle is estimated. Using this estimate and the parameters provided above, the dissociation constant for adhesion of the virus to one biosensor-containing bead is about ˜10⁻⁴¹ M. In other words, the beads act as thermodynamic sinks for the virus, so any viral particle present in a clinical sample (even a single particle) will adhere to a bead, provided that there are enough beads to accommodate them all.

### Example 12: Assembly of Detection System

Sensor beads are attached to a surface in a number of ways. In certain embodiments, beads with dual functionalization (carboxyl groups and streptavidin) can be used. The biosensor is attached to the bead via the carboxyl groups (as described, for example, in Example 10 above) and the resultant sensor bead is attached to a biotinylated surface (e.g., a biotinylated microwell) by its bound streptavidin. Beads with streptavidin functionalization (sparse coating), that can be used in such methods for immobilization to the surface of wells coated with biotin, are commercially available (e.g., G-Biosciences, St. Louis, MO).

In additional embodiments, carboxylated beads are reacted with both streptavidin (which contains reactive amino groups at its N-terminus) and a biosensor, and are attached to a biotinylated surface (e.g., a biotinylated microwell). Avidin-biotin binding occurs in aqueous solution at ambient temperatures.

The density of sensor beads on the surface is chosen to provide maximal sensitivity, as described elsewhere herein.

In various aspects of the technology described herein, exemplary spectrally paired fluorophore donor and fluorophore acceptor, or spectrally paired fluorophore donor and dark quencher for use in the methods, compositions and kits may include, but not limited tol) protein-protein pairs, selected from the group consisting of ECFP-Citrine, ECFP-Venus, Cerulean-Citrine, Cerulean-Venus, Cerulean-Ypet, Cerulean-YFP, CyPet-EYFP, CyPet-Venus, CyPet-YPet, CyPet-Citrine, mTurquoise-Venus, mTurquoise-Ypet, mTurquoise-Citrine, ECFP-EYFP, TagGFP-TagRFP, mTFP1-Citrine, Citrine-mKate2, mTurquoise1-SEYFP, mTurquoise2-SEYFP and clover-mRuby2, 2) protein-organic dye pairs, selected from the group consisting of EGFP-mCherry, SYFP2-mStrawberry, mTFP1-mOrange, Clover-mCherry, GFP-Cy3, YFP-Cy3, ECFP-BHQ-0, EYFP-BHQ-2, EGFP-Cy3 and EGFP-BHQ-1, 3) organic dye-organic-dye pairs, selected from the group consisting of mOrange-mCherry, Alexa488-Alexa555, Alexa488-Cy3, Alexa 568-Alexa633, Cy3-Cy5, Alexa 488-Alexa514, Alexa488-Alexa532, Alexa488-546, Alexa488-610, Alexa647-Alexa 680, Alexa647-Alexa680, Alexa647-Aelxa700, Alexa647-Alexa750, BHQ-1-FAM, BHQ-1-TET, BHQ-1-JOE, BHQ-1-HEX, BHQ-1-Oregon green, BHQ-2-TAMRA, BHQ-2-ROX, BHQ-2-Cy3, BHQ-2-Cy3.5, BHQ-2-CAL Red, BHQ-2-Red 640, BHQ-3-Cy5, or BHQ-3-Cy5.5, Dabcyl-Edans and Dabsyl-Edans, fluorescine.

### Embodiments

Although the above description and the attached claims disclose a number of embodiments of the present invention, other alternative aspects of the invention are disclosed in the following further embodiments.

Embodiment 1: A composition for detection of a target, comprising: (a) a binding moiety that binds to a molecule on the surface of the target; and (b) a fluorescent moiety having: (i) a first emission property when the binding moiety is not bound to the target, and (ii) a second emission property when the binding moiety is bound to the target, wherein the first and second emission properties are selected from the group consisting of one or both of fluorescence intensity and emission wavelength.

Embodiment 2. The composition of claim 1, wherein the binding moiety is a peptide or a protein.

Embodiment 3. The composition of claim 1 wherein the second emission property is: (i) increased fluorescence intensity, and (ii) higher emission wavelength.

Embodiment 4. The composition of claim 3 wherein the fluorescent moiety is 2-(2-furyl)-3-hydroxychromone (FHC).

Embodiment 5. The composition of claim 1 wherein the second emission property is: (i) increased fluorescence intensity, and (ii) lower emission wavelength.

Embodiment 6. The composition of claim 5 wherein the fluorescent moiety is 6-N,N-dimethylamino-2,3-naphthalimide (6-DMN).

Embodiment 7. The composition of claim 1 wherein: (a) the first emission property is a ratio of fluorescence intensity at a first wavelength and fluorescence intensity at a second wavelength; and (b) the second emission property is a change in the ratio.

Embodiment 8. The composition of claim 7, wherein the fluorescent moiety is a combination of Alexa488 and Alexa594.

Embodiment 9. The composition of claim 1 wherein the second emission property is decreased fluorescence intensity.

Embodiment 10. The composition of claim 9, wherein the fluorescent moiety is Atto 655.

Embodiment 11. The composition of claim 1, wherein the target is a cell.

Embodiment 12. The composition of claim 1, wherein the target is a virus.

Embodiment 13. The composition of claim 12, wherein the virus is SARS-CoV-2.

Embodiment 14. The composition of claim 13, wherein the binding moiety binds to the SARS-CoV-2 spike (S) protein.

Embodiment 15. The composition of claim 14, wherein the binding moiety is a peptide comprising a portion of the peptidase domain of angiotensin converting enzyme-2 (ACE2).

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. The term “about” unless otherwise indicated is used in the context of describing ±5% or less for a defined value. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A composition, comprising:
an engineered protein including at least one amino acid sequence that binds to at least one target molecule;
a donor moiety and an acceptor moiety fused to the engineered protein;
wherein the engineered protein undergoes a first conformation change to unfold in the absence of the target molecule and undergoes a second conformational change to fold upon binding to the target molecule using a fraction of a binding free energy provided by binding of the engineered protein to the target molecule or wherein the engineered protein undergoes the second conformation change to fold in the absence of the target molecule and undergoes the first conformation change to unfold upon binding to the target molecule; and
wherein responsive to the first or the second conformational change, an energy transfer or an electron transfer occurs between the donor moiety and the acceptor moiety.

2. The composition of claim 1, wherein the engineered protein comprises two domains connected by a structural spacer.

3. The composition of claim 2, wherein the structural spacer is a helical linker.

4. The composition of claim 1, wherein the donor moiety is a fluorescence donor moiety and the acceptor moiety is a fluorescence acceptor moiety, and wherein responsive to the first or the second conformational change, Förster resonance energy transfer occurs between the fluorescent donor moiety and the fluorescence acceptor moiety.

5. The composition of claim 1, wherein the donor moiety is a quencher and the acceptor moiety is a fluorescence acceptor moiety, and wherein responsive to the first or the second conformational change, photo-induced electron transfer occurs between the quencher and the fluorescence acceptor moiety.

6. The composition of claim 5, wherein the quencher is tryptophan and the fluorescence acceptor is ATTO655.

7. The composition of claim 1, wherein the engineered protein is encapsulated into synthesized biotinylated liposomes.

8. The composition of claim 7, wherein the biotinylated liposomes are tethered to a surface via biotin-streptavidin-biotin linkages.

9. The composition of claim 1, wherein the engineered protein spontaneously forms nanoscale assemblies of a selected number of copies and symmetry in configurations that enable multi-binding to the target molecule.

10. The composition of claim 1, wherein the engineered protein is fused to an oligomerization domain through a flexible linker.

11. The composition of claim 10, wherein the oligomerization domain is a BMC domain of a bacterial beta carboxysome assembly.

12. The composition of claim 1, wherein a number of copies of the engineered protein are covalently linked to one or more substrates at a calculated density per substrate.

13. The composition of claim 12, wherein a number of copies of the nanoscale assemblies are coated on to one or more substrates at a calculated density per substrate.

14. A composition for detection of a target, comprising:
a downhill folding protein including a binding moiety that binds to a molecule on the surface of the target; and
a fluorescent moiety fused to the downhill folding protein, the fluorescent moiety having a first emission property when the binding moiety is not bound to the target, and a second emission property when the binding moiety is bound to the target,
wherein the first and second emission properties are selected from the group consisting of one or both of fluorescence intensity and emission wavelength; and
wherein the downhill folding protein undergoes a conformational change responsive to binding to the target

15. The composition of claim 14, wherein the downhill folding protein or an oligomer of the downhill folding protein is coated onto a plurality of substrates at a desired density per substrate to generate hierarchical assemblies of the downhill folding protein or the oligomer, each hierarchical assembly having a shape and/or a size that resemble a host cell of the target to enhance affinity and specificity via engineered multivalent adhesion.

16. The composition of claim 14, wherein the binding moiety is a peptide or a protein.

17. The composition of claim 14, wherein the second emission property is increased fluorescence intensity, and higher emission wavelength.

18. The composition of claim 14, wherein the second emission property is increased fluorescence intensity, and lower emission wavelength.

19. The composition of claim 14, wherein the first emission property is a ratio of fluorescence intensity at a first wavelength and fluorescence intensity at a second wavelength; and the second emission property is a change in the ratio.

20. The composition of claim 14, wherein the second emission property is decreased fluorescence intensity.

21. The composition of claim 14, wherein the fluorescent moiety is Atto 655.

22. The composition of claim 14, wherein the target is a virus.

23. The composition of claim 22, wherein the virus is SARS-CoV-2.

24. A composition for detecting a target, the composition comprising:
a plurality of responsive proteins;
wherein the plurality of responsive proteins are assembled on to a plurality of substrates through engineered multivalent adhesion at a desired density per substrate to generate a host cell decoy for the target.

25. The composition of claim 24, wherein the responsive protein is a downhill folding protein; wherein the responsive protein undergoes a conformational change responsive to binding the target, and wherein the responsive protein is fused to a donor moiety and an acceptor moiety.

26. The composition of claim 24, wherein each assembly of the plurality of substrates coated with the plurality of responsive proteins have a shape and/or a size that resemble a host cell of the target to enhance affinity and specificity.

27. The composition of claim 24, wherein the plurality of responsive proteins include oligomers of the responsive proteins.

28. The composition of claim 25, wherein the responsive protein undergoes a first conformation change to unfold in the absence of the target and undergoes a second conformational change to fold upon binding to the target using a fraction of a binding free energy provided by binding of the responsive protein to the target molecule or wherein the responsive protein undergoes the second conformation change to fold in the absence of the target and undergoes the first conformation change to unfold upon binding to the target.

29. The composition of claim 24, wherein the target is a virus.


SEQUENCE LISTING (23 sequences)

SEQ ID NO: 1 - protein, 6 aa, Artificial Sequence
  tetracysteine motif
  misc_feature (3)..(4): Xaa can be any naturally occurring amino acid
CCXXCC

SEQ ID NO: 2 - protein, 5 aa, Artificial Sequence
  cysteine motif
  misc_feature (2)..(4): Xaa can be any naturally occurring amino acid
CXXXC

SEQ ID NO: 3 - DNA, 6 bp, Homo sapiens
taatta

SEQ ID NO: 4 - protein, 119 aa, Artificial Sequence
  bacterial
MSIAVGMIETRGFPAVVEAADSMVAAARVTLVGYEKIGSGRVTVIVRGDVMGVQASVSAGIEAANRVNGGEVLSTHIIAAPHENLEYVLPIRYTEEVEQFRTYGVPRGGSHMLKEVWEE

SEQ ID NO: 5 - protein, 35 aa, Homo sapiens
STIEEQAKTFLDKFNHEAEDLFYQSSLASWNYNTN

SEQ ID NO: 6 - protein, 24 aa, Homo sapiens
KAVCHPTAWDLGKGDFRILMCTKV

SEQ ID NO: 7 - protein, 40 aa, Homo sapiens
  MOD_RES (21)..(21): Cysteine fluorophore
GSGSGSTIEEQAKTFLDKANCEAEDLAYQSSLASWNYNTN

SEQ ID NO: 8 - protein, 55 aa, Homo sapiens
SSGSGSTIEEQAKTFLDKANHEAEDLCYQSSLASYNYNTNLPETGGGKAVCHPTA

SEQ ID NO: 9 - protein, 16 aa, Homo sapiens
WDLGKGDQRTLMCTKV

SEQ ID NO: 10 - protein, 55 aa, Homo sapiens
SSGSGSTIEEQAKTFLDKFNHECEDLAYQSSLASYNYNTNLPETGGGKAVCHPTA

SEQ ID NO: 11 - protein, 16 aa, Artificial Sequence
  Synthetic peptide
  MOD_RES (8)..(8): Para-acetlyphenylalanine
WDLGKGDFRTLMCTKV

SEQ ID NO: 12 - protein, 34 aa, Homo sapiens
  MOD_RES (21)..(21): Cysteine fluorophore
GSGSGSTIEEQAKTFLDKANCEAEDLAYQSSLAS

SEQ ID NO: 13 - protein, 55 aa, Homo sapiens
  MOD_RES (23)..(23): Para-acetlyphenylalanine
SSGSGSTIEEQAKTFLDKFNHEFEDLAYQSSLASYNYNTNLPETGGGKAVCHPTA

SEQ ID NO: 14 - protein, 17 aa, Homo sapiens
WDLGKGDFCRTLMCTKV

SEQ ID NO: 15 - protein, 38 aa, Homo sapiens
STIEEQAKTFLDKFNHEAEDLFYQSSLASWNYNTNITE

SEQ ID NO: 16 - protein, 45 aa, Homo sapiens
SSGSGSTIEEQAKTFLDKANHEAEDLCYQSSLASYNYNTNLPETG

SEQ ID NO: 17 - protein, 26 aa, Homo sapiens
GGKAVCHPTAWDLGKGDQRTLMCTKV

SEQ ID NO: 18 - protein, 71 aa, Homo sapiens
SSGSGSTIEEQAKTFLDKANHEAEDLCYQSSLASYNYNTNLPETGGGKAVCHPTAWDLGKGDQRTLMCTKV

SEQ ID NO: 19 - protein, 45 aa, Homo sapiens
  MOD_RES (23)..(23): Para-acetlyphenylalanine
SSGSGSTIEEQAKTFLDKFNHEFEDLAYQSSLASYNYNTNLPETG

SEQ ID NO: 20 - protein, 27 aa, Homo sapiens
GGKAVCHPTAWDLGKGDFCRTLMCTKV

SEQ ID NO: 21 - protein, 72 aa, Homo sapiens
  MOD_RES (23)..(23): Para-acetlyphenylalanine
SSGSGSTIEEQAKTFLDKFNHEFEDLAYQSSLASYNYNTNLPETGGGKAVCHPTAWDLGKGDFCRTLMCTKV

SEQ ID NO: 22 - protein, 775 aa, Homo sapiens
KFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 23 - DNA, 108 bp, Artificial Sequence
  Plasmid
tataccatggattacaaggatgacgacgataagggatccgagatctggaattcggtaccgagctcaagcttgcggccgcctatagtgtcacctaaatcgtaacccagc